SEQUENCE LISTING (24 sequences)

SEQ ID NO: 1 - DNA, 1317 bp, Mus musculus
  CDS (1)..(1317)
atgtcgagacgtgacgtggtccttaccaatgttactgttgtccagctacggcgggaccgatgcccatgcccatgcccatgtccatgcccatgccctgtgatcagaccacctccacccaaggttgaggatccaccacccacggttgaagaacagccaccgccaccgccgccgccacctccacctccaccaccacctcctcctcctcctccaccccagatagagccagacaagtttgaagaggctccccctccccctccccctcctcctcctcctccccctccccctcccccaccactccaaaagccagctagagagctgacagtgggtatcaatggatttggacgcattggtcgtctggtgctgcgagtctgcatggagaagggcattagggtggtagcagtgaatgacccattcattgatccagaatacatggtttacatgttcaaatatgactccacacatggtagatacaaaggaaacgtggaacataagaatggacaactagttgtggacaaccttgagatcaacacgtaccagtgcaaagaccctaaagaaatcccctggagctctatagggaatccctacgtggtggagtgtacaggcgtctatctgtccatcgaggcagcttcggcacatatttcatctggtgccaggcgtgtggtggtcactgcaccctcccccgatgcacccatgtttgtcatgggagtgaacgagaaggactataaccctggctctatgaccattgtcagcaatgcatcctgtaccaccaactgcctggctcctctcgccaaggttattcatgaaaacttcgggatcgtggaagggctaatgaccacagtccattcctacacagccactcagaagacagtggatgggccatcaaagaaggactggcgaggtggccgcggcgctcaccaaaacatcatcccatcgtccactggggctgccaaggctgtaggcaaagtcatcccagagctcaaagggaagctaacaggaatggcattccgggtgccaaccccaaacgtgtcagttgtggacctgacctgccgcctggccaagcctgcttcttactcggctatcacggaggctgtgaaagctgcagccaagggacctttggctggcatccttgcttacacagaggaccaggtggtctccacggactttaacggcaatccccattcttccatctttgatgctaaggctggaattgccctcaatgacaacttcgtgaagcttgttgcctggtacgacaacgaatatggctacagtaaccgagtggtcgacctcctccgctacatgtttagccgagagaagtaa

SEQ ID NO: 2 - protein, 438 aa, Mus musculus
MSRRDVVLTNVTVVQLRRDRCPCPCPCPCPCPVIRPPPPKVEDPPPTVEEQPPPPPPPPPPPPPPPPPPPPQIEPDKFEEAPPPPPPPPPPPPPPPPPLQKPARELTVGINGFGRIGRLVLRVCMEKGIRVVAVNDPFIDPEYMVYMFKYDSTHGRYKGNVEHKNGQLVVDNLEINTYQCKDPKEIPWSSIGNPYVVECTGVYLSIEAASAHISSGARRVVVTAPSPDAPMFVMGVNEKDYNPGSMTIVSNASCTTNCLAPLAKVIHENFGIVEGLMTTVHSYTATQKTVDGPSKKDWRGGRGAHQNIIPSSTGAAKAVGKVIPELKGKLTGMAFRVPTPNVSVVDLTCRLAKPASYSAITEAVKAAAKGPLAGILAYTEDQVVSTDFNGNPHSSIFDAKAGIALNDNFVKLVAWYDNEYGYSNRVVDLLRYMFSREK

SEQ ID NO: 3 - DNA, 1450 bp, Rattus norvegicus
  CDS (4)..(1302)
gccatgtcgagacgtgacgtggttcttaccaatgttactgttgtccagctacggcgggacccatgcccatgtccatgcccatgtccatgtccatgtccatgtcccgtgatcagaccacctccacctccacccaaggttgaggagcctccgccacccaaggaggagccgccgccaccgccaccaccgcctcctccaccccagatagagccagaggagcctaaagaggctccacctccacctccacctcctccaccccctccccctccccctcctccaccacccccaaagccagctaaagagctaacagtgggcatcaatggatttggacgcattggtcgtctggtgctacgagtctgcatggagaagggcgttcgggtagtagcagtgaatgacccattcattgatccagaatacatggtgtacatgttcaaatatgactccacacatggtagatacaaaggaacggtggaacataagaatggacgactagttgtggacaaccttgagatcaacgtgttccagtgcaaagaacctaaggaaatcccctggagctctgtagggaatccctacgtggtggaagccacaggtgtatacctgtccatagaggcggcttcggggcacatttcatctggtgccaggcgtgtcatcgtcactgcaccttcccccgatgcacccatgttggtcatgggtgtgaatgagaaggactataaccctggctccatgaccgttgtcagcaatgcatcctgtaccaccaactgcctggcccccctcgctaaggttattcacgaacgcttcgggatcgtggaagggctaatgaccaccgtccatgcctacacggccactcagaagacagtggacgggccatcaaagaaggactggcgaggtggccgaggtgcccaccagaacatcatcccatcttccactggggctgccaaggctgtaggcaaagtcatcccagagctcaatgggaagttaacaggaatggcattccgggtgccaaccccaaacgtatcagttgtggacctgacctgccgcctggctcagcctgcttcctacactgctatcaaggaggctgtgaaagcagcagccaagggacctatggctggcatccttgcttatacagaggaccaggtggtctccacggactttaatggcgattcccattcttccatctttgatgctaaggctggaattgccctcaatgacaacttcgtgaagcttgtttcctggtacgacaacgaatatggctacagtcaccgggtagtcgacctcctccgctacatgtttagccgagagaaataaaggcccctccttgctcccctgcgcacctcgcgtacctgccttcggctccccttcaaaagagggcgcacttcgcgctgccgccgggtcaacaatgaaataaaaaccagagtgcgcacagaaaaaaaaaaaaaaaaaaaaaaaaaaaaaa

SEQ ID NO: 4 - protein, 432 aa, Rattus norvegicus
MSRRDVVLTNVTVVQLRRDPCPCPCPCPCPCPCPVIRPPPPPPKVEEPPPPKEEPPPPPPPPPPPQIEPEEPKEAPPPPPPPPPPPPPPPPPPPKPAKELTVGINGFGRIGRLVLRVCMEKGVRVVAVNDPFIDPEYMVYMFKYDSTHGRYKGTVEHKNGRLVVDNLEINVFQCKEPKEIPWSSVGNPYVVEATGVYLSIEAASGHISSGARRVIVTAPSPDAPMLVMGVNEKDYNPGSMTVVSNASCTTNCLAPLAKVIHERFGIVEGLMTTVHAYTATQKTVDGPSKKDWRGGRGAHQNIIPSSTGAAKAVGKVIPELNGKLTGMAFRVPTPNVSVVDLTCRLAQPASYTAIKEAVKAAAKGPMAGILAYTEDQVVSTDFNGDSHSSIFDAKAGIALNDNFVKLVSWYDNEYGYSHRVVDLLRYMFSREK

SEQ ID NO: 5 - DNA, 1455 bp, Homo sapiens
  CDS (67)..(1293)
ggcggtccgcacgcacctcggtaacatcacagcaggtccaggccaatgataaccttataagaggccatgtcgaagcgcgacatcgtcctcaccaatgtcaccgttgtccagttgctgcgacagccgtgcccggtgaccagagcaccgcccccacctgagcctaaggctgaagtagagccccagccacaaccagagcccacaccagtcagggaggaaataaagccaccaccgccaccactgcctcctcaccccgctactcctcctcctaagatggtgtctgtggcccgggagctgactgtgggcatcaatggatttggacgcatcggtcgcctggtcctgcgcgcctgcatggagaagggtgttaaggtggtggctgtgaatgatccattcattgacccggaatacatggtgtacatgtttaagtatgactccacccacggccgatacaagggaagtgtggaattcaggaatggacaactggtcgtggacaaccatgagatctctgtctaccagtgcaaagagcccaaacagatcccctggagggctgtcgggagcccctacgtggtggagtccacaggcgtgtacctctccatacaggcagcttcggaccacatctctgcaggtgctcaacgtgtggtcatctccgcgccctcaccggatgcaccaatgttcgtcatgggtgtcaatgaaaatgactataaccctggctccatgaacattgtgagcaacgcgtcctgcaccaccaactgtttggctcccctcgccaaagtcatccacgagcgatttgggatcgtggaagggttgatgaccacagtccattcctacacggccacccagaagacagtggacgggccatcaaggaaggcctggcgagatgggcggggtgcccaccagaacatcatcccagcctccactggggctgcgaaagctgtgaccaaagtcatcccagagctcaaagggaagctgacagggatggcgttccgggtaccaaccccggatgtgtctgtcgtggacctgacctgccgcctcgcccagcctgccccctactcagccatcaaggaggctgtaaaagcagcagccaaggggcccatggctggcatccttgcctacaccgaggatgaggtcgtctctacggacttcctcggtgatacccactcgtccatcttcgatgctaaggccggcattgcgctcaatgacaatttcgtgaagctcatttcatggtacgacaacgaatatggctacagtcaccgggtggtcgacctcctccgctacatgttcagccgagacaagtgaaacgggaaggtcctttctttccttcccaggggccggggccggaacatgtgcctcccgttccagcatctggctgcccgggggaggaaggacacccggggcgggcgccccacgccgatgggtccatggtgaaataaaaaacagtgctcgaaaaaaaaaaaaaaa

SEQ ID NO: 6 - protein, 408 aa, Homo sapiens
MSKRDIVLTNVTVVQLLRQPCPVTRAPPPPEPKAEVEPQPQPEPTPVREEIKPPPPPLPPHPATPPPKMVSVARELTVGINGFGRIGRLVLRACMEKGVKVVAVNDPFIDPEYMVYMFKYDSTHGRYKGSVEFRNGQLVVDNHEISVYQCKEPKQIPWRAVGSPYVVESTGVYLSIQAASDHISAGAQRVVISAPSPDAPMFVMGVNENDYNPGSMNIVSNASCTTNCLAPLAKVIHERFGIVEGLMTTVHSYTATQKTVDGPSRKAWRDGRGAHQNIIPASTGAAKAVTKVIPELKGKLTGMAFRVPTPDVSVVDLTCRLAQPAPYSAIKEAVKAAAKGPMAGILAYTEDEVVSTDFLGDTHSSIFDAKAGIALNDNFVKLISWYDNEYGYSHRVVDLLRYMFSRDK

SEQ ID NO: 7 - DNA, 1303 bp, Homo sapiens
  CDS (64)..(1068)
gttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccaaaaaaaaaaaaaaaaaaaaaaaaaaaaaaaaaaa

SEQ ID NO: 8 - protein, 334 aa, Homo sapiens
GKVKVGVNGFGRIGRLVTRAAFNSGKVDIVAINDPFIDLNYMVYMFQYDSTHGKFHGTVKAENGKLVINGNPITIFQERDPSKIKWGDAGAEYVVESTGVFTTMEKAGAHLQGGAKRVIISAPSADAPMFVMGVNHEKYDNSLKIISNASCTTNCLAPLAKVIHDNFGIVEGLMTTVHAITATQKTVDGPSGKLWRDGRGALQNIIPASTGAAKAVGKVIPELNGKLTGMAFRVPTANVSVVDLTCRLEKPAKYDDIKKVVKQASEGPLKGILGYTEHQVVSSDFNSDTHSSTFDAGAGIALNDHFVKLISWYDNEFGYSNRVVDLMAHMASKE

SEQ ID NO: 9 - protein, 9 aa, Homo sapiens
NASCTTNCL

SEQ ID NO: 10 - protein, 13 aa, Mus musculus
PSKKDWRGGRGAH

SEQ ID NO: 11 - protein, 13 aa, Homo sapiens
PSRKAWRDGRGAH

SEQ ID NO: 12 - protein, 12 aa, Homo sapiens
PSGKLWRGGRGA

SEQ ID NO: 13 - protein, 6 aa, Mus musculus
YLSIEA

SEQ ID NO: 14 - protein, 6 aa, Homo sapiens
YLSIQA

SEQ ID NO: 15 - protein, 6 aa, Homo sapiens
FTTMEK

SEQ ID NO: 16 - protein, 5 aa, Homo sapiens
PSPDA

SEQ ID NO: 17 - protein, 5 aa, Homo sapiens
PSADA

SEQ ID NO: 18 - protein, 8 aa, Homo sapiens
MTTVHSYT

SEQ ID NO: 19 - protein, 8 aa, Homo sapiens
MTTVHAIT

SEQ ID NO: 20 - protein, 19 aa, Mus musculus
MSRRDVVLTNVTVVQLRRD

SEQ ID NO: 21 - protein, 19 aa, Homo sapiens
MSKRDIVLTNVTVVQLLRQ

SEQ ID NO: 22 - DNA, 11462 bp, Mus musculus
aaattttatgtctgagtgcttgcctgtatgtatgtgtggaacctgtgcgtacgggtgcctgcagaggccagaagagggcatcagatcctctggaactggagttacacaatgttgtgaacttctacgtgggtgctgggatctgaactcgagtcctctgtgagagcaacaaatgctcttaactactgagtgactttcctgtccctcactctgaggatttaatctctggattcttagtgcttggaggcttgggggtgcgagtgtgtaacagcacacatacataggccataatgaccagtcaggagcttctgctgtctgtaaaggtgcagggacctgaactcaggttgtcaactttgcaccgcaagggggttttgcaggtgagttaactggagcctgcaccagttcacaaatcttgccttcaaccacaggcattaagtgagctacctaaggctgtcactgagagggtagatctcaggggccatgcagcatgagacactggatacattctaaaagtatgtgggatttttgaggatgttgaagattaaattcagagctctcctgcctgctgggcaagccctctaccattgagctgtgtttctagtcccttaaagttttagaataaacgttcaaaaaattactttatcttatataaaaggcaataacatcaccattgaaattctctacacaaaaaattttaattcattttaccacatattctatgatatttttatttatggaatcagtttttttcttttaatttttaactttattagtgtttgtgtgtgtgcctgcaaactccagtcaggtcagtgtgtaggtctgggccactcatgagtcatcctgtctcttcttctacaaagtgaggcaggctctcttgtttctgcagcattgggggagggagcatccctttgctttaacggtatcaactatgctccaatggatggcccaggccccttatgcatagcccaaattgagtcagtggagtagcaaaaggaaacaagcctagctgacccatgagagtctggccttagagaaccaagccatcacacctggcttttaaatatatcctatgtttactgaaagcatgagagactccatacttgatcattttcaacactctctcctccctgccaactacttgggagtttgccttgtttccttttgctactccactgactcaatttgggccatgcataaggggcctgggccatccattggagcatagttgataccgttaaagcaaagggatgctccctcccccagaagtcaccagctatccatagctccccaggcagaggtgggtgcgtgtaagcctctaccctccatagtggaaggctgagcggcatgaccttgtgcagttctgatgcaggcagcgacagctgctgagtctcctcccacatcgcaggcttttatagaccatatttctgcctccccttctgccatgataaggaccacttcagttacagacagcacttgagaggatcccaagccatcaaggggagacctgggagaggctttctcagcatttccacacacaggggagagcaagggaaatgcctcagagatccaggaacagcatagagcaggtggaccatggttgggaggaagagagatggcagccatgagcttctgtgtgaacctagtgggttccactctagatgccagagggtggccttgaaatcctcagtgtgggactctcaaaagcttctggagagtgggctgaggtctttgccctgcacaccactctctatgtgatgtcattgatcaatgtgacatcatcagaagctcaagagccactgggaccttgggagcttcacacctcagtaacaccacggaggggggccaaggcagccaggccatgagatcttaggccatgtcgagacgtgacgtggtccttaccaatgttactgttgtccagctacggcgggaccgatgcccatgcccatgcccatgtccatgcccatgccctggtaaggagagttgttgggggtggccctgttaagaggaggaaaatgtggtctgggagtctctccaccctcccccagggtacttagagtcctgagaccctcccccaccagcatgctctctactgggtgctcctgcaggcacaggccagggtgggcccttcaggctctctcctctgggatccttcctcgccccagagagcttctgcttgggatggggttggggttgggagctctgccctcaggactgcctgacctttgggttctatcagtggagaggggagaagcagaggccaggaggaggcagggaggctggagagggagaggaaaccaggagcatttggaacaggtctaggtgggtgtgccccccacatttcattgatgtccctgctggccagggacaagggggcttggtaggcaggtcctccctgggctgtagggagcaagggtacttgcagccctccatggcaaggctgctccctttacaggcaagggaggcatgtgttcactgagtctcacagccaccaggtgaggcctgggtgtttcactgtccccattctgtggaggccagcaaagatcatgagaggccaaaccacttcccaggctcccaaccaaggaagaaatggtggagctgggactctcctagggcctcagtctctgtacaacctgctgccagctgctgggcaaacccagccagaggctgtttcaggtgctgaaggtaaggaaaggaagtgggtaagagtcaaggagggattgatgtcacaacatgtgtggaatgtttcatctgtatttcacagtataagaaactagcacaaaaaatagaccctcatgttaccgctaaacacacagacatacagacacacacgcatacacgcacacatgcatgctgaaacagaagttcacataccttaccctcagggctcttgcttgttctctggctcagagggtttggaaaagaaccaacccatagttggcccctagggctcaaagcccactggtttctgtatggtaaagactgctttttcacactttgtgtgcgtgtgaagataaaggtcacaggacaatttatggaagttgtttttgtttctaccatgtggtgtccaggattgacactcaggtcacacgcctctgggacaagcacccactgaggtgtctcaccatccccacgtcttatgtgagggacccagttcaccacctcccttctgaactctctgctctctctctacagtgatcagaccacctccacccaaggttgaggatccaccacccacggttgaagaacagccaccgccaccgccgccgccacctccacctccaccaccacctcctcctcctcctccaccccagatagagccagacaagtttgaagaggctccccctccccctccccctcctcctcctcctccccctccccctcccccaccactccaaaagccagctagagagctgacagtgggtatcaatgggtgagtatgtcagaccccaggtgatgctggagccagccagccccaaaggctgacactgtggcctccttggatcactatgggctggggacatcttccttccttctcactgggcaggccagtcctcagagaactcccttctaggagacaacgatcagatagagaattggaagtagttcaagtgcaggctgggccacagaaggcctctggaaagaggcctgaaagacaggctgacaaggcagccagctgtctctctacaggggcaaatttttgaggtagtgggacaagtaatgactctgacttgaaagagacatgcttactacattcaaagacagccaggaaatcttcacagctgtaagaacaaattaggggtaagggaagggggagagggagtcctttaagacctgaagcaccatggaaaggaatatagatcttgtttacctgagagagggaggtggttttcacatggagacatacggcccttccctgggaacacccagctataggagtgagcatgagggacattattaggaaggcatggcagtgtgcctgtcacactagcagcaagggaggctaaaacaggataaaactgaggatgccaggcttcacagcaagcgcctttacctactgagctgtcttaccagctccaagtgttcaccccttaataacccccaatggttcaaagttcaacatgaactttggtagggcactgacaccatgacatctctgtcactctggttctcacacccattccctttgctttgtgttcctgattggacagatttggacgcattggtcgtctggtgctgcgagtctgcatggagaagggcattagggtggtagcagtgaatgacccattcattgatccagaatacatggtgagcagatggagggggcggggacagacggacagaccagcaaaagtggcacttgtgttggggagacgcttccaagagctatctagaagtctcctggctgcaatgcagaacctcctaaacctcttgcccacagtgggaactgtctaggtctgctcactctgcgacctactaccaggaccccactctgtctactgcaggtttacatgttcaaatatgactccacacatggtagatacaaaggaaacgtggaacataagaatggacaactagttgtggacaaccttgagatcaacacgtaccagtggtaaggacagaatgtgacaccatggctctaccatatttgcatgcctctaggtactgtttagagagagggtctcagaaaggtctctggcctctaaaacacttaaggacactcatctttaagactgccatggttcttacatgttttgtatttaacacatgtccgcatgtgttctctgcggcatgtgggttcactgtgccacatcctctgctttctctgagtcaccagccacctctctatggcaactgagccagttgctacccaggagcctctgaggccttttgattctccactgagccaaggttctcttctaccctcccccctcccaatgctcctgccccagctctcagggtccccggtgtgataagcatcccaccctgcattctctgtctcatctgccacacctggttatcacctctgggggcgctgggctgcacctcttacaccccagcaaggctgacttcctcactaggaagttctgttgatggtaagccccactgttaacagactcagaatctgtgtatttggcagtaacatgctaggtgtggggtgtacatgtgtaattcctgcacttgggaagccacaggaggattacctcaagtcctaggtcagcctgcagctacacagaaacaaaacagcataaaccagtaacaccttaccgtcactttgcaaacagagaagcacccaaccctctgtagccccaggcaaggtcttctggctccttctcaggcaagacactctcagcttcccttttaacttgctgaagatgaggtgtgacagagtttggaggaaggctgaaggcaggactggggaatcagggagaatagaggctgtttgccttgagtctgaaatcgtagccccaaaagcaattccagtcacccctatccttctgcccctgcctgggtgtcacccttcttcgcctccacacagcaccccattccacactgctagagaccagtggcagccatactttcaattctgaatgtttataaaatatatgtgtcaagccaagtaaaaagtaaacagcaagggatgaagagatagataggtagatcgttcacaggtagatcacaaaggatggagagatcgataggtaatagataggtaatagatagatcacaggtaggtaaggaagacagatgggtgggtggtgggtagatggttgaatggatagatggaaggatggaaggtgatggatggatgaatagatgggtgggtgggtgggtgggtgggtgggtgggtgggtggatggatggatggatggatggatggatggatggatagatagatagatagatagatagatagatagatagatagatagatagatagatggggatactataataagagttagttcccaggcagagctggtaaggcttccaagtaaaagaacaaatagcaacagaccctgatgagagaggcatcattgcagtcaggggctcacgatatccagcagacactggctatagaagccaaggtttcagttggtattcctgactgagctttctctcatggctgagcttacaagctctcccactggcaacactgcataccacaggataaacagtagtaacctacactcttggggactctgtgtttacctatgataagccatccctcctctgtcacagaggtgggagaggccagtccacctgaggacaaggggtcttgaaaaccacttgacataaacctgcttgggtttgaagggaggccagcctgacttccagagtcagcttctcagcaagccccaacaagcacataacaacttgccaacatcatacacaacacagttgccttccccatcagtgcaaccgaatgaataaatcttcacaacaaacctgtaggtcggaggggtcatgccaaactcaagttctattgcaggaattgccgaagtactctcaacctgaaagacagaaagtaggtgcatcagataaaagctccttgggccagatgaccgatgtgggctgactgagcagagggagaaaacggtcttgtggctagaagggtgggaaagcctctgcattcattcattcattcccacactattcctaatggaggacagaacccaggcctaggttcgcacactgccttcaccactgcctgacactggcttctctgaagcatgaacacagtagtaagtatgctggtgttccggtggttatcagttttctacagccaaaaaacctcagacagaaggcagcgcagggcacatgtgtagaatgactaaaagaaagacactgagactaggagtgaagttctgtagtagaataccttccactgcaggtgaggccctggcatcagtcctcagcatcacaaaaaaaattaaataaatactcattaattggggttgaagagacagcttatcagttatgaccacaaatggctttcagaggacccccaagttcaatttctagcattcacaggtggcatacctatggtatgaaactagcctgaagtagagccaagcaatcctaggaacaaatcttccagtgacatgagggacaagcccagacggcagtcacagagactgcttgctggacgcaggtagaaaacaaggataggtatctgacaagtttccagcacaaatatttgtcagtacatcaaggcaaagctggtcgtgatggtgtttgtgtagtcccagtgctaaggaggctgaggcagggagattgtggagattgcagaccagtaggggtcaaacaggagacacttatcaccagccaacaaacaaaaatcaaatgaaaacataggctgagacaggaacagaaagcctttctctggcttttctactgcccatgattggtccctgatattcacaggtgcttcagctgtaagcctcccctgagttgtcacctgatgtgtctgtctttggggacttcccaacaagggttcagtggtgataagagcctaaaagggctcaggtgagatggtcagagctgggggccaaagggcagcattggcagcctcaccatcttaggaatccaagctctgagcatgtgctgtaaatggttctggttctagtcagacacttgtgggcatggttcctgagagcctcagttgaaggtgtgaaagtctgtttagacacagcagggtatagcacacaagaaagggagtgtggctgacaggggcttgcaggacggcaccaacctggatcaggtaggtatatggagtttagggatcagatattagcagatcacataccaagacctctggctagcctgagatccaagcaagggaaactgacatagccttcccaccactccacagcaaagaccctaaagaaatcccctggagctctatagggaatccctacgtggtggagtgtacaggcgtctatctgtccatcgaggcagcttcggtaagtaggggagagtgcccagggctgcctgggaagggggcagtggtgcttggagaccctaaccttgtgctctatcactaggcacatatttcatctggtgccaggcgtgtggtggtcactgcaccctcccccgatgcacccatgtttgtcatgggagtgaacgagaaggactataaccctggctctatgaccattgtcaggtaccccagatgacggccatcctccagcatgtgggtggcagcagcaggcatgccaggatgtagcactctccccacctcaatcctttaatgtcttttacatcaacttgttcctgctcagcagttcctacgtcttaagtcatcatgttaacttcaccctaaaactactaggcatgttcagtatctgaggacatgcctaggcagtatttcccagtgacacctccaatggctcctccttatggcatccccagggagccctcccacacccattctcacatctcacagagtcagacctggtaaagtaagagtagtggagaagggtggctctgccactcatctggcatgctatggccgtttcagcaatgcatcctgtaccaccaactgcctggctcctctcgccaaggttattcatgaaaacttcgggatcgtggaagggctaatggtgagtcggggcaggaaggatggcagggaaccaaccccttcttcccaggccttggggcaaggcaaggagcaagcagggagagcagctcaggaaggaaggaagcccatagtggagacaccgtttggagtactttaacaaggttgtagctgtggtggttgccctggttgcctccatatacctcagccacgttctcaagtcctagaagaaagaagaatgagtggttaggaggaaggaggcttcatcttgggtctcttgttccccaagaccacagtccattcctacacagccactcagaagacagtggatgggccatcaaagaaggactggcgaggtggccgcggcgctcaccaaaacatcatcccatcgtccactggggctgccaaggctgtaggcaaagtcatcccagagctcaaagggtatgtagatacaagccaggaaccagggtggacacacaccctaggaaaacctcactggccccaaggcatggggacaatgggagtagaagacctttggctctgactcccacccttactctggaattctccaggaagctaacaggaatggcattccgggtgccaaccccaaacgtgtcagttgtggacctgacctgccgcctggccaagcctgcttcttactcggctatcacggaggctgtgaaagctgcagccaagggacctttggctggcatccttgcttacacagaggaccaggtagggactgaggaaaccttgggggggtgctctggggagggacttgggaaatcccactcaccaggaagctattctgcatgggccaagctgcagaccagctgtccaggtgtttcacacccaaggagttgcaggccagccaggactacacaatgagaccctatctcaaacacagctttccctgcaagcctgggaaagtgggccttcctgcctcggtgtcttgaccagatgtttctttgatctaacgatgctgcttttctgcaccatggtgactggaagtcacttgtgtaacccatagcacatactgtccaagaaaacagcgaactaagagctaaaatggagctttcgctaataattttatatatcaccattgctctgttcagacacaccagaagagagggcatcagaccccattacagatggttgtgagccactgtgtgttgatgggaattgaactcgggacctctggatgagcactcagtgctcttaaccactgagccatctctccggccccagaaggctcttaatacactcctgaacaaaagacagcagttctcattttctggattcacacccaggggccaggccaggtcagacacctttactgaacagactatcttaggtgtggaggcacaggcactcaggtgatagaagcagtagacttctatgaatgtgagggcagccctctatatagtgagttccagagctatatagcaaaccctgtatcaaaaaaaaaaaaaaaaaaaaaaaagtcacctccaccttagtgaagttccctggcatttggcttttacaggtccactctcaaatgccaagatgtcatttgaatgggtcagatcaaatcagccccaatgccaacccacccccaaccccctgtgttcctaaggctacagcccccaagatctgtgtctgtagctccttccctctgctcccaagacccagcctcgtgccagtcatcgcttcatgtaacgtttaatgcaacctcattagggtattgtgatctgtatcctcttcccccgtgtgggtacatgcagatgcacggggcacatgtaccggtacaccaggtacaggtgataaacatgtcttcctccattgcactccacttgttaccgaggcaagatgtaaggtagcttgacaactaactaatctagctagtcatcttgttccaaggagcctcagtctggagctggactgctgggcctataagtgtccatcaaccgcccagcctctatatggattctgcggatccaaacacagaaacgtcacgcttatgtgacaagcacttttatccactgagctcccctgctgtgtccctgagacaaaggttccacctgccttgatcacattctgtcaccaacacaaaaggtctctgtccagtataccagatgagtgactgacaatgtggtttgagagtctgggttcctggagatctttgctcgactacatttatctttgaaattgtcacgcaggtggtctccacggactttaacggcaatccccattcttccatctttgatgctaaggctggaattgccctcaatgacaacttcgtgaagcttgttgcctggtaaaggggaaagatgccagaggatagtgggggaggctgacaagagggcgagcgcgcgagtcgtgacacgtacacacacacacacacacacacacacacacacacacacacgcctggtaaaggggaaagatgccagaggatagtgggggaggctgacaagagggcgagcgcgcgagtgcgtacacgtacacacacacacacacacacacacacacacacacacacacacacacacacacacacacacgcctggtaaaggggatagatgccagaggatagtgggggaggctgacaagagggcgagcgcgcgagtgcgtacacgtatacacacacacacacacacactgacgcctccttcctcgcctcctcaggtacgacaacgaatatggctacagtaaccgagtggtcgacctcctccgctacatgtttagccgagagaagtaacacaaaaggcccctccttgctcccctgcgcacctcgcgttcctgacttcggcttccactcaaaggcgccgccaccgggtcaacaatgaaataaaaacgagaatgcgcacagctttgccctcttgtctctgcgccgatcgagctc

SEQ ID NO: 23 - DNA, 20 bp, Artificial
  PCR primer from the neo resistance gene used in conjunction with SEQ ID NO 24 for detecting targeted GAPDS chromosomes
agcgttggctacccgtgata

SEQ ID NO: 24 - DNA, 24 bp, Mus musculus
cgtgatagccgagtaagaagcagg

(19) United States
(12) Patent Application Publication
(10) Pub. No.: US 2005/0266515 A1
(43) Pub. Date: Dec. 1, 2005

(54) Glyceraldehyde 3-phosphate dehydrogenase-S (GAPDHS), a glycolytic anzyme expressed only in male germ cells, is a target for male contraception

(75) Inventors: Deborah A. O'Brien, Chapell Hill, NC (US); Edward M. Eddy, Chapel Hill, NC (US)

Correspondence Address: JENKINS, WILSON & TAYLOR, P. A., 3100 TOWER BLVD, SUITE 1400, DURHAM, NC, 27707, US

(73) Assignees: The University of North Carolina at Chapel Hill, Chapel Hill, NC (US); The Gov. of the U.S.A as represented by the Secretary of the Dept. of Health & Human Services, Washington, DC (US)

(21) Appl. No.: 11/140,417

(22) Filed: May 27, 2005

Related U.S. Application Data
(63) Continuation-in-part of application No. PCT/US03/37800, filed on Nov. 26, 2003.
(60) Provisional application No. 60/429,638, filed on Nov. 27, 2002.

(51) Int. Cl. A61K 38/54; A61K 31/56; G06F 19/00; G01N 33/48; G01N 33/50; C12Q 1/26

(52) U.S. Cl. 435/25; 514/2; 514/169; 702/19

(57) ABSTRACT

Methods for identifying modulators of a male germ cell-specific glyceraldehyde 3-phosphate dehydrogenase (GAPDHS) are disclosed. Also disclosed are methods for screening potential modulators for an ability to modulate biological functions of a GAPDHS polypeptide.

### CROSS REFERENCE TO RELATED APPLICATIONS

This application is a continuation-in-part of PCT International Patent Application PCT/US2003/037800, filed Nov. 26, 2003, which itself is based on and claims priority to U.S. Provisional Application Ser. No. 60/429,638, filed Nov. 27, 2002, the disclosure of each of which is herein incorporated by reference in its entirety.

### GRANT STATEMENT

This work was supported by a grant from the National Institute of Child Health & Human Development/National Institutes of Health (NICHD/NIH) through cooperative agreement U54 HD35041 as part of the Specialized Cooperative Centers Program in Reproductive Research and by funding for Division of Intramural Research Project ZO1-ES-70076 LRDT, National Institute of Environmental Health Sciences (NIEHS)/NIH. Thus, the U.S. government has certain rights in the presently disclosed subject matter.

### TECHNICAL FIELD

The presently disclosed subject matter relates, in general, to methods and compositions for modulation of reproduction, including but not limited to contraceptive methods and compositions. More particularly, the presently disclosed subject matter relates to a method of screening candidate compositions to determine if they have modulation activity with respect to a male germ cell-specific isoform of glyceraldehyde 3-phosphate dehydrogenase (GAPDH; referred to herein as GAPDHS) and to employing GAPDHS activity-modulating compositions in a method for modulating reproduction, including but not limited to a contraceptive method.

Table of Abbreviations

| | |
|---|---|
| Å | Ångstrom |
| amu | atomic mass unit(s) |
| ATP | adenosine triphosphate |
| CASA | computer-aided sperm analysis |
| cDNA | complementary DNA |
| DNA | deoxyribonucleic acid |
| EC₅₀ | concentration that produces 50% of the |
| | maximum response for a modulator |
| | interacting with a druggable region |
| ES | embryonic stem (cell) |
| G3P | D-glyceraldehyde 3-phosphate |
| GAPDH | glyceraldehyde 3-phosphate |
| | dehydrogenase, generically or somatic |
| | isoform |
| GAPDH | human gene encoding GAPDH |
| Gapdh | mouse or rat gene encoding GAPDH |
| GAPDHS | glyceraldehyde 3-phosphate |
| | dehydrogenase, male germ cell- |
| | specific isoform (human or species |
| | non-specific) |
| GAPDHS | human gene encoding GAPDHS |
| Gapdhs | mouse or rat glyceraldehyde 3-phosphate |
| | dehydrogenase, male germ cell- |
| | specific isoform |
| Gapdhs | mouse or rat gene encoding Gapdhs |
| Gapdhs^{-/-} | mouse in which both alleles of the Gapdhs |
| mouse | gene are mutated |
| GST | glutathione-S-transferase |
| GST-tag | a peptide comprising a recognition |
| | sequence for thrombin protease |
| His-tag | a peptide, usually consisting of about 6 |
| | histidine residues, which can interact |
| | with a coordinated metal ion (e.g. |
| | nickel) |
| IC₅₀ | the concentration of an inhibitor that is |
| | required for 50% inhibition of an |
| | enzyme under a given set of conditions |
| M | molar (moles per liter) |
| Kcal | kilocalories |
| K_{d} | dissociation constant |
| kDa | kilodalton(s) |
| MCT | monocarboxylate transporter |
| NAD | nicotinamide adenine dinucleotide |
| neo | neomycin (G418) resistance |
| PBS | phosphate-buffered saline |
| PCR | polymerase chain reaction |
| RMSD | root mean square deviation |
| RNA | ribonucleic acid |
| SDS | sodium dodecyl sulfate |
| SSC | standard saline citrate; 1x SSC is 0.015 M |
| | NaCl/0.0015 mM sodium citrate/pH 7.0 |
| SV40 | simian virus 40 |
| TCA | tricarboxylic acid |
| T_{d} | dissociation temperature |
| tGAPDHS | a truncated form of a GAPDHS |
| | polypeptide that excludes the N- |
| | terminal proline-rich domain |
| tk | thymidine kinase |
| Tₘ | thermal melting point |
| TPI | triose phosphate isomerase |

Table of Amino Acid Abbreviations and Corresponding mRNA Codons

| Amino Acid | 3-Letter | 1-Letter | mRNA Codons |
|---|---|---|---|
| Alanine | Ala | A | GCA; GCC; GCG; GCU |
| Arginine | Arg | R | AGA; AGG; CGA; |
| | | | CGC; CGG; CGU |
| Asparagine | Asn | N | AAC; AAU |
| Aspartic Acid | Asp | D | GAC; GAU |
| Cysteine | Cys | C | UGC; UGU |
| Glutamic Acid | Glu | E | GAA; GAG |
| Glutamine | Gln | Q | CAA; CAG |
| Glycine | Gly | G | GGA; GGC; GGG; GGU |
| Histidine | His | H | CAC; CAU |
| Isoleucine | Ile | I | AUA; AUC; AUU |
| Leucine | Leu | L | UUA; UUG; CUA; |
| | | | CUC; CUG; CUU |
| Lysine | Lys | K | AAA; AAG |
| Methionine | Met | M | AUG |
| Proline | Pro | P | CCA; CCC; CCG; CCU |
| Phenylalanine | Phe | F | UUC; UUU |
| Serine | Ser | S | ACG; AGU; UCA; |
| | | | UCC; UCG; UCU |
| Threonine | Thr | T | ACA; ACC; ACG; ACU |
| Tryptophan | Trp | W | UGG |
| Tyrosine | Tyr | Y | UAC; UAU |
| Valine | Val | V | GUA; GUC; GUG; GUU |

### BACKGROUND ART

The current world population is in excess of six billion persons, and is growing at an annual rate of over 75 million new inhabitants per year. At this rate, the world's population will reach 10 billion by the year 2050. Since the mid-1980s, however, the rate of food production in the world has increased by only about 1% per year, which is less than the rate of population growth. The inability of new food production to match the rate of population growth has potentially devastating future consequences.

One possibility for addressing these issues is to alter the rate at which the population grows. Life expectancy has increased steadily over the last 50 years, in part due to significant medical advances, and this trend would be expected to continue in the future. Medical advances are also lowering infant mortality rates. The net result of these two factors is that those who are born in the future stand a better chance of surviving to adulthood, and furthermore a higher likelihood of living to an older age. Thus, concerns about the ability to adequately feed, house, and care for the world's population will continue to intensify.

One way to address these concerns is to lower the rate at which children are born, preferably by decreasing the rate at which pregnancy occurs. Contraceptive methods and devices are widely available in developed countries, and attempts are being made to increase the ability of people in developing countries to gain access to such methods and devices. For example, several forms of temporary female contraception are available, including barrier devices, spermicides, and contraceptive pills and implants. Barrier devices prevent either the sperm from reaching the ovum, or alternatively, the implantation in the uterus of the fertilized ovum. Other contraceptive methods interfere with the normal biochemical processes that result in the production of a fertilizable egg. However, particularly with regard to the latter form of contraception, it would likely be much more acceptable to prevent the joining of sperm and egg than to prevent the normal development of an embryo once fertilization has already taken place.

While various temporary contraceptive methods are available for females, male contraception is limited to vasectomy, which is essentially permanent, and the use of barrier devices, which are often less than 100% effective. What is needed, therefore, is a convenient method of temporary male contraception that interferes with the ability of sperm to effectively fertilize an ovum, but is reversible, which would allow the person to reproduce in the future should he so desire.

Thus, there exists a long-felt and continuing need in the art for new methodologies that will allow safe, specific, and reversible male contraception. The presently disclosed subject matter addresses this and other needs in the art.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides methods and compositions for modulating the biological activity of glyceraldehyde 3-phosphate dehydrogenase (GAPDH) enzymes. These enzymes include the somatic isoform of GAPDH and a male germ cell-specific isoform, GAPDHS. In some embodiments, the modulator is selected from the group consisting of the compounds disclosed in Tables 7, 9, and 11. In some embodiments, the modulator is selected from the group consisting of LT_{—}00249157, 11K_{—}064, T0501_{—}7749, T0511_{—}4909, T0501_{—}7933, T0506_{—}9350, and T0515_{—}4928.

In some embodiments, the presently disclosed subject matter provides a method of contraception comprising administering an effective amount of a male germ cell-specific isoform of glyceraldehyde 3-phosphate dehydrogenase (GAPDHS) activity inhibitor to a subject in need thereof.

The presently disclosed subject matter also provides a method of inhibiting sperm motility in a subject in which said inhibition is desired, the method comprising administering an effective amount of a male germ cell-specific isoform of glyceraldehyde 3-phosphate dehydrogenase (GAPDHS) activity inhibitor to the subject. In some embodiments, the inhibitor interacts with one or more of the following residues in human male germ cell-specific isoform of glyceraldehyde 3-phosphate dehydrogenase (GAPDHS): N81, G82, F83, G84, R85, I86, G87, L89, N105, D106, P107, F108, C150, K151, E152, P153, E168, S169, T170, V172, Y173, L174, S175, A178, S193 P197, S223, C224, T225, H251, S252, Y253, T254, A255, T256, Q257, K258, S264, R265, A267, R269, D270, G271, I279, P280, A281, S282, T283, G284, A304, R306, P310, N388, E389, Y392, and S393 of SEQ ID NO: 6. In some embodiments, the inhibitor interacts with one or more of the following residues in mouse male germ cell-specific isoform of glyceraldehyde 3-phosphate dehydrogenase (Gapdhs): N111, G112, F113, G114, R115, I116, L119, N135, D136, P137, F138, C180, K181, D182, P183, E198, C199, T200, V202, Y203, L204, S205, A208, T223, P227, S253, C254, T255, H281, S282, Y283, T284, A285, T286, Q287, K288, S294, K295, D297, R299, G300, G301, I309, P310, S311, S312, T313, G314, A334, R336, P340, N418, E419, Y422, and S423 of SEQ ID NO: 2. In some embodiments, the inhibitor interacts with one or more of the following residues in rat male germ cell-specific isoform of glyceraldehyde 3-phosphate dehydrogenase (Gapdhs): N105, G106, F107, G108, R109, I110, L113, N129, D130, P131, F132, C174, K175, E176, P177, E192, A193, T194, V196, Y197, L198, S199, A202, T217, P221, S247, C248, T249, H275, A276, Y277, T278, A279, T280, Q281, K282, S288, K289, D291, R293, G294, G295, I303, P304, S305, S306, T307, G308, A328, R330, P334, N412, E413, Y416, and S417 of SEQ ID NO: 4.

The presently disclosed subject matter also provides a method of screening a candidate composition for an effect on reproduction. In some embodiments, the method comprises (a) contacting a male germ cell-specific isoform of glyceraldehyde 3-phosphate dehydrogenase (GAPDHS) with a candidate compound; (b) determining an effect of the candidate compound on a biological activity of the GAPDHS; and (c) determining whether the candidate compound has an effective on reproduction based on the effect of the candidate compound on a biological activity of the GAPDHS.

The presently disclosed subject matter also provides a method of screening a candidate composition for an effect on sperm motility. In some embodiments, the method comprises (a) contacting a male germ cell-specific isoform of glyceraldehyde 3-phosphate dehydrogenase (GAPDHS) with a candidate compound; (b) determining an effect of the candidate compound on a biological activity of the GAPDHS; and (c) determining whether the candidate compound has an effect on sperm motility based on the effect of the candidate compound on a biological activity of the GAPDHS.

In some embodiments of the disclosed methods, the candidate compound is screened for selective inhibition of male germ cell-specific isoform of glyceraldehyde 3-phosphate dehydrogenase (GAPDHS) as compared to a somatic glyceraldehyde 3-phosphate dehydrogenase (GAPDH) enzyme. In some embodiments of the disclosed methods, the male germ cell-specific isoform of glyceraldehyde 3-phosphate dehydrogenase (GAPDHS) is a recombinant GAPDHS. In some embodiments of the disclosed methods, the contacting is carried out in vitro. In some embodiments of the disclosed methods, the contacting is carried out by administering the candidate compound to a test subject. In some embodiments of the disclosed methods, the effect is an inhibitory effect.

In some embodiments of the disclosed methods, the male germ cell-specific isoform of glyceraldehyde 3-phosphate dehydrogenase (GAPDHS) is human GAPDHS. In some embodiments, the candidate compound is designed to interact with one or more of the following residues in human male germ cell-specific isoform of glyceraldehyde 3-phosphate dehydrogenase (GAPDHS): N81, G82, F83, G84, R85, I86, G87, L89, N105, D106, P107, F108, C150, K151, E152, P153, E168, S169, T170, V172, Y173, L174, S175, A178, S193 P197, S223, C224, T225, H251, S252, Y253, T254, A255, T256, Q257, K258, S264, R265, A267, R269, D270, G271, I279, P280, A281, S282, T283, G284, A304, R306, P310, N388, E389, Y392, and S393 of SEQ ID NO: 6.

In some embodiments of the disclosed methods, the male germ cell-specific isoform of glyceraldehyde 3-phosphate dehydrogenase (GAPDHS) is mouse Gapdhs. In some embodiments, the candidate compounds is designed to interact with one or more of the following residues in mouse male germ cell-specific isoform of glyceraldehyde 3-phosphate dehydrogenase (Gapdhs): N111, G112, F113, G114, R115, I116, L119, N135, D136, P137, F138, C180, K181, D182, P183, E198, C199, T200, V202, Y203, L204, S205, A208, T223, P227, S253, C254, T255, H281, S282, Y283, T284, A285, T286, Q287, K288, S294, K295, D297, R299, G300, G301, I309, P310, S311, S312, T313, G314, A334, R336, P340, N418, E419, Y422, and S423 of SEQ ID NO: 2.

In some embodiments of the disclosed methods, the male germ cell-specific isoform of glyceraldehyde 3-phosphate dehydrogenase (GAPDHS) is rat Gapdhs. In some embodiments, the inhibitor interacts with one or more of the following residues in rat male germ cell-specific isoform of glyceraldehyde 3-phosphate dehydrogenase (Gapdhs): N105, G106, F107, G108, R109, I110, L113, N129, D130, P131, F132, C174, K175, E176, P177, E192, A193, T194, V196, Y197, L198, S199, A202, T217, P221, S247, C248, T249, H275, A276, Y277, T278, A279, T280, Q281, K282, S288, K289, D291, R293, G294, G295, I303, P304, S305, S306, T307, G308, A328, R330, P334, N412, E413, Y416, and S417 of SEQ ID NO: 4.

The presently disclosed subject matter also provides a method for identifying a male germ cell-specific isoform of glyceraldehyde 3-phosphate dehydrogenase (GAPDHS) modulator. In some embodiments, the method comprises (a) providing atomic coordinates of a GAPDHS to a computerized modeling system; and (b) modeling a ligand that fits spatially into a binding pocket of the GAPDHS to thereby identify a GAPDHS modulator.

The presently disclosed subject matter also provides a method of modeling an interaction between a male germ cell-specific isoform of glyceraldehyde 3-phosphate dehydrogenase (GAPDHS) and a ligand. In some embodiments, the method comprises (a) providing a homology model of a target GAPDHS; (b) providing atomic coordinates of a ligand; and (c) docking the ligand with the homology model to form a GAPDHS/ligand model.

In some embodiments of the disclosed methods, the method further comprises screening for selective inhibition of male germ cell-specific isoform of glyceraldehyde 3-phosphate dehydrogenase (GAPDHS) as compared to a somatic glyceraldehyde 3-phosphate dehydrogenase (GAPDH) enzyme.

The presently disclosed subject matter also provides a method of designing a modulator of a male germ cell-specific isoform of glyceraldehyde 3-phosphate dehydrogenase (GAPDHS). In some embodiments, the method comprises (a) selecting a candidate GAPDHS ligand; (b) determining which amino acid or amino acids of the GAPDHS interact with the ligand using a three-dimensional model of a GAPDHS; (c) identifying in a biological assay for GAPDHS activity a degree to which the ligand modulates the activity of the GAPDHS; (d) selecting a chemical modification of the ligand wherein the interaction between the amino acids of the GAPDHS and the ligand is predicted to be modulated by the chemical modification; (e) synthesizing a ligand having the chemical modified to form a modified ligand; (f) identifying in a biological assay for GAPDHS activity a degree to which the modified ligand modulates the biological activity of the GAPDHS; and (g) comparing the biological activity of the GAPDHS in the presence of modified ligand with the biological activity of the GAPDHS in the presence of the unmodified ligand, whereby a modulator of a GAPDHS is designed. In some embodiments, the method further comprises screening for selective inhibition of male germ cell-specific isoform of glyceraldehyde 3-phosphate dehydrogenase (GAPDHS) as compared to a somatic glyceraldehyde 3-phosphate dehydrogenase (GAPDH) enzyme. In some embodiments, the method further comprises repeating steps (a) through (f) if the biological activity of the male germ cell-specific isoform of glyceraldehyde 3-phosphate dehydrogenase (GAPDHS) in the presence of the modified ligand varies from the biological activity of the GAPDHS in the presence of the unmodified ligand.

The methods and compositions of the presently disclosed subject matter are applicable to any species, and are particularly envisioned to be applicable to mammals. Representative mammals include, but are not limited to humans, mice, and rats.

The methods and compositions of the presently disclosed subject matter take advantage of various interactions between GAPDHS polypeptides and other molecules. In some embodiments, the interactions are selected from the group consisting of a van der Waals interaction, a hydrophobic interaction, hydrogen bonding, and combinations thereof.

### BRIEF DESCRIPTION OF THE DRAWINGS

FIGS. 1A-1D depict an amino acid sequence alignment of mouse Gapdhs (SEQ ID NO: 2), rat Gapdhs (SEQ ID NO: 4), human GAPDHS (SEQ ID NO: 6), and human somatic isoform GAPDH from muscle (SEQ ID NO: 8) that was used for the development of the initial homology structural model.

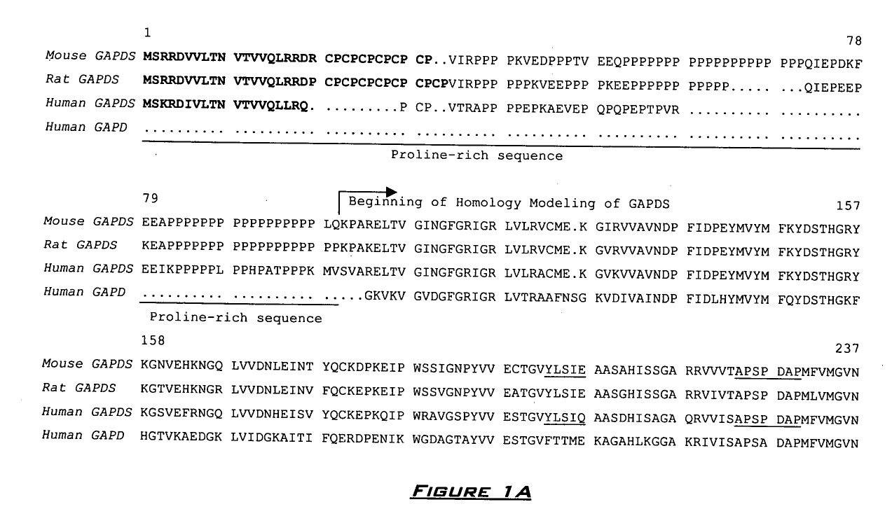

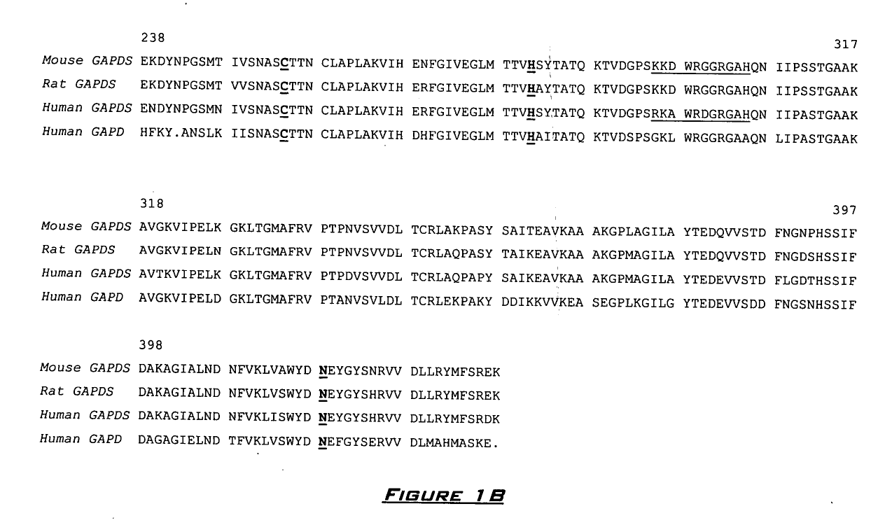

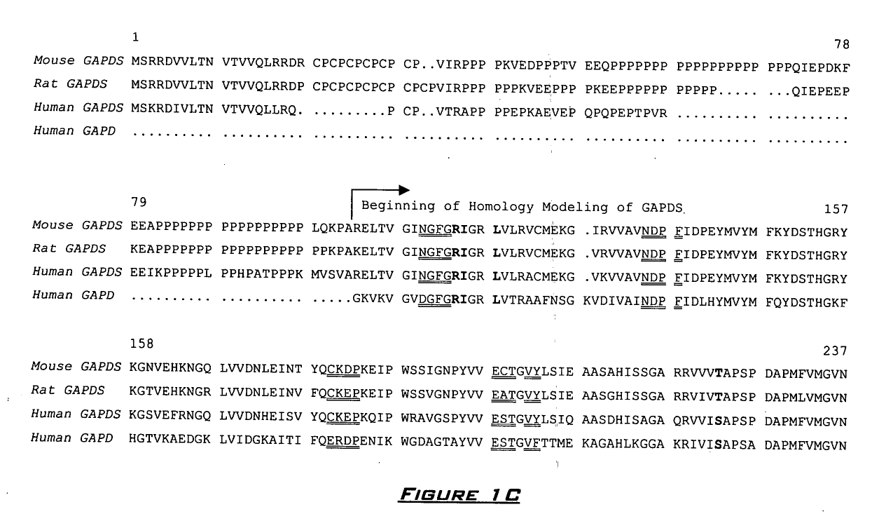

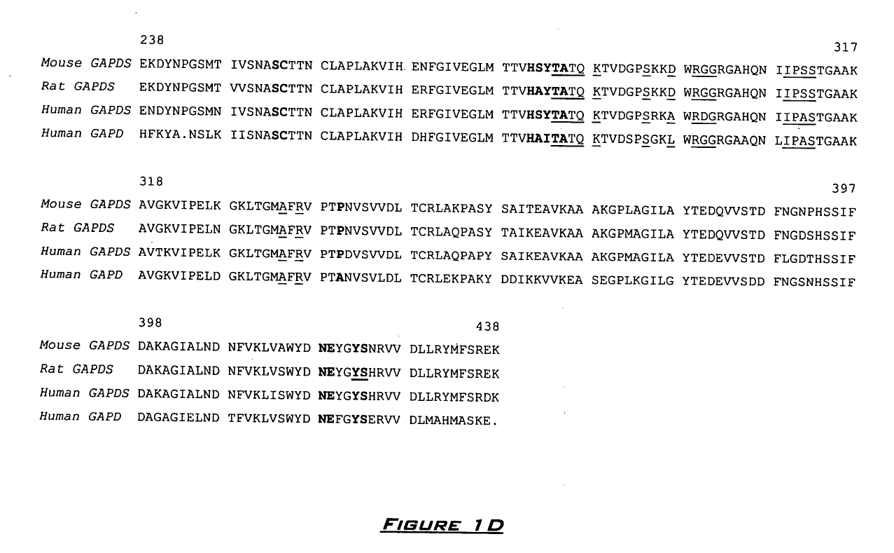

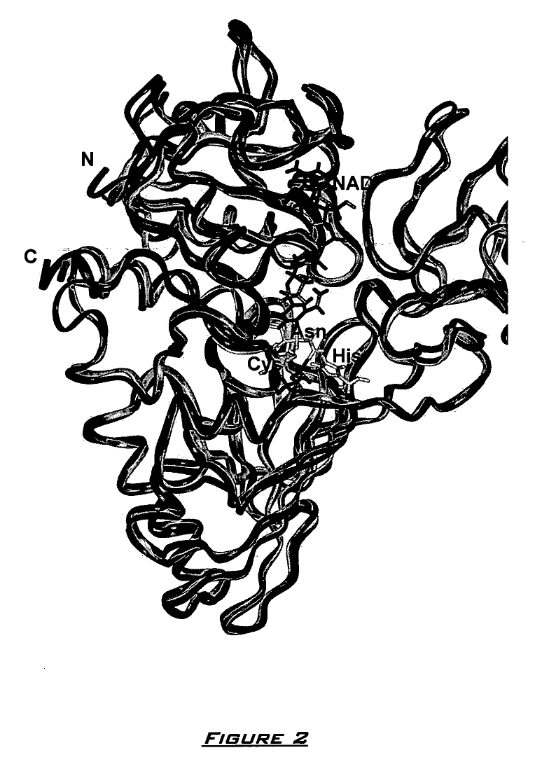

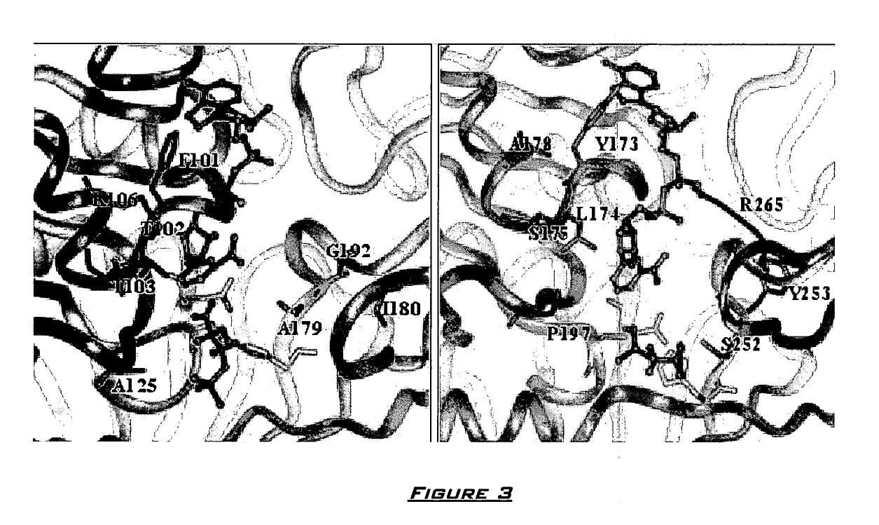

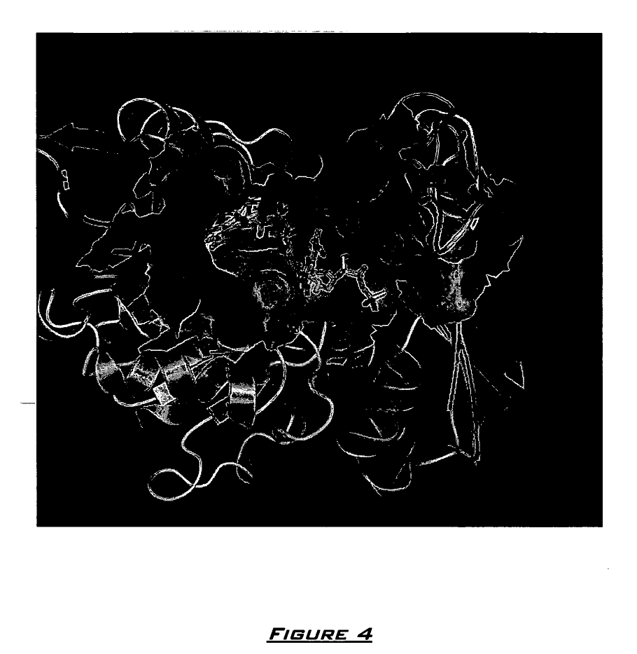

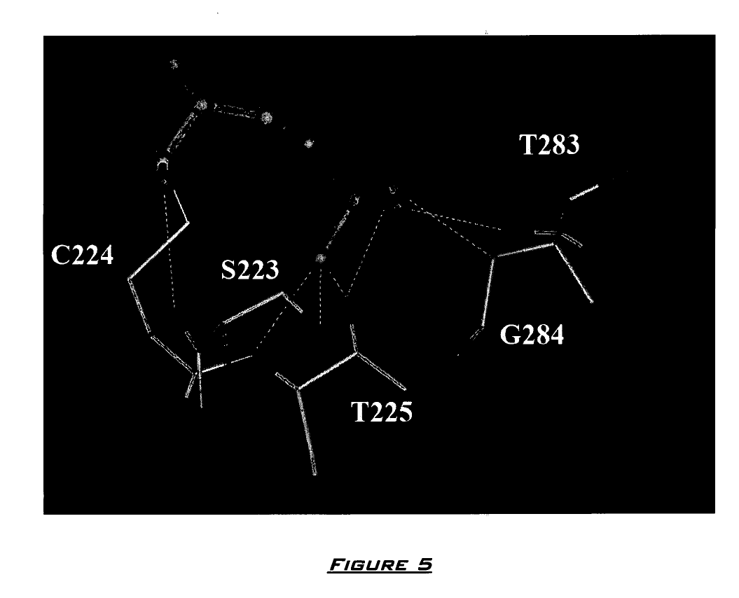

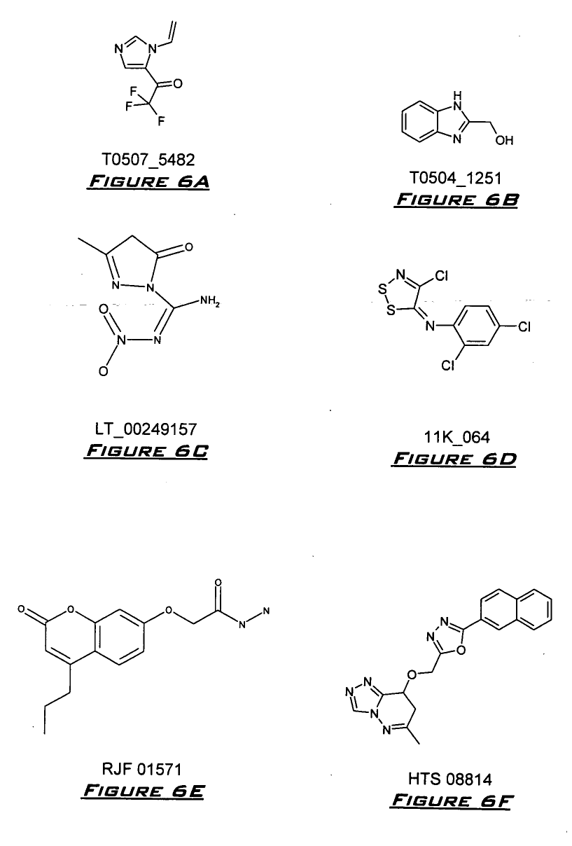

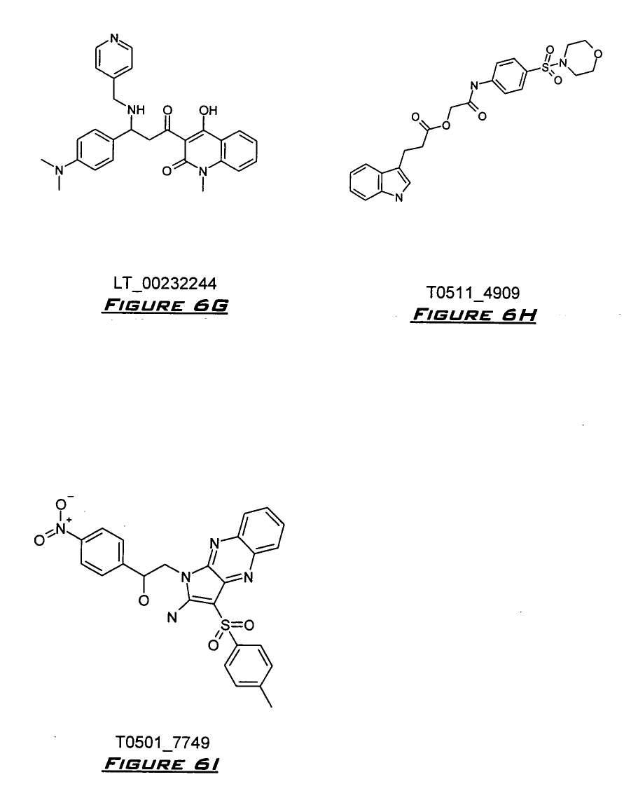

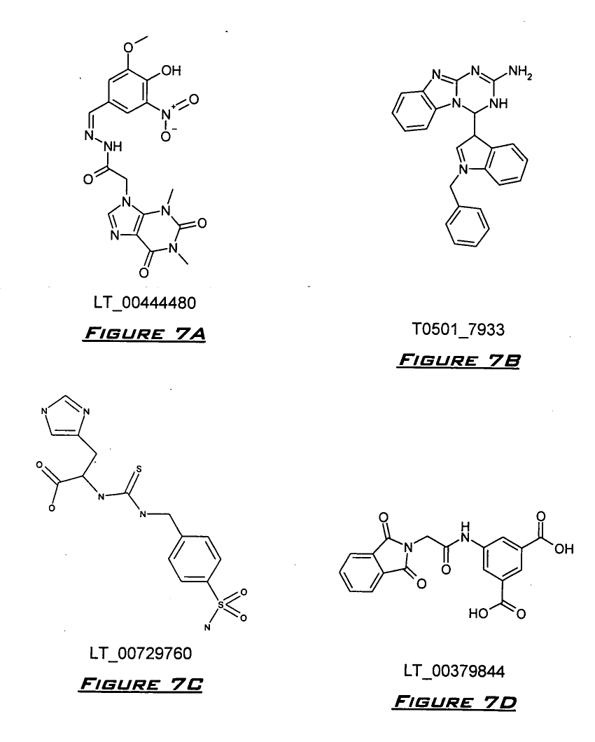

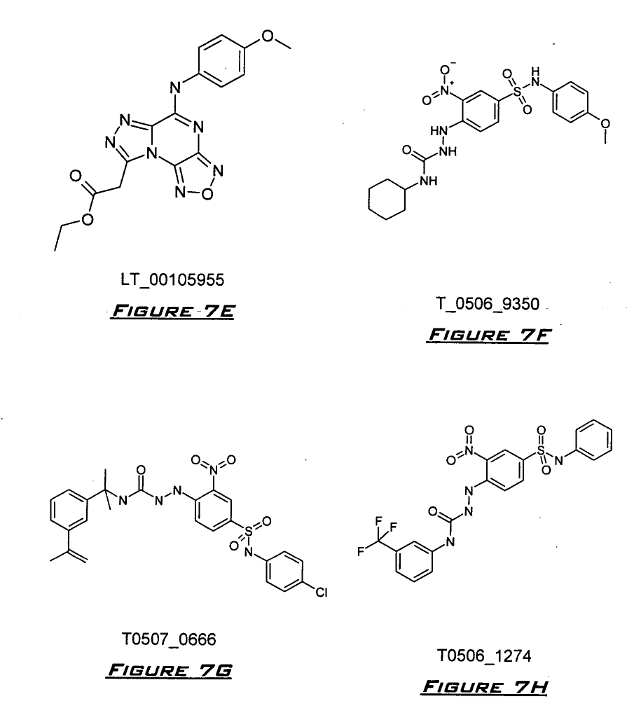

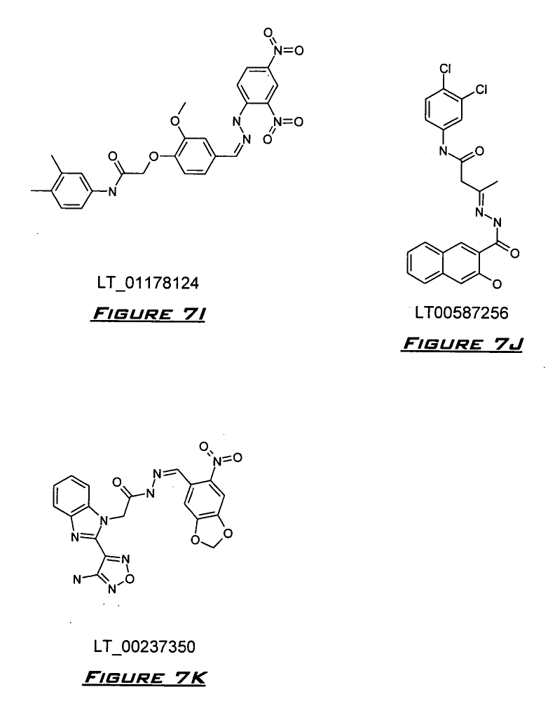

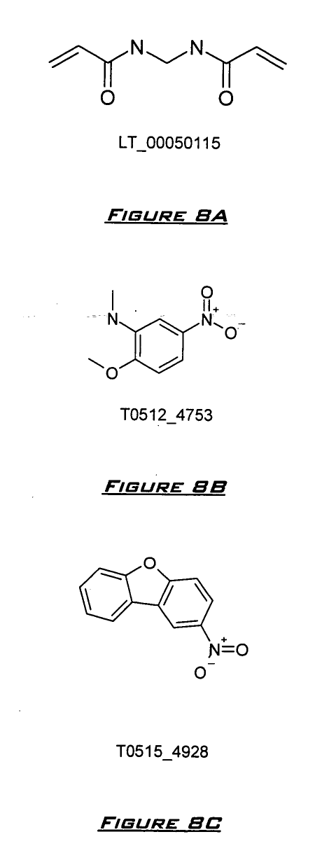

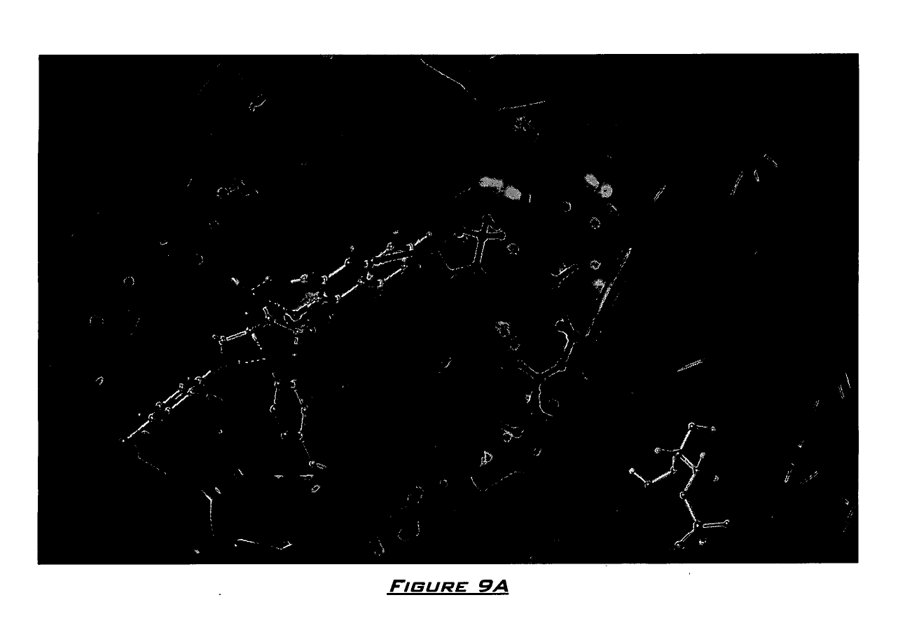

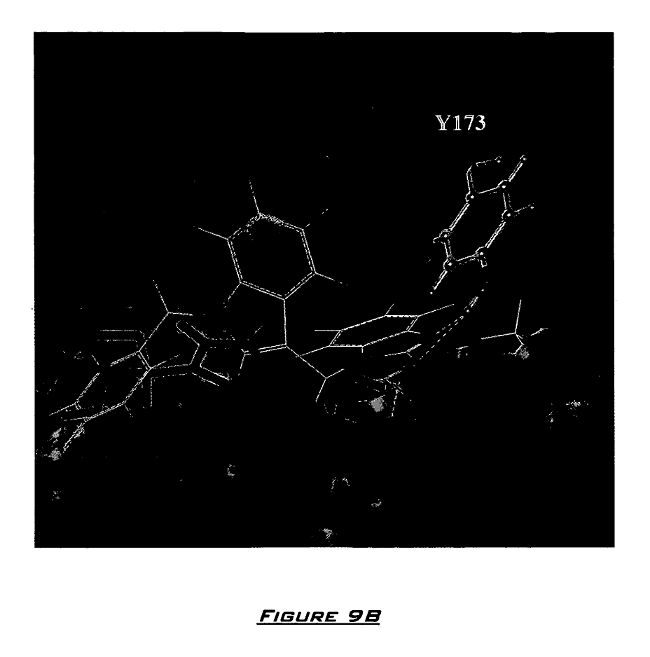

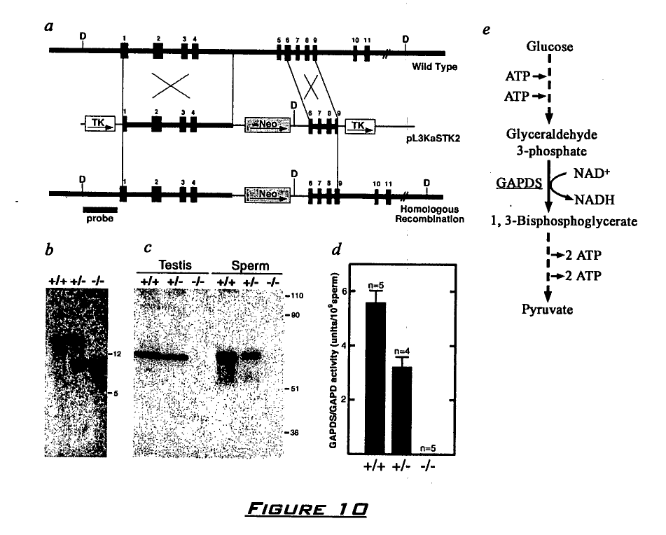

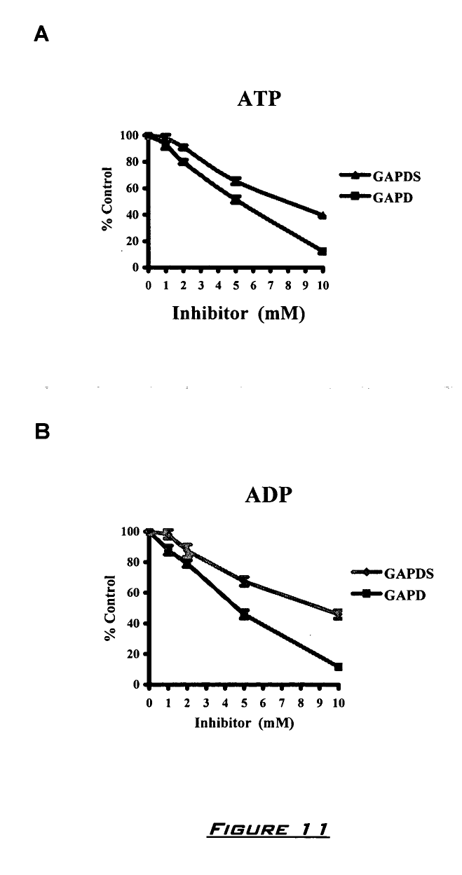

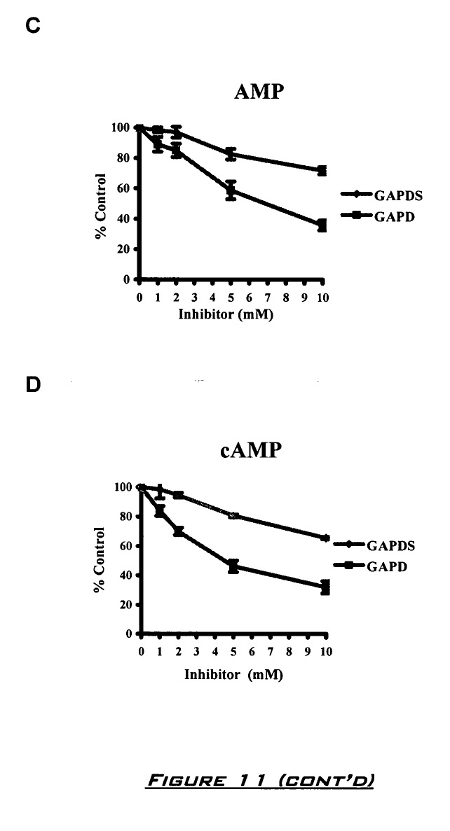

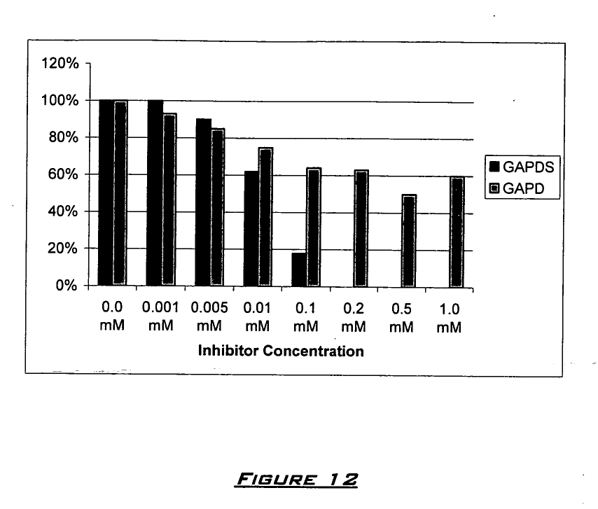

In FIG. 1A, the amino acids in bold at the N-terminus of the mouse, rat, and human GAPDHS sequences correspond to conserved residues present in the N-terminal proline rich domains found in these polypeptides. In FIGS. 1A and 1B, amino acids underlined and in regular typeface correspond to certain protein loops discussed in more detail in Example 3 (SEQ ID NOs: 10, 11, 13, 14, 16, and 18). Amino acids underlined and in bold correspond to the catalytic cysteine, histidine, and asparagines found in GAPDH and GAPDHS. In FIGS. 1C and 1D, amino acids in bold correspond to Pocket 1, amino acids that are double underlined correspond to Pocket 2, and amino acids that are single underlined correspond to Pocket 3, the latter of which partially overlaps with Pocket 1 (thus some amino acids are bolded and single underlined).

FIG. 2 depicts a ribbon superimposition of GAPDH, human GAPDHS, and mouse Gapdhs (grey, black, and blue, respectively), with no side chains depicted. NAD and G3P are shown in stick form in red (NAD is above G3P in the Figure), and the location of the catalytic Cys-His-Asn residues from human muscle (Nagradova, 2001) are shown in stick form in yellow. The N and C termini are labeled.

FIG. 3 depicts the active sites of the somatic form of human GAPDH (left panel) and the male germ cell-specific GAPDHS (right panel) with NAD and G3P bound to each. NAD and G3P (depicted in red) are indicated in ball-and-stick form, with NAD towards the middle top of each panel and G3P at the middle bottom. Also shown in this Figure are the locations of eight amino acids (with side chains depicted in green) that differ between the two isoforms: namely, F101, T102, T103, K106, A125, A179, 1180, and G192 in human GAPDH, which are replaced by Y173, L174, S175, A178, P197, S252, Y253, and R265, respectively, in human GAPDHS. The catalytic Cys-His-Asn residues are shown in stick form in yellow.

FIG. 4 depicts a model of the human GAPDHS sperm enzyme based on the high-resolution crystal structure of Gapdh from the South China sea lobster (Shen et al., 2000). Using the SYBYL® Site ID program (available from Tripos, Inc., St. Louis, Mo., United States of America), the surface of the active site was divided into three pockets, which are shown in cyan (Pocket 1), yellow (Pocket 2), and pink (Pocket 3). Pocket 1 includes both the substrate and nicotinamide moiety of the NAD cofactor. The adenine moiety of the cofactor binds in Pocket 2. Pocket 3 covers the binding site for inorganic phosphate, which is required for the addition of a second phosphate group to the G3P substrate during catalysis. The G3P substrate is shown in orange and the NAD cofactor in green. Differences between the sperm and somatic isozymes are highlighted in dark blue.

FIG. 5 depicts amino acids that comprise Small Pocket 1 for human GAPDHS (stick form, with the amino and carboxyl groups shown in blue and red, respectively.). This pocket includes amino acids that can form hydrogen bonds (represented by dashed lines) with the G3P substrate (ball-and-stick form at the top).

FIGS. 6A-6I depict the structures of nine inhibitors listed in Tables 6 and 7 that have been identified by the methods disclosed herein to bind to Pocket 1 and Small Pocket 1 of human GAPDHS.

FIGS. 7A-7K depict the structures of the eleven inhibitors listed in Tables 8 and 9 that have been identified by the methods disclosed herein to bind to Pocket 2 of human GAPDHS.

FIGS. 8A-8C depict the structures of the three inhibitors listed in Table 11 that have been identified by the methods disclosed herein to bind to Pocket 3 of human GAPDHS.

FIGS. 9A and 9B depict partial space-filling models of representative inhibitors bound to human GAPDHS.

FIG. 9A depicts a partial space-filling model of inhibitor LT_{—}00587256 bound to human GAPDHS. The inhibitor is shown in ball and stick form, while Pocket 2 is shown in space-filling form (green). Residues of GAPDHS outside of the pocket are depicted in ribbon form (red). The blue ball-and-stick form is NAD, and the yellow ball-and-stick form is G3P. The dashed lines indicate sites of interaction between the inhibitor and the Tyr 173 of the GAPDHS polypeptide. FIG. 9B depicts a partial space-filling model of inhibitor T0501_{—}7933 bound to Pocket 2 of human GAPDHS (green, space-filling form). Here the inhibitor is shown in stick form (thin white lines) with hydrogen bonds to Tyr 173 (ball-and-stick form) indicated by dashed lines. NAD is shown in blue stick form.

FIGS. 10A-10E depict the targeted disruption of the mouse Gapdhs gene.

FIG. 10A depicts a map of the mouse Gapdhs locus and diagrams showing the strategy employed for targeted disruption of the Gapdhs gene. Filled boxes indicate exons. Dra I sites (D) are shown. The bar in the bottom margin indicates the position of a probe used for Southern analysis. FIG. 10B depicts a representative Southern blot used to genotype cells and animals. Genomic DNA form wild type (+/+), heterozygous (+/−) or homozygous (−/−) mutant for Gapdhs gene was digested by Dra I and analyzed by Southern blotting. The probe indicated in panel A detects a Dra I fragment of about 20 kilobases (kb) on a wild type chromosome, and a fragment of about 8 kb on a targeted (i.e. mutant) chromosome. The sizes in kb of DNA standards are shown on the right margin. FIG. 10C depicts Gapdhs protein expression. Testis and sperm proteins from wild type and mutant males were analyzed by Western blotting using an anti-Gapdhs antibody. The sizes in kilodaltons (kDa) of protein standards are shown on the right margin. FIG. 10D depicts an analysis of enzyme activity. Gapdhs/Gapdh enzyme activity in sperm was measured for wild type and mutant males. Values shown are mean±standard error of the mean (s.e.m.). FIG. 10E depicts an abbreviated glycolysis diagram. One molecule of glucose is converted to two molecule of pyruvate by glycolysis, with net production of two ATP molecules in steps followed by GAPDHS/GAPDH reaction.

FIGS. 11A-11D depict the results of testing several competitive inhibitors of NAD cofactor-binding (ATP, ADP, AMP, cyclic AMP) on a truncated mouse Gapdhs polypeptide (orange) and a recombinant mouse Gapdh polypeptide (blue).

FIG. 12 depicts concentration-dependent inactivation of GAPDHS and GAPDH by T0501_{—}7933.

### BRIEF DESCRIPTION OF THE SEQUENCE LISTING

SEQ ID NOs: 1 and 2 are the nucleic acid and deduced amino acid sequences of a mouse Gapdhs cDNA and deduced polypeptide (GENBANK® Accession Nos. NM_{—}008085 and NP_{—}03211), respectively.

SEQ ID NOs: 3 and 4 are the nucleic acid and deduced amino acid sequences of a rat Gapdhs cDNA and deduced polypeptide (GENBANK® Accession Nos. NM_{—}023964 and NP_{—}076454), respectively.

SEQ ID NOs: 5 and 6 are the nucleic acid and deduced amino acid sequences of a human GAPDHS cDNA and deduced polypeptide (GENBANK® Accession Nos. BC036373 and AAH36373), respectively.

SEQ ID NOs: 7 and 8 are the nucleic acid and deduced amino acid sequences of a somatic isoform human GAPDH cDNA and deduced polypeptide (GENBANK® Accession Nos. BC009081 and P00354), respectively.

SEQ ID NO: 9 is an amino acid sequence of a highly conserved domain in somatic and spermatogenic cell isoforms of glyceraldehyde 3-phosphate dehydrogenase that contains the catalytic cysteine (corresponds to residues 251-259 in SEQ ID NO: 2, residues 221-229 in SEQ ID NO: 6, and residues 148-156 in SEQ ID NO: 8).

SEQ ID NOs: 10-12 are the amino acid sequences of a loop in mouse Gapdhs (residues 293-305 of SEQ ID NO: 2), human GAPDHS (residues 263-275 of SEQ ID NO: 6), and human somatic isoform GAPDH (residues 190-202 of SEQ ID NO: 8), respectively, near the substrate-binding pocket of each polypeptide.

SEQ ID NOs: 13-15 are the amino acid sequences of a loop in the NAD cofactor-binding pocket of mouse Gapdhs (residues 203-209 of SEQ ID NO: 2), human GAPDHS (residues 173-179 of SEQ ID NO: 6), and human somatic isoform GAPDH (residues 101-106 of SEQ ID NO: 8), respectively, that differ between the somatic and male germ cell-specific isoforms of GAPDH.

SEQ ID NOs: 16 and 17 are the amino acid sequences of a loop that differs between mouse Gapdhs and human GAPDHS on the one hand, and human somatic isoform GAPDH on the other.

SEQ ID NOs: 18 and 19 are the amino acid sequences of another loop that differs between mouse Gapdhs and human GAPDHS on the one hand, and human somatic isoform GAPDH on the other.

SEQ ID NO: 20 is the amino acid sequence of a highly conserved N-terminal domain of a Gapdhs polypeptide from the mouse and from the rat (corresponds to amino acids 1-19 of SEQ ID NOs: 2 and 4, respectively).

SEQ ID NO: 21 is the amino acid sequence of a highly conserved N-terminal domain of a GAPDHS polypeptide from the human (corresponds to amino acids 1-19 of SEQ ID NO: 6).

SEQ ID NO: 22 is a nucleotide sequence derived from the mouse Gapdhs locus including the complete coding sequence (GENBANK® Accession No. U09964).

SEQ ID NOs: 23 and 24 are the sequences of primers used to detect murine embryonic stem (ES) cells that contained a targeted disruption of the Gapdhs gene.

### DETAILED DESCRIPTION

I. General Considerations

Glyceraldehyde 3-phosphate dehydrogenase (GAPDH) is a nicotinamide adenine dinucleotide (NAD)-dependent enzyme in the glycolytic pathway that reversibly catalyzes the oxidation and phosphorylation of D-glyceraldehyde 3-phosphate (G3P) to 1,3-bisphosphoglycerate. This enzyme occupies a key transition point in the glycolytic pathway between the first phase that consumes ATP and the second phase that produces ATP. While GAPDH is present in most cells, a spermatogenic cell isoform of this enzyme has been identified that is encoded by genes transcribed only in the testis in mouse (Gapdhs; Welch et al., 1992; Welch et al., 1995; SEQ ID NOs: 1 and 2) and human (GAPDHS; Welch et al., 2000; SEQ ID NO: 5 and 6). In addition, a rat Gapdhs cDNA sequence has been reported (GENBANK® Accession No. NM_{—}023964; SEQ ID NOs: 3 and 4) and northern blot analysis suggested that orthologues are present in other mammals (Welch et al., 1992).

The mouse Gapdhs gene is expressed during the latter part of spermatogenesis, with Gapdhs transcription beginning in round spermatids (Welch et al., 1992; Mori et al., 1992) and Gapdhs protein synthesis beginning several days later in condensing spermatids (Bunch et al., 1998). Gapdh was not detected in isolated round or condensing spermatids (Bunch et al., 1998), indicating that the Gapdh gene is down regulated during spermatogenesis. Furthermore, Gapdhs was not detected in mouse or human sperm (Bunch et al., 1998; Welch et al., 2000). Glucose is required for hyperactivated motility of sperm in mice (Fraser & Quinn, 1981; Cooper, 1984) and for fertilization in vitro in mice and humans (Hoppe, 1976; Hoshi et al., 1991; Mahadevan et al., 1997).

Glycolysis appears to be a major source of energy production for fertilization because lactate or pyruvate cannot substitute for glucose as an energy substrate and inhibition of oxidative phosphorylation does not block fertilization in vitro (Fraser & Quinn, 1981).

GAPDHS and GAPDHS have higher molecular mass than mouse or human GAPDH due to proline-rich domains at the N-terminus (Welch et al., 1992; Bunch et al., 1998; Welch et al., 2000). In addition, GAPDHS and Gapdhs have greater sequence identity (83%) than Gapdhs and Gapdh in the mouse (71%) or GAPDHS and GAPDH in the human (68%), excluding the proline-rich N-terminus. Gapdhs is 30 amino acids longer than GAPDHS, largely due to the presence of more prolines in the N-terminal proline-rich segment. However, this segment is not required for enzymatic function and was proposed to bind the enzyme to the sperm's fibrous sheath (Bunch et al., 1998; Welch et al., 2000). In addition, 7 of 8 residues important for NAD cofactor-binding are identical. The eighth residue in the NAD cofactor-binding pocket is a tyrosine in Gapdhs (Y203) and GAPDHS (Y173) instead of a phenylalanine in somatic GAPDH in mouse, human, and other species (Welch et al., 1992).

II. Definitions

For convenience, certain terms employed in the specification, examples, and appended claims are collected here. Unless defined otherwise, all technical and scientific terms used herein have the same meaning as commonly understood by one of ordinary skill in the art to which the presently disclosed subject matter belongs.

Following long-standing patent law convention, the terms “a” and “an” refer to “one or more” when used in this application, including the claims. Thus, the articles “a” and “an” are used herein to refer to one or to more than one (i.e., to at least one) of the grammatical object of the article. By way of example, “an element” refers to one element or more than one element.

As used herein, the term “about”, when referring to a value or to an amount of mass, weight, time, volume, concentration, or percentage, is meant to encompass variations of in some embodiments ±20%, in some embodiments ±10%, in some embodiments ±5%, in some embodiments 1%, and in some embodiments ±0.1% from the specified amount, as such variations are appropriate to perform the disclosed method.

As used herein, the terms “agonist” and “activator” are used interchangeably and refer to an agent that supplements or potentiates the bioactivity of a functional GAPDHS gene or protein.

As used herein, the terms “α-helix” and “alpha-helix” refer to the conformation of a polypeptide chain wherein the polypeptide backbone is wound around the long axis of the molecule in a left-handed or right-handed direction, and the R groups of the amino acids protrude outward from the helical backbone, wherein the repeating unit of the structure is a single turnoff the helix, which extends about 0.56 nm along the long axis.

As used herein, the terms “amino acid” and “amino acid residue” are used interchangeably and refer to any of the twenty naturally occurring amino acids, as well as analogs, derivatives, and congeners thereof; amino acid analogs having variant side chains; and all stereoisomers of any of any of the foregoing. Thus, the term “amino acid” is intended to embrace all molecules, whether natural or synthetic, which include both an amino functionality and an acid functionality and capable of being included in a polymer of naturally occurring amino acids.

An amino acid is formed upon chemical digestion (hydrolysis) of a polypeptide at its peptide linkages. The amino acid residues described herein are in some embodiments in the “L” isomeric form. However, residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxy terminus of a polypeptide. In keeping with standard polypeptide nomenclature abbreviations for amino acid residues are shown in tabular form presented hereinabove.

It is noted that all amino acid residue sequences represented herein by formulae have a left-to-right orientation in the conventional direction of amino terminus to carboxy terminus. In addition, the phrases “amino acid” and “amino acid residue” are broadly defined to include modified and unusual amino acids.

Furthermore, it is noted that a dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

As used herein, the term “antagonist” and “inhibitor” are used interchangeably and refer to an agent that decreases or inhibits the bioactivity of a functional GAPDHS gene or protein.

As used herein, the terms “β-sheet” and “beta-sheet” refer to the conformation of a polypeptide chain stretched into an extended zigzag conformation. Portions of polypeptide chains that run “parallel” all run in the same direction. Portions of polypeptide chains that run “antiparallel” run in opposite directions from each other.

The term “binding” refers to an association, which can be a stable association, between two molecules, e.g., between a polypeptide of the presently disclosed subject matter and a binding partner, due to, for example, electrostatic, hydrophobic, ionic, and/or hydrogen-bond interactions under particular conditions.

As used herein, the term “biological activity” refers to any observable effect flowing from interaction between an enzyme (e.g. a GAPDHS) and a ligand (e.g., a substrate or a product). Representative, but non-limiting, examples of biological activities in the context of the presently disclosed subject matter include, but are not limited to effects on ATP production via glycolysis, sperm motility, and the ability to successfully fertilize an ovum.

As used herein, the terms “candidate substance”, “candidate compound”, “test substance”, and “test compound” are used interchangeably and refer to a substance that is believed to interact with another moiety, for example a given ligand that is believed to interact with a complete GAPDHS polypeptide or a fragment thereof, and which can be subsequently evaluated for such an interaction. Representative candidate substances or compounds include “xenobiotics”, such as drugs and other therapeutic agents, carcinogens and environmental pollutants, natural products and extracts, as well as “endobiotics”, such as steroids, fatty acids, and prostaglandins. Other examples of candidate compounds that can be investigated using the methods of the presently disclosed subject matter include, but are not restricted to, agonists and antagonists of a GAPDHS polypeptide, toxins and venoms, viral epitopes, hormones (e.g., opioid peptides, steroids, etc.), hormone receptors, peptides, enzymes, enzyme substrates, cofactors, lectins, sugars, oligonucleotides or nucleic acids, oligosaccharides, proteins, small molecules, and monoclonal antibodies.

Additionally, the terms “candidate substance”, “candidate compound”, “test substance”, and “test compound” refer to a molecule to be tested by one or more screening method(s) as a putative modulator of a polypeptide of the presently disclosed subject matter or other biological entity or process. A test compound is usually not known to bind to a target of interest. The term “control test compound” refers to a compound known to bind to the target (e.g., a known agonist, antagonist, partial agonist or inverse agonist).

The term “test compound” does not include a chemical added as a control condition that alters the function of the target to determine signal specificity in an assay. Such control chemicals or conditions include chemicals that 1) nonspecifically or substantially disrupt protein structure (e.g., denaturing agents (e.g., urea or guanidinium), chaotropic agents, sulfhydryl reagents (e.g., dithiothreitol and β-mercaptoethanol), and proteases); 2) generally inhibit cell metabolism (e.g., mitochondrial uncouplers); and 3) non-specifically disrupt electrostatic or hydrophobic interactions of a protein (e.g., high salt concentrations, or detergents at concentrations sufficient to non-specifically disrupt hydrophobic interactions). Further, the term “test compound” also does not include compounds known to be unsuitable for a therapeutic use for a particular indication due to toxicity of the subject. In some embodiments, various predetermined concentrations of test compounds are used for screening, such as 0.001 mM, 0.003 mM, 0.01 mM, 0.1 mM, 1.0 mM, 10.0 mM, and various concentrations there between. Examples of test compounds include, but are not limited to, peptides, nucleic acids, carbohydrates, and small molecules. The term “novel test compound” refers to a test compound that is not in existence as of the filing date of this application. In certain assays using novel test compounds, the novel test compounds comprise at least about 50%, 75%, 85%, 90%, 95% or more of the test compounds used in the assay or in any particular trial of the assay.

In some embodiments, the term “candidate composition” as used herein describes any molecule, e.g. protein or pharmaceutical, with the capability of affecting GAPDHS biological activity. Generally, pluralities of assay mixtures are run in parallel with different candidate composition concentrations to obtain a differential response to the various concentrations. Typically, one of these concentrations serves as a negative control, i.e. at zero concentration or below the level of detection.

Candidate compositions encompass numerous chemical classes, though typically they are organic molecules, in some embodiments small organic compounds having a molecular mass of more than 50 and less than about 2,500 daltons, as can be, in some embodiments, encompassed by the term “small molecule” as set forth herein. Candidate compositions comprise functional groups necessary for structural interaction with proteins, particularly hydrogen bonding, and in some embodiments include at least an amine, carbonyl, hydroxyl, or carboxyl group, in some embodiments at least two of the functional chemical groups. The candidate compositions often comprise cyclical carbon or heterocyclic structures and/or aromatic or polyaromatic structures substituted with one or more of the above functional groups. Candidate compositions are also found among biomolecules including, but not limited to peptides, saccharides, fatty acids, steroids, purines, pyrimidines, derivatives, structural analogs, or combinations thereof. In some embodiments, the candidate composition comprises a cofactor scaffold, e.g. an adenine scaffold. In some embodiments, a substrate analog is employed as scaffold.

Candidate compositions are obtained from a wide variety of sources including libraries of synthetic or natural compounds. For example, numerous approaches are available for random and directed synthesis of a wide variety of organic compounds and biomolecules, including expression of randomized oligonucleotides and oligopeptides. Alternatively, libraries of natural compounds in the form of bacterial, fungal, plant, and animal extracts are available or readily produced. Additionally, natural or synthetically produced libraries and compounds are readily modified through conventional chemical, physical, and biochemical approaches, and can be used to produce combinatorial libraries. Known pharmacological agents can be subjected to directed or random chemical modifications, such as acylation, alkylation, esterification, amidification, etc., to produce structural analogs.

As used herein, the term “complementary DNA (cDNA)” refers to a single-stranded DNA molecule that is formed from an mRNA template by the enzyme reverse transcriptase. Typically, a primer complementary to portions of mRNA is employed for the initiation of reverse transcription. Those of ordinary skill in the art also use the term “cDNA” to refer to a double-stranded DNA molecule consisting of such a single-stranded DNA molecule and its complementary DNA strand together in a double-stranded configuration.

The term “complex” refers to an association between at least two moieties (e.g. chemical or biochemical) that have an affinity for one another. Examples of complexes include associations between antigen/antibodies, lectin/avidin, target polynucleotide/probe oligonucleotide, antibody/anti-antibody, receptor/ligand, enzyme/ligand, polypeptide/polypeptide, polypeptide/polynucleotide, polypeptide/cofactor, polypeptide/substrate, polypeptide/inhibitor, polypeptide/small molecule, and the like. “Member of a complex” refers to one moiety of the complex, such as an antigen or ligand. “Protein complex” or “polypeptide complex” refers to a complex comprising at least one polypeptide.

As used herein, the terms “cells”, “host cells”, and “recombinant host cells” are used interchangeably and refer not only to the particular subject cell, but also to the progeny or potential progeny of such a cell. Because certain modifications can occur in succeeding generations due to either mutation or environmental influences, such progeny might not, in fact, be identical to the parent cell, but are still included within the scope of the term as used herein.

As used herein, the terms “chimeric protein” and “fusion protein” are used interchangeably and refer to a fusion of a first amino acid sequence encoding a GAPDH (e.g. GAPDHS) polypeptide with a second amino acid sequence defining a polypeptide domain foreign to, and not homologous with, any domain or sequence of a GAPDH (e.g. GAPDHS) polypeptide. A chimeric protein can present a foreign domain that is found in an organism that also expresses the first protein, or it can be an “interspecies” or “intergenic” fusion of protein structures expressed by different kinds of organisms. In some embodiments, a chimeric protein or a fusion protein can be represented by the general formula X—GAPDH—Y, wherein GAPDH represents a portion of the protein which is derived from a GAPDH polypeptide, and X and Y are independently absent or represent amino acid sequences which are not related to a GAPDH sequence in an organism, including naturally occurring mutants. For example, a fusion protein can comprise amino acid sequences of a transit peptide joined with an amino acid sequence of at least part of a GAPDH polypeptide. As another example, a fusion protein can comprise at least part of a GAPDH amino acid sequence fused with a polypeptide that binds an affinity matrix. Such fusion proteins can be useful for isolating large quantities of GAPDH protein with affinity chromatography. The term “chimeric gene” refers to a nucleic acid construct that encodes a “chimeric protein” or “fusion protein” as defined herein.

Thus, in many examples of fusion proteins, there are two different polypeptide sequences, and in certain cases, there can be more. The sequences can be linked in frame. A fusion protein can include a domain that is found (albeit in a different protein) in an organism that also expresses the first protein, or it can be an “interspecies”, “intergenic”, etc. fusion expressed by different species of organisms. In some embodiments, the fusion polypeptide can comprise one or more amino acid sequences linked to a first polypeptide. In the case where more than one amino acid sequence is fused to a first polypeptide, the fusion sequences can be multiple copies of the same sequence, or alternatively, can be different amino acid sequences. The fusion polypeptides can be fused to the N-terminus, the C-terminus, or the N- and C-terminus of the first polypeptide. Exemplary fusion proteins include polypeptides comprising a glutathione S-transferase tag (GST-tag), histidine tag (His-tag), an immunoglobulin domain, or an immunoglobulin-binding domain.

The term “conserved residue refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz & Schirmer, 1979). According to such analyses, groups of amino acids can be defined where amino acids within a group exchange preferentially with each other, and therefore resemble each other most in their impact on the overall protein structure (Schulz & Schirmer, 1979). One example of a set of amino acid groups defined in this manner include: (i) a charged group, consisting of Glu and Asp, Lys, Arg and His; (ii) a positively-charged group, consisting of Lys, Arg and His; (iii) a negatively-charged group, consisting of Glu and Asp; (iv) an aromatic group, consisting of Phe, Tyr and Trp; (v) a nitrogen ring group, consisting of His and Trp; (vi) a large aliphatic nonpolar group, consisting of Val, Leu and Ile; (vii) a slightly-polar group, consisting of Met and Cys; (viii) a small-residue group, consisting of Ser, Thr, Asp, Asn, Gly, Ala, Glu, Gin and Pro; (ix) an aliphatic group consisting of Val, Leu, Ile, Met and Cys; and (x) a small hydroxyl group consisting of Ser and Thr. Table 1 presents a non-limiting grouping of amino acids that can be considered for performing conservative amino acid substitutions.

TABLE 1
Representative Conservative Amino Acid Substitutions

| Amino Acid Property | Amino Acid |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

As used herein, the term “detecting” refers to confirming the presence of a target entity by observing the occurrence of a detectable signal, such as a radiologic or spectroscopic signal that will appear exclusively in the presence of the target entity.

The term “domain”, when used in connection with a polypeptide, refers to a specific region within such polypeptide that comprises a particular structure or mediates a particular function. In some embodiments, a domain of a polypeptide of the presently disclosed subject matter is a fragment of the polypeptide. In some embodiments, a domain is a structurally stable domain, as evidenced, for example, by mass spectroscopy, or by the fact that a modulator can bind to a druggable region of the domain.

As used herein, the term “DNA segment” refers to a DNA molecule that has been isolated free of total genomic DNA of a particular species. In some embodiments, a DNA segment encoding a GAPDHS polypeptide refers to a DNA segment that comprises a full length polypeptide, but can optionally comprise fewer or additional nucleic acids, yet is isolated away from, or purified free from, total genomic DNA of a source species, such as Homo sapiens. Included within the term “DNA segment” are DNA segments and smaller fragments of such segments, and also recombinant vectors, including, for example, plasmids, cosmids, phages, viruses, and the like.

As used herein, the term “DNA sequence encoding a GAPDHS polypeptide” can refer to one or more coding sequences within a particular individual. Moreover, certain differences in nucleotide sequences can exist between individual organisms, which are called alleles. It is possible that such allelic differences might or might not result in differences in the amino acid sequence of the encoded polypeptide yet still encode a protein with the same biological activity. As is well known, genes for a particular polypeptide can exist in single or multiple copies within the genome of an individual. Such duplicate genes can be identical or can have certain modifications, including nucleotide substitutions, additions, or deletions, all of which still code for polypeptides having substantially the same activity.

The term “druggable region”, when used in reference to a polypeptide, nucleic acid, complex and the like, refers to a region of the molecule that is a target or is a likely target for binding a modulator. For a polypeptide, a druggable region generally refers to a region wherein several amino acids of a polypeptide would be capable of interacting with a modulator or other molecule. For a polypeptide or complex thereof, exemplary druggable regions including binding pockets and sites, enzymatic active sites, interfaces between domains of a polypeptide or complex, surface grooves or contours or surfaces of a polypeptide or complex which are capable of participating in interactions with another molecule. In some embodiments, the interacting molecule is another polypeptide, which can be naturally occurring. In some embodiments, the druggable region is on the surface of the molecule. In some embodiments, a druggable region is a GAPDHS binding pocket.

Druggable regions can be described and characterized in a number of ways. For example, a druggable region can be characterized by some or all of the amino acids that make up the region, or the backbone atoms thereof, or the side chain atoms thereof (optionally with or without the Ca atoms). Alternatively, In some embodiments, the volume of a druggable region corresponds to that of a carbon based molecule of at least about 200 atomic mass units (amu) and often up to about 800 amu. In some embodiments, it will be appreciated that the volume of such region can correspond to a molecule of at least about 600 amu and often up to about 1600 amu or more.

Alternatively, a druggable region can be characterized by comparison to other regions on the same or other molecules. For example, the term “affinity region” refers to a druggable region on a molecule (such as a polypeptide of the presently disclosed subject matter) that is present in several other molecules, in so much as the structures of the same affinity regions are sufficiently the same so that they are expected to bind the same or related structural analogs. An example of an affinity region is an adenosine triphosphate (ATP)-binding site of a protein kinase that is found in several protein kinases (whether or not of the same origin). Another example of an affinity region is the NAD cofactor-binding site of the various GAPDH isoforms.

The term “selectivity region” refers to a druggable region of a molecule that can not be found on other molecules, in so much as the structures of different selectivity regions are sufficiently different so that they are not expected to bind the same or related structural analogs. An exemplary selectivity region is a catalytic domain of a protein kinase that exhibits specificity for one substrate. In certain instances, a single modulator can bind to the same affinity region across a number of proteins that have a substantially similar biological function, whereas the same modulator can bind to only one selectivity region of one of those proteins.

In this manner, the various isoforms of GAPDH (i.e. GAPDH, GAPDHS, and Gapdhs) comprise a plurality of affinity regions and selectivity regions. As discussed hereinabove, the NAD cofactor-binding pocket acts like an affinity region in that the NAD cofactor-binding pocket from each GAPDH isoform would be expected to bind to NAD. Similarly, the substrate-binding pocket of each isoform would be expected to bind to G3P. However, in the design and provision of modulators that are specific for GAPDHS, the substrate and cofactor-binding pockets are treated as selectivity regions. Put another way, in some embodiments, the design and provision of a modulator that is selective for the male germ cell isoform of GAPDH takes advantage of the amino acid differences between the substrate-binding and cofactor-binding pockets of GAPDH and GAPDHS. These amino acid differences that can be exploited to produce GAPDHS-specific modulators include, but are not limited to, the amino acids listed in Tables 2-5.

Table 2 presents residues conserved in GAPDHS that differ from the conserved residue in the corresponding positions of somatic GAPDH. Conserved residues that are within 20 Å from the substrate-binding site are indicated by bold type.

TABLE 2
Residues Conserved in Spermatogenic GAPDH

| Distance from | | | | | |
|---|---|---|---|---|---|
| Residue C_{α} | | Human | Human | Mouse | Mouse |
| to substrate | Domain* | GAPDH | GAPDHS | Gapdh | Gapdhs |
| 28.6 | NAD | K4 | T77 | K3 | T107 |
| 22.5 | NAD | T18 | L91 | T176 | L121 |
| 23.3 | NAD | A21 | C94 | A20 | C124 |
| 25.3 | NAD | S24 | K97 | S23 | K127 |
| 27.9 | NAD | K26 | — | K25 | — |
| 30.4 | NAD | D28 | K100 | E27 | R130 |
| 31.8 | NAD | L39 | P111 | L38 | P141 |
| 32.4 | NAD | H40 | E112 | N39 | E142 |
| 27.4 | NAD | Q47 | K119 | Q46 | K149 |
| 28.1 | NAD | F55 | Y127 | F54 | Y157 |
| 35.0 | NAD | K60 | E132 | K59 | E162 |
| 38.8 | NAD | E62 | R134 | E61 | K164 |
| 34.1 | NAD | K65 | Q137 | K64 | Q167 |
| 36.1 | NAD | G70 | N142 | G69 | N172 |
| 29.2 | NAD | F76 | Y148 | F75 | Y178 |
| 27.6 | NAD | E78 | C150 | E77 | C180 |
| 31.0 | NAD | K85 | P157 | K84 | P187 |
| 15.4 | NAD | F101 | Y173+ | F100 | Y203 |
| 13.4 | NAD | T102+ | L174 | T101 | L204 |
| 15.6 | NAD | T103 | S175 | T102 | S205+ |
| 18.8 | NAD | M104 | I176 | M103 | I206 |
| 20.4 | NAD | K106 | A178 | K105 | A208 |
| 23.4 | NAD | G108 | S180 | G107 | S210 |
| 28.9 | NAD | K112 | S184 | K111 | S214 |
| 12.4 | NAD | A125 | P197 | A124 | P227 |
| 21.9 | NAD | H136 | E208 | H135 | E238 |
| 21.4 | NAD | K138 | 0210 | K137 | D240 |
| | NAD | — | P213 | — | P243 |
| 26.7 | NAD | N141 | G214 | N140 | G244 |
| 23.8 | NAD | L143 | M216 | L142 | M246 |
| 23.5 | C | D165 | E238 | D164 | E268 |
| 12.2 | C | A179 | S252+ | A178 | S282+ |
| 12.6 | C | I180 | Y253 | I179 | Y283 |
| 16.2 | C | G192 | R265 | G191 | K295 |
| 21.1 | C | A202 | H275 | A201 | H305 |
| 15.0 | C | D224 | K297 | N223 | K327 |
| 27.4 | C | E249 | A322 | E248 | A352 |
| 30.3 | C | D255 | S328 | D254 | S358 |
| 29.6 | C | D256 | A329 | D255 | A359 |
| 28.2 | C | K259 | E332 | K258 | E362 |
| 26.3 | C | V260 | A333 | V259 | A363 |
| 26.6 | C | E263 | A336 | Q262 | A366 |
| 27.4 | C | E266 | K339 | E265 | K369 |
| 23.6 | C | K270 | A343 | K269 | A373 |
| 25.7 | C | G297 | K370 | G296 | K400 |
| 21.0 | C | M327 | L400 | M326 | L430 |
| 23.6 | C | A328 | R401 | A327 | R431 |
| 26.0 | C | A331 | F404 | A330 | F434 |

*NAD: NAD-binding domain; C: catalytic domain
+Predicted by NetPhos 2.0 (Blom et al., 1999) as potential phosphorylation sites

Additionally, using the SYBYL® Site ID program as discussed in Example 6, three major binding pockets and one subpocket were identified. These pockets are referred to herein as Pockets 1-3 and Small Pocket 1, respectively. As used herein, the term “binding pocket” refers to a structural domain of a molecule (e.g. a polypeptide) that is a site for interaction between the molecule and another molecule. With particular reference to enzymes (e.g. GAPDH, GAPDHS, and/or Gapdhs), binding pockets include, but are not limited to substrate-binding domains, cofactor-binding domains, inhibitor binding domains, etc.

Pocket 1 includes the amino acids presented in Table 3 (amino acids that differ between the human somatic isoform (GAPD) and the male germ cell-specific isoforms (GAPDHS/GAPDS) are indicated in bold type):

TABLE 3
Pocket 1 Residues

| Human | Human | Mouse | Rat |
|---|---|---|---|
| GAPD | GAPDHS | GAPDS | GAPDS |
| R12 | R85 | R115 | R109 |
| I13 | I86 | I116 | I110 |
| L16 | L89 | L119 | L113 |
| S121 | S193 | T223 | T217 |
| S150 | S223 | S253 | S247 |
| C151 | C224 | C254 | C248 |
| T152 | T225 | T255 | T249 |
| H178 | H251 | H281 | H275 |
| A179 | S252 | S282 | A276 |
| I180 | Y253 | Y283 | Y277 |
| T181 | T254 | T284 | T278 |
| A182 | A255 | A285 | A279 |
| T210 | T283 | T313 | T307 |
| G211 | G284 | G314 | G308 |
| A237 | P310 | P340 | P334 |
| N315 | N388 | N418 | N412 |
| E316 | E389 | E419 | E413 |
| Y319 | Y392 | Y422 | Y416 |
| S320 | S393 | S423 | S417 |

Additional docking studies were conducted for a smaller subpocket that is more closely confined to the substrate, defined herein as Small Pocket 1. This pocket comprises the following residues: in mouse GAPDS—S253, C254, T255, T313, G314; in human GAPDHS—S223, C224, T225, T283, G284; in rat GAPDS—S247, C248, T249, T307, G308; and in human GAPD—S150, C151, T152, T210, G211.

Pocket 2 includes the amino acids presented in Table 4 (amino acids that differ between the human somatic isoform (GAPD) and the male germ cell-specific isoforms (GAPDS/GAPDHS) are indicated in bold type):

TABLE 4
Pocket 2 Residues

| Human | Human | Mouse | Rat |
|---|---|---|---|
| GAPD | GAPDHS | GAPDS | GAPDS |
| N8 | N81 | N111 | N105 |
| G9 | G82 | G112 | G106 |
| F10 | F83 | F113 | F107 |
| G11 | G84 | G114 | G108 |
| N33 | N105 | N135 | N129 |
| D34 | D106 | D136 | D130 |
| P35 | P107 | P137 | P131 |
| F36 | F108 | F138 | F132 |
| E78 | C150 | C180 | C174 |
| R79 | K151 | K181 | K175 |
| D80 | E152 | D182 | D176 |
| P81 | P153 | P183 | P177 |
| E96 | E168 | E198 | E198 |
| S97 | S169 | C199 | A193 |
| T98 | T170 | T200 | T194 |
| V100 | V172 | V202 | V196 |
| F101 | Y173 | Y203 | Y197 |

As used herein, the term “Pocket 3” refers to a subsequence of a polypeptide that comprises a third pocket that has been identified in the GAPD and GAPDS polypeptides. The term “Pocket 3” refers to the amino acid residues that are in close proximity to the identified pocket. This pocket includes the amino acids presented in Table 5 (amino acids that differ between the human somatic isoform (GAPD) and the male germ cell-specific isoforms (GAPDS/GAPDHS) are indicated in bold type):

TABLE 5
Pocket 3 Residues

| Human | Human | Mouse | RAT |
|---|---|---|---|
| GAPD | GAPDHS | GAPDS | GAPDS |
| T181 | T254 | T284 | T278 |
| A182 | A255 | A285 | A279 |
| T183 | T256 | T286 | T280 |
| Q184 | Q257 | Q287 | Q281 |
| K185 | K258 | K288 | K282 |
| S191 | S264 | S294 | S288 |
| L194 | A267 | D297 | D291 |
| R196 | R269 | R299 | R293 |
| D197 | D270 | G300 | G294 |
| G198 | G271 | G301 | G295 |
| I206 | I279 | I309 | I303 |
| P207 | P280 | P310 | P304 |
| A208 | A281 | S311 | S305 |
| S209 | S282 | S312 | S306 |
| A231 | A304 | A334 | A328 |
| R233 | R306 | R336 | R330 |

Continuing with examples of different druggable regions, the term “undesired region” refers to a druggable region of a molecule that upon interacting with another molecule results in an undesirable effect. For example, a binding site that oxidizes the interacting molecule (such as cytochrome P450 activity) and thereby results in increased toxicity for the oxidized molecule can be deemed an “undesired region”. Other examples of potential undesired regions include regions that upon interaction with a drug decrease the membrane permeability of the drug, increase the excretion of the drug, or increase the blood brain transport of the drug. It can be the case that, in certain circumstances, an undesired region will no longer be deemed an undesired region because the affect of the region will be favorable, i.e., a drug intended to treat a brain condition would benefit from interacting with a region that resulted in increased blood brain transport, whereas the same region could be deemed undesirable for drugs that were not intended to be delivered to the brain.

When used in reference to a druggable region, the “selectivity” or “specificity” of a molecule such as a modulator to a druggable region can be used to describe the binding between the molecule and a druggable region. For example, the selectivity of a modulator with respect to a druggable region can be expressed by comparison to another modulator, using the respective values of K_{d} (i.e., the dissociation constants for each modulator-druggable region complex) or, in cases where a biological effect is observed below the K_{d}, the ratio of the respective EC₅₀'s (i.e., the concentrations that produce 50% of the maximum response for the modulator interacting with each druggable region).

As used herein, the phrase “enhancer-promoter” refers to a composite unit that contains both enhancer and promoter elements. An enhancer-promoter is operatively linked to a coding sequence that encodes at least one gene product.

As used herein, the term “expression” generally refers to the cellular processes by which a polypeptide is produced from RNA.

As used herein, the term “gene” is used for simplicity to refer to a functional protein, polypeptide, or peptide encoding unit. As will be understood by those of ordinary skill in the art, this functional term encompasses both genomic sequences and cDNA sequences. Exemplary embodiments of genomic and cDNA sequences are disclosed herein.

As used herein, the term “GAPDH” refers to nucleic acids encoding a glyceraldehyde 3-phosphate dehydrogenase (GAPDH) polypeptide that can bind one or more ligands. The term “GAPDH” includes invertebrate homologs; however, GAPDH nucleic acids and polypeptides can also be isolated from vertebrate sources. “GAPDH” further includes vertebrate homologs of GAPDH family members, including, but not limited to, mammalian and avian homologs. Representative mammalian homologs of GAPDH family members include, but are not limited to, murine, rat, and human homologs. The term “GAPDH” can also be employed to refer to a polypeptide, which will be apparent to those of ordinary skill in the art upon reflection of the context in which the term is employed herein.

In mammals, there is a male germ cell-specific isoform of GAPDH that is referred to as GAPDHS. Accordingly, the phrase “murine Gapdhs” or mouse Gapdhs” refers to the male germ cell-specific isoform of GAPDH that is found in the mouse. Similarly, the phrase “rat Gapdhs” refers to the male germ cell-specific isoform of GAPDH that is found in the rat. Humans also have a GAPDHS, which is sometimes also referred to as GAPD2 or GAPDH2.

Additionally, consistent with usage in the art, identifications of genes or gene products that are presented in all capital letters refer to human genes and/or gene products or are referring to a family member without reference to the species from which it is derived. For genes and gene products from murine sources (e.g., mice or rats), the first letter is capitalized and other letters are presented in lower case. Also typically, references to genes are presented in italics, and references to polypeptides are presented in normal type. Thus, GAPDHS refers to either a human GAPDHS gene or to a GAPDHS gene generally (i.e., without reference to a particular species). Similarly, GAPDHS refers to a human GAPDHS polypeptide, or to a GAPDHS polypeptide without reference to a particular species of origin. In some embodiments, Gapdhs refers to a mouse or rat Gapdhs gene, and Gapdhs refers to a mouse or rat Gapdhs polypeptide.

The term “GAPDHS”, therefore, in some embodiments refers to a human gene or polypeptide, and not to a specific gene or gene product from a non-human (although recombinant genes or gene products might use the term “GAPDHS” if a portion of the nucleic acid or amino acid sequence is derived from human GAPDHS). Thus, the phrase “GAPDH” can refer to a glyceraldehyde 3-phosphate dehydrogenase generically (i.e. to be inclusive of the somatic and male germ cell-specific isoforms) or, depending on the particular context, can refer specifically to a somatic isoform of glyceraldehyde 3-phosphate dehydrogenase. In the latter case, “GAPDH” would be used to differentiate the somatic isoform (GAPDH) from the male germ cell-specific isoform (“GAPDHS”).

As used herein, the terms “GAPDHS gene” and “recombinant GAPDHS gene” refer to a nucleic acid molecule comprising an open reading frame encoding a GAPDHS polypeptide of the presently disclosed subject matter, including both exon and (optionally) intron sequences.

As used herein, the terms “GAPDHS gene product”, “GAPDHS protein”, “GAPDHS polypeptide”, and “GAPDHS peptide” are used interchangeably and refer to peptides having amino acid sequences which are substantially identical to native amino acid sequences from an organism of interest and which are biologically active in that they comprise all or a part of the amino acid sequence of a GAPDHS polypeptide, or cross-react with antibodies raised against a GAPDHS polypeptide, or retain all or some of the biological activity (e.g., ligand-binding ability) of the native amino acid sequence or protein. Such biological activity can include immunogenicity.

As used herein, the terms “GAPDHS gene product”, “GAPDHS protein”, “GAPDHS polypeptide”, and “GAPDHS peptide” also include analogs of a GAPDHS polypeptide. By “analog” is intended that a DNA or peptide sequence can contain alterations relative to the sequences disclosed herein, yet retain all or some of the biological activity of those sequences. Analogs can be derived from genomic nucleotide sequences as are disclosed herein or from other organisms, or can be created synthetically. Those skilled in the art will appreciate that other analogs, as yet undisclosed or undiscovered, can be used to design and/or construct GAPDHS analogs. There is no need for a “GAPDHS gene product”, “GAPDHS protein”, “GAPDHS polypeptide”, or “GAPDHS peptide” to comprise all or substantially all of the amino acid sequence of a GAPDHS polypeptide gene product. Shorter or longer sequences are anticipated to be of use in the presently disclosed subject matter; shorter sequences are herein referred to as “segments”. Thus, the terms “GAPDHS gene product”, “GAPDHS protein”, “GAPDHS polypeptide”, and “GAPDHS peptide” also include fusion, chimeric or recombinant GAPDHS polypeptides and proteins comprising sequences of the presently disclosed subject matter. Methods of preparing such proteins are disclosed herein and are known in the art.

The term “having substantially similar biological activity”, when used in reference to two polypeptides, refers to a biological activity of a first polypeptide which is substantially similar to at least one of the biological activities of a second polypeptide. A substantially similar biological activity refers to that the polypeptides carry out a similar function, e.g., a similar enzymatic reaction or a similar physiological process, etc. For example, two homologous proteins can have a substantially similar biological activity if they are involved in a similar enzymatic reaction, e.g., they are both kinases which catalyze phosphorylation of a substrate polypeptide, however, they can phosphorylate different regions on the same protein substrate or different substrate proteins altogether. Alternatively, two homologous proteins can also have a substantially similar biological activity if they are both involved in a similar physiological process, e.g., transcription. For example, two proteins can be transcription factors, however, they can bind to different DNA sequences or bind to different polypeptide interactors. Substantially similar biological activities can also be associated with proteins carrying out a similar structural role, for example, two membrane proteins. In some embodiments, GAPDH and GAPDHS have substantially similar biological activities in that in the presence of NAD and phosphate each enzyme catalyzes the conversion of glyceraldehyde 3-phosphate to 1,3-bisphosphoglycerate.

As used herein, the term “hybridization” refers to the binding of a probe molecule, a molecule to which a detectable moiety has been bound, to a target sample. Hybridization can include the pairing of substantially complementary nucleotide sequences (strands of nucleic acid) to form a duplex or heteroduplex by the establishment of hydrogen bonds between complementary base pairs. Hybridization is a specific, i.e. non-random, interaction between two complementary polynucleotides.

As used herein, the term “hyperactivation”, and grammatical derivatives thereof, refers to a change in the motility of sperm from a low amplitude, progressive motility observed in freshly ejaculated or diluted epididymal sperm to a “whiplash”, high amplitude, less progressive motility observed concurrently with capacitation (Yanagimachi, 1994, at pages 189-317). Hyperactivated motility might facilitate sperm transport in the oviducts and is thought to be important for penetration of the zona pellucida surrounding the ovum. See Suarez, 1996.

As used herein, the term “interact” refers to detectable interactions between molecules, such as can be detected using, for example, a yeast two hybrid assay. The term “interact” is also meant to include “binding” interactions and “associations” between molecules. Interactions can, for example, be protein-protein or protein-nucleic acid in nature.

As used herein, the term “isolated” refers to a molecule substantially free of other nucleic acids, proteins, lipids, carbohydrates, and/or other materials with which it is normally associated, such association being either in cellular material or in a synthesis medium. Thus, the term “isolated nucleic acid” refers to a polynucleotide of genomic, cDNA, or synthetic origin or some combination thereof, which (1) is not associated with the cell in which the “isolated nucleic acid” is found in nature, or (2) is operatively linked to a polynucleotide to which it is not linked in nature. Similarly, the term “isolated polypeptide” refers to a polypeptide, in some embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The term “isolated”, when used in the context of an “isolated cell”, refers to a cell that has been removed from its natural environment, for example, as a part of an organ, tissue, or organism. In some embodiments, an isolated cell is a spermatogenic cell that has been isolated (i.e. removed) from the testis. In some embodiments, an isolated cell is a sperm cell that has been isolated (i.e. removed) from the epididymis.

As used herein, the terms “label” and “labeled” refer to the attachment of a moiety, capable of detection by spectroscopic, radiologic, or other methods, to a probe molecule. Thus, the terms “label” or “labeled” refer to incorporation or attachment, optionally covalently or non-covalently, of a detectable marker into a molecule, such as a polypeptide. Various methods of labeling polypeptides are known in the art and can be used. Examples of labels for polypeptides include, but are not limited to, the following: radioisotopes, fluorescent labels, heavy atoms, enzymatic labels or reporter genes, chemiluminescent groups, biotinyl groups, predetermined polypeptide epitopes recognized by a secondary reporter (e.g., leucine zipper pair sequences, binding sites for secondary antibodies, metal binding domains, epitope tags). Examples and use of such labels are described in more detail below. In some embodiments, labels are attached by spacer arms of various lengths to reduce potential steric hindrance.

As used herein, the term “ligand” refers to any compound having the ability to associate with a given target (e.g., a polypeptide). By way of particular example, a polypeptide can be an enzyme (e.g., GAPDHS) and a ligand can be a substrate, a cofactor, or a product (e.g., D-glyceraldehyde-3-phosphate, NAD, or 1,3-bisphosphoglycerate). Thus, the term “ligand” encompasses substrates, cofactors, and products, as well as moieties that can serve as agonists and antagonists; the term also includes moieties that can associate with a site on the polypeptide spatially distant from an active site.

Thus, the term “ligand” refers to any molecule that is known or suspected to associate with another molecule. The term “ligand” encompasses inhibitors, activators, natural substrates, and analogs of natural substrates. In some embodiments, a ligand is a small molecule that binds to a binding pocket of a GAPDH, thereby modulating a biological activity of the GAPDH.

The term “mammal” is known in the art, and exemplary mammals include humans, primates, bovines, porcines, canines, felines, and rodents (e.g., mice and rats).

As used herein, the term “modified” refers to an alteration from an entity's normally occurring state. An entity can be modified by removing discrete chemical units or by adding discrete chemical units. The term “modified” encompasses detectable labels as well as those entities added as aids in purification.

As used herein, the term “modulate” refers to an increase, decrease, or other alteration of any, or all, chemical and biological activities or properties of a biochemical entity, e.g., a wild type or mutant GAPDHS polypeptide. The term “modulation” as used herein refers to both upregulation (i.e., activation or stimulation) and downregulation (i.e., inhibition or suppression) of a response. Thus, the term “modulation”, when used in reference to a functional property or biological activity or process (e.g., enzyme activity or receptor binding), refers to the capacity to upregulate (e.g., activate or stimulate), downregulate (e.g., inhibit or suppress), or otherwise change a quality of such property, activity, or process. In certain instances, such regulation can be contingent on the occurrence of a specific event, such as activation of a signal transduction pathway, and/or can be manifest only in particular cell types.

The term “modulator” refers to a polypeptide, nucleic acid, macromolecule, complex, molecule, small molecule, compound, species, or the like (naturally occurring or non-naturally occurring), or an extract made from biological materials such as bacteria, plants, fungi, or animal cells or tissues, that can be capable of causing modulation. Modulators can be evaluated for potential activity as inhibitors or activators (directly or indirectly) of a functional property, biological activity or process, or combination of them, (e.g., agonist, partial antagonist, partial agonist, inverse agonist, antagonist, anti-microbial agents, inhibitors of microbial infection or proliferation, and the like) by inclusion in assays. In such assays, many modulators can be screened at one time. The activity of a modulator can be known, unknown, or partially known.

Modulators can be either selective or non-selective. As used herein, the term “selective” when used in the context of a modulator (e.g. an inhibitor) refers to a measurable or otherwise biologically relevant difference in the way the modulator interacts with one molecule (e.g. GAPDHS) versus another similar but not identical molecule (e.g. GAPDH). As such, a “selective modulator of GAPDHS” is intended to refer to a modulator that interacts with GAPDHS in a way that is qualitatively different than the way the same modulator would interact with a somatic isoform of GAPDH.

It must be understood that it is not required that the degree to which the interactions differ be completely opposite. Put another way, the term selective modulator encompasses not only those molecules that only bind one or the other of GAPDHS and somatic GAPDH. The term is also intended to include modulators that are characterized by interactions with GAPDHS and somatic GAPDH that differ to a lesser degree. For example, selective modulators include modulators for which conditions can be found (such as local concentrations of the modulator) that would allow a biologically relevant difference in the binding of the modulator to GAPDHS versus GAPDH. Selective modulators also include the modulators listed in Tables 7, 9, and 11, for which the binding of the modulator to GAPDHS and the binding of the modulator to GAPDH are predicted to be different (as shown in the different “Binding Scores”).

When a selective modulator is identified, the modulator will bind to one molecule (for example GAPDHS) in a manner that is different (for example, stronger) than it binds to another molecule (for example, the somatic isoform of GAPDH). As used herein, the modulator is said to display “selective binding” or “preferential binding” to the molecule to which it binds more strongly.

As used herein, the term “molecular replacement” refers to a method of solving the three-dimensional structure of a compound (e.g., a protein) that involves generating a preliminary model of a crystal (e.g., a crystal of a native GAPDH or of a mutant GAPDH) whose structure coordinates are unknown, by orienting and positioning a molecule whose structure coordinates are known within the unit cell of the unknown crystal so as best to account for the observed diffraction pattern of the unknown crystal. Molecular replacement operations can be conveniently carried out on a computer running a suitable software package, such as AmoRe (Navaza & Saludjian, 1997). Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. See e.g., Lattman, 1985; Rossmann, 1972.

The term “motif” refers to an amino acid sequence that is commonly found in a protein of a particular structure or function. Typically, a consensus sequence is defined to represent a particular motif. The consensus sequence need not be strictly defined and can contain positions of variability, degeneracy, variability of length, etc. The consensus sequence can be used to search a database to identify other proteins that can have a similar structure or function due to the presence of the motif in its amino acid sequence. For example, on-line databases can be searched with a consensus sequence in order to identify other proteins containing a particular motif. Various search algorithms and/or programs can be used, including FASTA, BLAST, or ENTREZ. FASTA and BLAST are available as a part of the GCG® WISCONSIN PACKAGE® (Accelrys, Inc., San Diego, Calif., United States of America). ENTREZ is available through the National Center for Biotechnology Information, National Library of Medicine, National Institutes of Health, Bethesda, Md., United States of America.

As used herein, the term “mutation” carries its traditional connotation and refers to a change, inherited, naturally occurring, or introduced, in a nucleic acid or polypeptide sequence, and is used in its sense as generally known to those of skill in the art.

As used herein, the term “NAD cofactor-binding domain” refers to a subsequence of a polypeptide that comprises the NAD cofactor-binding pocket. Thus, the term “NAD cofactor-binding domain” is not intended herein to refer specifically to those amino acids that interact specifically with NAD. Rather, the term is used very broadly to include interacting residues as well as surrounding residues. The term “NAD cofactor-binding pocket” refers more particularly to the amino acid residues that are in close proximity to the site at which the NAD cofactor binds to a GAPDH polypeptide.

The term “naturally occurring”, as applied to an object, refers to the fact that an object can be found in nature. For example, a polypeptide or polynucleotide sequence that is present in an organism (including bacteria) that can be isolated from a source in nature and which has not been intentionally modified by man in the laboratory is naturally occurring.

As used herein, the terms “nucleic acid” and “nucleic acid molecule” refer to any of deoxyribonucleic acid (DNA), ribonucleic acid (RNA), oligonucleotides, fragments generated by the polymerase chain reaction (PCR), and fragments generated by any of ligation, scission, endonuclease action, and exonuclease action. Nucleic acids can be composed of monomers that are naturally occurring nucleotides (such as deoxyribonucleotides and ribonucleotides), or analogs of naturally occurring nucleotides (e.g., α-enantiomeric forms of naturally occurring nucleotides), or a combination of both. Modified nucleotides can have modifications in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid” also includes so-called “peptide nucleic acids”, which comprise naturally occurring or modified nucleic acid bases attached to a polyamide backbone. Nucleic acids can be either single stranded or double stranded.

The term “operatively linked”, when describing the relationship between two nucleic acid regions, refers to a juxtaposition wherein the regions are in a relationship permitting them to function in their intended manner. For example, a control sequence “operatively linked” to a coding sequence is ligated in such a way that expression of the coding sequence is achieved under conditions compatible with the control sequences, such as when the appropriate molecules (e.g., inducers and polymerases) are bound to the control or regulatory sequence(s). Thus, in some embodiments, the phrase “operatively linked” refers to that an enhancer-promoter is connected to a coding sequence in such a way that the transcription of that coding sequence is controlled and regulated by that enhancer-promoter. Techniques for operatively linking an enhancer-promoter to a coding sequence are well known in the art; the precise orientation and location relative to a coding sequence of interest is dependent, inter alia, upon the specific nature of the enhancer-promoter.

The phrases “percent identity” and “percent identical,” in the context of two nucleic acid or protein sequences, refer to two or more sequences or subsequences that have in some embodiments at least 60%, in some embodiments at least 70%, in some embodiments at least 80%, in some embodiments at least 85%, in some embodiments at least 90%, in some embodiments at least 95%, in some embodiments at least 98%, and in some embodiments at least 99% nucleotide or amino acid residue identity, when compared and aligned for maximum correspondence, as measured using one of the following sequence comparison algorithms or by visual inspection. The percent identity exists in some embodiments over a region of the sequences that is at least about 50 residues in length, in some embodiments over a region of at least about 100 residues, and in some embodiments the percent identity exists over at least about 150 residues. In some embodiments, the percent identity exists over the entire length of a given region, such as a coding region.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm described in Smith & Waterman 1981, by the homology alignment algorithm described in Needleman & Wunsch 1970, by the search for similarity method described in Pearson & Lipman 1988, by computerized implementations of these algorithms (GAP, BESTFIT, FASTA, and TFASTA in the GCG Wisconsin Package, available from Accelrys, Inc., San Diego, Calif., United States of America), or by visual inspection. See generally, Ausubel et al., 1989.

One example of an algorithm that is suitable for determining percent sequence identity and sequence similarity is the BLAST algorithm, which is described in Altschul et al., 1990. Software for performing BLAST analyses is publicly available through the website of the National Center for Biotechnology Information (NCBI). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff 1989.

In addition to calculating percent sequence identity, the BLAST algorithm also performs a statistical analysis of the similarity between two sequences. See e.g., Karlin & Altschul 1993. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

The term “substantially identical”, in the context of two nucleotide or amino acid sequences, refers to two or more sequences or subsequences that have in some embodiments at least about 80% nucleotide or amino acid identity, in some embodiments at least about 85% nucleotide or amino acid identity, in some embodiments at least about 90% nucleotide or amino acid identity, in some embodiments at least about 95% nucleotide or amino acid identity, in some embodiments at least about 98% nucleotide or amino acid identity, and in some embodiments at least about 99% nucleotide or amino acid identity, when compared and aligned for maximum correspondence, as measured using one of the following sequence comparison algorithms or by visual inspection. With regard to nucleic acid sequences, the substantial identity exists in some embodiments in nucleotide sequences of at least 50 residues, in some embodiments in nucleotide sequence of at least about 100 residues, in some embodiments in nucleotide sequences of at least about 150 residues, and in some embodiments in nucleotide sequences comprising complete coding sequences. In one aspect, polymorphic sequences can be substantially identical sequences. The term “polymorphic” refers to the occurrence of two or more genetically determined alternative sequences or alleles in a population. An allelic difference can be as small as one base pair. Nonetheless, one of ordinary skill in the art would recognize that the polymorphic sequences correspond to the same gene.

With regard to amino acid sequences, the substantial identity exists in some embodiments in amino acid sequences of at least about 10 amino acids, in some embodiments in amino acid sequences of at least about 20 residues, in some embodiments in amino acid sequences of at least about 30 residues, in some embodiments in amino acid sequences of at least about 40 residues, in some embodiments in amino acid sequences of at least about 50 residues, in some embodiments in amino acid sequences of at least about 100 residues, and in some embodiments in amino acid sequences comprising complete amino acid sequences of a polypeptide. In certain instances residue positions that are not identical differ by conservative amino acid substitutions, which are described herein.

Another indication that two nucleotide sequences are substantially identical is that the two molecules specifically or substantially hybridize to each other under stringent conditions. In the context of nucleic acid hybridization, two nucleic acid sequences being compared can be designated a “probe sequence” and a “target sequence”. A “probe sequence” is a reference nucleic acid molecule, and a ““target sequence” is a test nucleic acid molecule, often found within a heterogeneous population of nucleic acid molecules. A “target sequence” is synonymous with a “test sequence”.

An exemplary nucleotide sequence employed for hybridization studies or assays includes probe sequences that are complementary to or mimic in some embodiments at least an about 14 to 40 nucleotide sequence of a nucleic acid molecule of the presently disclosed subject matter. In one example, probes comprise 14 to 20 nucleotides, or even longer where desired, such as 30, 40, 50, 60, 100, 200, 300, or 500 nucleotides or up to the full length of a given gene. Such fragments can be readily prepared by, for example, directly synthesizing the fragment by chemical synthesis, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

The term uphenotype” refers to the entire physical, biochemical, and physiological makeup of a cell or an organism, e.g., having any one trait or any group of traits.

As used herein, the terms “polypeptide”, “protein”, and “peptide”, which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product. The term “polypeptide” encompasses proteins of all functions, including enzymes. Thus, exemplary polypeptides include gene products, naturally occurring proteins, homologs, orthologs, paralogs, fragments, and other equivalents, variants and analogs of the foregoing.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least 5, 6, 8, or 10 amino acids long, at least 14 amino acids long, at least 20, 30, 40, or 50 amino acids long, at least 75 amino acids long, or at least 100, 150, 200, 300, 500, or more amino acids long. A fragment can retain one or more of the biological activities of the reference polypeptide. In some embodiments, a fragment can comprise a druggable region, and optionally additional amino acids on one or both sides of the druggable region, which additional amino acids can number from 5, 10, 15, 20, 30, 40, 50, or up to 100 or more residues. Further, fragments can include a sub-fragment of a specific region, which sub-fragment retains a function of the region from which it is derived. In some embodiments, a fragment can have immunogenic properties.

As used herein, the term “primer” refers to a sequence comprising in some embodiments two or more deoxyribonucleotides or ribonucleotides, in some embodiments more than three, in some embodiments more than eight, and in some embodiments at least about 20 nucleotides of an exonic or intronic region. Such oligonucleotides are in some embodiments between ten and thirty bases in length.

The term “purified” refers to an object species that is the predominant species present (i.e., on a molar basis it is more abundant than any other individual species in the composition). A “purified fraction” is a composition wherein the object species comprises at least about 50 percent (on a molar basis) of all species present. In making the determination of the purity of a species in solution or dispersion, the solvent or matrix in which the species is dissolved or dispersed is usually not included in such determination; instead, only the species (including the one of interest) dissolved or dispersed are taken into account. Generally, a purified composition will have one species that comprises more than about 80 percent of all species present in the composition, more than about 85%, 90%, 95%, 99%, or more of all species present. The object species can be purified to essential homogeneity (contaminant species cannot be detected in the composition by conventional detection methods) wherein the composition consists essentially of a single species. A skilled artisan can purify a polypeptide of the presently disclosed subject matter using standard techniques for protein purification in light of the teachings herein. Purity of a polypeptide can be determined by a number of methods known to those of skill in the art, including for example, amino-terminal amino acid sequence analysis, gel electrophoresis, and mass-spectrometry analysis.

The terms “recombinant protein” or “recombinant polypeptide” refer to a polypeptide that is produced by recombinant DNA techniques. An example of such techniques includes the case when DNA encoding the expressed protein is inserted into a suitable expression vector that is in turn used to transform a host cell to produce the protein or polypeptide encoded by the DNA.

A “reference sequence” is a defined sequence used as a basis for a sequence comparison. A reference sequence can be a subset of a larger sequence, for example, as a segment of a full-length nucleotide or amino acid sequence, or can comprise a complete sequence. Generally, when used to refer to a nucleotide sequence, a reference sequence is at least 200, 300 or 400 nucleotides in length, frequently at least 600 nucleotides in length, and often at least 800 nucleotides in length. Because two proteins can each (1) comprise a sequence (i.e., a portion of the complete protein sequence) that is similar between the two proteins, and (2) can further comprise a sequence that is divergent between the two proteins, sequence comparisons between two (or more) proteins are typically performed by comparing sequences of the two proteins over a “comparison window” (defined hereinabove) to identify and compare local regions of sequence similarity.

The term “regulatory sequence” is a generic term used throughout the specification to refer to polynucleotide sequences, such as initiation signals, enhancers, regulators and promoters, which are necessary or desirable to affect the expression of coding and non-coding sequences to which they are operatively linked. Exemplary regulatory sequences are described in Goeddel, 1990, and include, for example, the early and late promoters of simian virus 40 (SV40), adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. The nature and use of such control sequences can differ depending upon the host organism. In prokaryotes, such regulatory sequences generally include promoter, ribosomal binding site, and transcription termination sequences. The term “regulatory sequence” is intended to include, at a minimum, components whose presence can influence expression, and can also include additional components whose presence is advantageous, for example, leader sequences and fusion partner sequences. In some embodiments, transcription of a polynucleotide sequence is under the control of a promoter sequence (or other regulatory sequence) that controls the expression of the polynucleotide in a cell-type in which expression is intended. It will also be understood that the polynucleotide can be under the control of regulatory sequences that are the same or different from those sequences which control expression of the naturally occurring form of the polynucleotide.

The term “reporter gene” refers to a nucleic acid comprising a nucleotide sequence encoding a protein that is readily detectable either by its presence or activity, including, but not limited to, luciferase, fluorescent protein (e.g., green fluorescent protein), chloramphenicol acetyl transferase, β-galactosidase, secreted placental alkaline phosphatase, β-lactamase, human growth hormone, and other secreted enzyme reporters. Generally, a reporter gene encodes a polypeptide not otherwise produced by the host cell, which is detectable by analysis of the cell(s), e.g., by the direct fluorometric, radioisotopic or spectrophotometric analysis of the cell(s) and typically without the need to kill the cells for signal analysis. In certain instances, a reporter gene encodes an enzyme, which produces a change in fluorometric properties of the host cell, which is detectable by qualitative, quantitative, or semiquantitative function or transcriptional activation. Exemplary enzymes include esterases, β-lactamase, phosphatases, peroxidases, proteases (tissue plasminogen activator or urokinase) and other enzymes whose function can be detected by appropriate chromogenic or fluorogenic substrates known to those skilled in the art or developed in the future.

As used herein, the term “sequencing” refers to determining the ordered linear sequence of nucleic acids or amino acids of a DNA or protein target sample, using conventional manual or automated laboratory techniques.

The term “small molecule” refers to a compound, which has a molecular mass of in some embodiments less than about 5 kilodaltons (kDa), in some embodiments less than about 2.5 kDa, in some embodiments less than about 1.5 kDa, and in some embodiments less than about 0.9 kDa. Small molecules can be, for example, nucleic acids, peptides, polypeptides, peptide nucleic acids, peptidomimetics, carbohydrates, lipids, or other organic (carbon containing) or inorganic molecules. Many pharmaceutical companies have extensive libraries of chemical and/or biological mixtures, often fungal, bacterial, or algal extracts, which can be screened with any of the assays of the presently disclosed subject matter. The term “small organic molecule” refers to a small molecule that is often identified as being an organic or medicinal compound, and does not include molecules that are exclusively nucleic acids, peptides, or polypeptides. In some embodiments, a small organic compound has a molecular mass of between about 0.05 kDa and 2.5 kDa.

The term “soluble” as used herein with reference to a polypeptide of the presently disclosed subject matter or other protein, refers to that upon expression in cell culture, at least some portion of the polypeptide or protein expressed remains in the cytoplasmic fraction of the cell and does not fractionate with the cellular debris upon lysis and centrifugation of the lysate. Solubility of a polypeptide can be increased by a variety of art recognized methods, including fusion to a heterologous amino acid sequence, deletion of amino acid residues, amino acid substitution (e.g., enriching the sequence with amino acid residues having hydrophilic side chains), and chemical modification (e.g., addition of hydrophilic groups). The solubility of polypeptides can be measured using a variety of art recognized techniques, including, dynamic light scattering to determine aggregation state, UV absorption, centrifugation to separate aggregated from non-aggregated material, and sodium dodecyl sulfate (SDS) gel electrophoresis (e.g., the amount of protein in the soluble fraction is compared to the amount of protein in the soluble and insoluble fractions combined). When expressed in a host cell, the polypeptides of the presently disclosed subject matter can be at least about 1%, 2%, 5%, 10%, 20%, 30%, 40%, 50%, 60%, 70%, 80%, 90%, or more soluble, e.g., at least about 1%, 2%, 5%, 10%, 20%, 30%, 40%, 50%, 60%, 70%, 80%, 90%, or more of the total amount of protein expressed in the cell is found in the cytoplasmic fraction. In some embodiments, a one liter culture of cells expressing a polypeptide of the presently disclosed subject matter will produce at least about 0.1, 0.2, 0.5, 1, 2, 5, 10, 20, 30, 40, 50 milligrams, or more of soluble protein. In some embodiments, a polypeptide of the presently disclosed subject matter is at least about 10% soluble and will produce at least about 1 milligram of protein from a one liter cell culture.

As used herein, the term “somatic isoform” refers to an isoform of a polypeptide (e.g. a GAPDH) that is found in somatic cells. Certain polypeptides have multiple isoforms, and in many cases a germ cell-specific isoform exists. Usually, this germ cell-specific isoform is present in male germ cells, particularly in cells that are undergoing spermatogenesis. For these polypeptides, the somatic isoform of the polypeptide and the germ cell-specific isoform can be encoded by the same gene (with alternative splicing and/or the use of an alternative transcription start site generating the different isoforms) or by different genes. Accordingly, the amino acid sequence of a “somatic isoform” of a polypeptide is different than that of a “germ cell-specific isoform”, the latter of which is also referred to herein as a “male germ cell-specific isoform”, a “male germ cell-specific isoform”, or a “spermatogenic cell isoform”.

The term “specifically hybridizes” refers to detectable and specific nucleic acid binding. Polynucleotides, oligonucleotides, and nucleic acids of the presently disclosed subject matter selectively hybridize to nucleic acid strands under hybridization and wash conditions that minimize appreciable amounts of detectable binding to nonspecific nucleic acids. Stringent conditions can be used to achieve selective hybridization conditions as known in the art and discussed herein. Generally, the nucleic acid sequence homology between the polynucleotides, oligonucleotides, and nucleic acids of the presently disclosed subject matter and a nucleic acid sequence of interest will be at least 30%, 40%, 50%, 60%, 70%, 80%, 85%, 90%, 95%, 98%, 99%, or more. In certain instances, hybridization and washing conditions are performed under stringent conditions according to conventional hybridization procedures and as described further herein.

The terms “stringent conditions” or “stringent hybridization conditions” refer to conditions under which a test nucleic acid molecule will hybridize to a target reference nucleotide sequence, to a detectably greater degree than other sequences (e.g., at least two-fold over background). Stringent conditions are sequence-dependent and will differ in experimental contexts. For example, longer sequences hybridize specifically at higher temperatures. In some embodiments, stringent conditions are selected to be about 5° C. to about 20° C. lower, and in some embodiments 5° C. lower, than the thermal melting point (Tₘ) for the specific target sequence at a defined ionic strength and pH. The Tₘ is the temperature (under defined ionic strength and pH) at which 50% of a complementary target sequence hybridizes to a perfectly matched probe. Typically, stringent conditions are those in which the salt concentration is less than about 1.0 M sodium ion concentration (or other salts), typically about 0.01 to 1.0 M Na ion concentration (or other salts), at pH 7.0 to 8.3, and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide. Exemplary low stringency conditions include hybridization with a buffer solution of 30% formamide, 1 M NaCl, 1% SDS at 37° C., and a wash in 2× standard saline citrate (SSC; 2×SSC is 0.03 M sodium chloride; 0.003 M sodium citrate, pH 7.0) at 50° C. Exemplary high stringency conditions include 6×SSC, 0.2% polyvinylpyrrolidone, 0.2% Ficoll, 0.2% bovine serum albumin, 0.1% sodium dodecyl sulfate, 100 mg/ml salmon sperm DNA and 15% formamide at 60° C.

A variety of techniques for estimating the Tₘ are available. Typically, G-C base pairs in a duplex are estimated to contribute about 3° C. to the Tₘ, while A-T base pairs are estimated to contribute about 2° C., up to a theoretical maximum of about 80-100° C. However, more sophisticated models of Tₘ are available in which G-C stacking interactions, solvent effects, the desired assay temperature, and the like are taken into account. For example, probes can be designed to have a dissociation temperature (Td) of approximately 60° C., using the formula: T_{d}=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the formation of the duplex.

Hybridization can be carried out, for example, in 5×SSC, 4×SSC, 3×SSC, 2×SSC, 1×SSC, or 0.2×SSC for at least about 1 hour, 2 hours, 5 hours, 12 hours, or 24 hours. The temperature of the hybridization can be increased to adjust the stringency of the reaction, for example, from about 25° C. (room temperature), to about 45° C., 50° C., 55° C., 60° C., or 65° C. The hybridization reaction can also include another agent affecting the stringency, for example, hybridization conducted in the presence of 50% formamide increases the stringency of hybridization at a defined temperature.

The hybridization reaction can be followed by a single wash step, or two or more wash steps, which can be at the same or a different salinity and temperature. For example, the temperature of the wash can be increased to adjust the stringency from about 25° C. (room temperature), to about 45° C., 50° C., 55° C., 60° C., 65° C., or higher. The wash step can be conducted in the presence of a detergent, e.g., 0.1, 0.2%, 0.5%, or 1.0% SDS. For example, hybridization can be followed by two wash steps at 65° C. each for about 20 minutes in 2×SSC, 0.1% SDS, and optionally two additional wash steps at 65° C. each for about 20 minutes in 0.2×SSC, 0.1% SDS.

Exemplary stringent hybridization conditions include overnight hybridization at 65° C. in a solution comprising, or consisting of, 50% formamide, 10× Denhardt's (0.2% Ficoll, 0.2% polyvinylpyrrolidone, 0.2% bovine serum albumin) and 200 μg/ml of denatured carrier DNA, e.g., sheared salmon sperm DNA, followed by two wash steps at 65° C. each for about 20 minutes in 2×SSC, 0.1% SDS, and two wash steps at 65° C. each for about 20 minutes in 0.2×SSC, 0.1% SDS.

Hybridization can consist of hybridizing two nucleic acids in solution, or a nucleic acid in solution to a nucleic acid attached to a solid support, e.g., a filter. When one nucleic acid is on a solid support, a prehybridization step can be conducted prior to hybridization. Prehybridization can be carried out for at least about 1 hour, 3 hours, or 10 hours in the same solution and at the same temperature as the hybridization solution (without the complementary polynucleotide strand).

Appropriate stringency conditions are known to those skilled in the art or can be determined experimentally by the skilled artisan. See e.g., Ausubel et al., 1989; Sambrook & Russell, 2001; Agrawal, 1993; Tijssen, 1993; Tibanyenda et al., 1984; and Ebel et al., 1992.

The term “structural motif”, when used in reference to a polypeptide, refers to a polypeptide that, although it can have different amino acid sequences, can result in a similar structure, wherein by structure is meant that the motif forms generally the same tertiary structure, or that certain amino acid residues within the motif, or alternatively their backbone or side chains (which can or can not include the Cα atoms of the side chains) are positioned in a like relationship with respect to one another in the motif.

As used herein, the term “substantially pure” refers to that the polynucleotide or polypeptide is substantially free of the sequences and molecules with which it is associated in its natural state, and those molecules used in the isolation procedure. The term “substantially free” refers to that the sample is in some embodiments at least 50%, in some embodiments at least 70%, in some embodiments 80%, and in some embodiments 90% free of the materials and compounds with which is it associated in nature.

As used herein, the terms “substrate-binding domain” and “catalytic domain” are used interchangeably and refer to a subsequence of a polypeptide that comprises the substrate-binding pocket. Thus, the term “substrate-binding domain” is not intended herein to refer specifically to those amino acids that interact specifically with a substrate. Rather, the term is used very broadly to include interacting residues as well as surrounding residues. The term “substrate-binding pocket” refers more particularly to the amino acid residues that are in close proximity to the site at which the substrate binds to a GAPDH polypeptide.

As used herein, the term “target cell” refers to a cell, into which it is desired to insert a nucleic acid sequence or polypeptide, or to otherwise effect a modification from conditions known to be standard in the unmodified cell. A nucleic acid sequence introduced into a target cell can be of variable length. Additionally, a nucleic acid sequence can enter a target cell as a component of a plasmid or other vector or as a naked sequence.

As used herein, the term “therapeutic agent” is a chemical entity intended to effectuate a change in an organism. In one example, the organism is a human being. It is not necessary that a therapeutic agent be known to effectuate a change in an organism; chemical entities that are suspected, predicted, or designed to effectuate a change in an organism are therefore encompassed by the term “therapeutic agent”. The effectuated change can be of any kind, observable or unobservable, and can include, for example, a change in the biological activity of a protein.

Representative therapeutic compounds include small molecules, proteins and peptides, oligonucleotides of any length, “xenobiotics”, such as drugs and other therapeutic agents, carcinogens and environmental pollutants, natural products and extracts, as well as “endobiotics”, such as epoxycholesterols. Other examples of therapeutic agents can include, but are not restricted to, agonists and antagonists of an enzyme (e.g., a GAPDHS polypeptide), toxins and venoms, viral epitopes, hormones (e.g., opioid peptides, steroids, etc.), hormone receptors, enzymes, enzyme substrates, cofactors, lectins, sugars, nucleic acids, oligosaccharides, and monoclonal antibodies.

The term “therapeutically effective amount” refers to that amount of a modulator, drug, or other molecule that is sufficient to effect treatment when administered to a subject in need of such treatment. The therapeutically effective amount will vary depending upon the subject and condition being treated, the weight and age of the subject, the nature of the condition, the manner of administration and the like, which can readily be determined by one of ordinary skill in the art.

As used herein, the term “transcription” refers to a cellular process involving the interaction of an RNA polymerase with a gene that directs the expression as RNA of the structural information present in the coding sequences of the gene. The process includes, but is not limited to, the following steps: (a) the transcription initiation; (b) transcript elongation; (c) transcript splicing; (d) transcript capping; (e) transcript termination; (f) transcript polyadenylation; (g) nuclear export of the transcript; (h) transcript editing; and (i) stabilizing the transcript.

As used herein, the term “transcription factor” refers to a cytoplasmic or nuclear protein which binds to a gene, or binds to an RNA transcript of a gene, or binds to another protein which binds to a gene or an RNA transcript or another protein which in turn binds to a gene or an RNA transcript, so as to thereby modulate expression of the gene. Such modulation can additionally be achieved by other mechanisms; the essence of a “transcription factor for a gene” pertains to a factor that alters the level of transcription of the gene in some way.

The term “transfection” refers to the introduction of a nucleic acid, e.g., an expression vector, into a recipient cell, which in certain instances involves nucleic acid-mediated gene transfer. The term “transformation” refers to a process in which a cell's genotype is changed as a result of the cellular uptake of exogenous nucleic acid. For example, a transformed cell can express a recombinant form of a polypeptide of the presently disclosed subject matter or antisense expression can occur from the transferred gene so that the expression of a naturally occurring form of the gene is disrupted.

The term “vector” refers to a nucleic acid capable of transporting another nucleic acid to which it has been linked. One type of vector that can be used in accord with the presently disclosed subject matter is an episome, i.e., a nucleic acid capable of extra-chromosomal replication. Other vectors include those capable of autonomous replication and expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA molecules that, in their vector form are not bound to the chromosome. In the present specification, “plasmid” and “vector” are used interchangeably as the plasmid is the most commonly used form of vector. However, the presently disclosed subject matter is intended to include such other forms of expression vectors which serve equivalent functions and which become known in the art subsequently hereto.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

III. Treatment Methods

Disclosed herein is a method of modulating reproduction (e.g., by providing contraception) comprising administering an effective amount of a GAPDHS activity inhibitor to a subject in need thereof.

Also disclosed herein is a method of modulating sperm motility in a subject in which said modulation is desired, the method comprising administering an effective amount of a GAPDHS activity modulator to the subject.

Also disclosed herein is a method for modulating GAPDHS activity in a subject, the method comprising (a) preparing a composition comprising a modulator identified according to a method disclosed herein, and a pharmaceutically acceptable carrier; and (b) administering an effective dose of the composition to a subject.

The term “sperm motility” can include any characteristic of locomotion displayed by sperm cells, such as mammalian sperm cells. For example, this term includes, but is not limited to progressive motility, hyperactivated motility, and combinations thereof.

The term “subject” as used herein refers to any invertebrate or vertebrate species. The methods of the presently disclosed subject matter are particularly useful in the treatment of warm-blooded vertebrates, for instance, mammals and birds. In some embodiments, the animal can be selected from the group consisting of rodent, swine, bird, ruminant, and primate. In some embodiments, the animal can be selected from the group consisting of a mouse, a rat, a pig, a guinea pig, poultry, an emu, an ostrich, a goat, a cow, a sheep, and a rabbit. In some embodiments, the animal can be a primate, such as an ape, a monkey, a lemur, a tarsier, a marmoset, or a human.

Thus, provided is the treatment of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, e.g., poultry, such as turkeys, chickens, ducks, geese, guinea fowl, and the like, as they are also of economical importance to humans. Thus, provided is the treatment of livestock, including, but not limited to, domesticated swine (pigs and hogs), ruminants, horses, poultry, and the like.

Another use of an inhibitor is for pest control. An inhibitor administered to a pest population, such as rats, would inhibit their reproduction and reduce their numbers.

III.A. GAPDHS Modulators

GAPDHS modulators are used in the present methods for modulating GAPDHS activity in cells and tissues. Thus, as used herein, the terms “modulate”, “modulating”, and “modulator” are meant to be construed to encompass inhibiting, blocking, promoting, stimulating, agonizing, antagonizing, or otherwise affecting GAPDHS activity in cells and tissues. The term “effect”, as used herein such in the phrase “having an effect on a biological activity”, is meant to be synonymous with the term “modulate”.

In some embodiments, a GAPDHS activity inhibiting composition is employed in accordance with the presently disclosed subject matter. The terms “composition exhibiting GAPDHS inhibition activity”, “GAPDHS inhibitor” or “GAPDHS inhibiting composition” are used interchangeably and are meant to refer to a substance that acts by inhibiting, blocking, antagonizing, down-regulating, or otherwise reducing GAPDHS activity in cells and tissues. In some embodiments, the inhibitor is selective (as defined herein) for GAPDHS inhibition as compared to GAPDH inhibition.

Summarily, “selective” can refer to the characteristic that at a given set of reactions conditions (for example, physiological temperature and a set dosage of inhibitor), the inhibitor will inhibit GAPDHS to a greater degree than it does GAPDH. In this context, the terms “selectively” and “preferentially” are synonymous and are used interchangeably herein. Thus, the term “preferentially binds”, as used herein, refers to a binding of a molecule (e.g., a GAPDHS inhibitor) that under a given set of conditions occurs more readily to one molecule (e.g., a GAPDHS polypeptide) than to another molecule (e.g., a GAPDH polypeptide).

Other inhibitors or antagonists can be antibodies, peptides, proteins, nucleic acids, small organic molecules, or polymers. In some embodiments the antagonist is a small organic molecule. In some embodiments the antagonist is an antibody. The antibody can be a monoclonal or polyclonal antibody. The antibody can be chemically linked to another organic or bio-molecule. Monoclonal and polyclonal antibodies can be made by any method generally known to those of ordinary skill in the art. For example, U.S. Pat. No. 5,422,245 to Nielsen et al. (assignee: Fonden Til Fremme AF Eksperimental Cancerforskning of Copenhagen, Denmark) describes the production of monoclonal antibodies to plasminogen activator inhibitor.

Peptides, proteins, nucleic acids, small organic molecules, and polymers can be identified by combinatorial methods.

In any of the foregoing embodiments, the GAPDHS can be human GAPDHS. In this case, the GAPDHS modulator can interact with one or more residues in human GAPDHS including, but not limited to N81, G82, F83, G84, R85, I86, G87, L89, N105, D106, P107, F108, C150, K151, E152, P153, E168, S169, T170, V172, Y173, L174, S175, A178, S193 P197, S223, C224, T225, H251, S252, Y253, T254, A255, T256, Q257, K258, S264, R265, A267, R269, D270, G271, I279, P280, A281, S282, T283, G284, A304, R306, P310, N388, E389, Y392, and S393 of SEQ ID NO: 6.

Additionally, the GAPDHS can be mouse Gapdhs. In this case, the Gapdhs modulator can interact with one or more residues in mouse Gapdhs including, but not limited to N111, G112, F113, G114, R115, I116, L119, N135, D136, P137, F138, C180, K181, D182, P183, E198, C199, T200, V202, Y203, L204, S205, A208, T223, P227, S253, C254, T255, H281, S282, Y283, T284, A285, T286, Q287, K288, S294, K295, D297, R299, G300, G301, I309, P310, S311, S312, T313, G314, A334, R336, P340, N418, E419, Y422, and S423 of SEQ ID NO: 2.

Additionally, the GAPDHS can be rat GAPDHS. In this case, the GAPDHS modulator can interact with one or more residues including, but not limited to N105, G106, F107, G108, R109, I110, L113, N129, D130, P131, F132, C174, K175, E176, P177, E192, A193, T194, V196, Y197, L198, S199, A202, T217, P221, S247, C248, T249, H275, A276, Y277, T278, A279, T280, Q281, K282, S288, K289, D291, R293, G294, G295, 1303, P304, S305, S306, T307, G308, A328, R330, P334, N412, E413, Y416, and S417 of SEQ ID NO: 4.

The interaction can be selected from the group consisting of a van der Waals interaction, a hydrophobic interaction, hydrogen bonding, and combinations thereof.

A GAPDHS inhibitor or antagonist can be administered at an effective dose or concentration. Representative concentrations of the inhibitor or antagonists include, but are not limited to less than about 100 mM, about 10 mM, about 1 mM, about 0.1 mM; optionally less than about 10 μM, about 1 μM, about 0.1 μM, about 0.01 μM about 0.001 μM, or about 0.0001 μM.

III.B. Formulation of Compositions

The GAPDHS biological activity modulating substances are adapted for administration as a pharmaceutical composition. Additional formulation and dose preparation techniques have been described in the art: see e.g., those described in U.S. Pat. No. 5,326,902 issued to Seipp et al. on Jul. 5, 1994, U.S. Pat. No. 5,234,933 issued to Marnett et al. on Aug. 10, 1993, and PCT International Publication Number WO 93/25521 of Johnson et al. published Dec. 23, 1993, the entire contents of each of which are herein incorporated by reference.

For treatment applications, an effective amount of a composition of the presently disclosed subject matter is administered to a subject. An “effective amount” is an amount of the composition sufficient to produce a measurable biological response, such as but not limited to a reduction in GAPDHS biological activity. Actual dosage levels of active ingredients in a composition of the presently disclosed subject matter can be varied so as to administer an amount of the active compound(s) that is effective to achieve the desired treatment response for a particular subject. The selected dosage level will depend upon a variety of factors including the activity of the composition, formulation, the route of administration, combination with other drugs or treatments, and the physical condition and prior medical history of the subject being treated. In some embodiments, a minimal dose is administered; and dose is escalated in the absence of dose-limiting toxicity. Determination and adjustment of a therapeutically effective dose, as well as evaluation of when and how to make such adjustments, are well known to those of ordinary skill in the art of medicine.

For the purposes described above, the identified substances can normally be administered systemically or partially, usually by oral, dermal, topical or parenteral administration. The term “parenteral” as used herein includes intravenous, intra-muscular, intra-arterial injection, or infusion techniques. Intravaginal and transdermal administration are also provided. The doses to be administered are determined depending upon age, body weight, symptom, the desired therapeutic effect, the route of administration, and the duration of the treatment, etc.; one of skill in the art of therapeutic treatment will recognize appropriate procedures and techniques for determining the appropriate dosage regimen for effective therapy. Various compositions and forms of administration are provided and are generally known in the art. Other compositions for administration include liquids for external use, and endermic liniments (ointment, etc.), suppositories and pessaries that comprise one or more of the active substance(s) and can be prepared by known methods.

Thus, the presently disclosed subject matter provides pharmaceutical compositions comprising a polypeptide, antibody or fragment thereof, small molecule or compound of the presently disclosed subject matter and a physiologically acceptable carrier. In some embodiments, a pharmaceutical composition comprises a compound discovered via the screening methods described herein.

A composition of the presently disclosed subject matter can be administered in dosage unit formulations containing standard, well-known nontoxic physiologically acceptable carriers, adjuvants, and vehicles as desired. Oral, topical, and transdermal administration (such as via a patch) are options for systemic delivery. If delivered in the female, an intravaginal foam or gel can be employed. Implantable rods that are used for administering some contraceptives are also an option for the presently disclosed subject matter.

Injectable preparations, for example sterile injectable aqueous or oleaginous suspensions, are formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol.

Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid find use in the preparation of injectables.

IV. Drug Screening Assays

A method of testing a candidate composition for GAPDHS modulation activity is also provided in accordance with the presently disclosed subject matter. By way of example and not limitation, through these methods, one can identify ligands or substrates that have a contraceptive effect and/or an effect on sperm motility. Of particular interest are screening methods for candidate compositions that have a low toxicity for human cells. Representative methods include biochemical-based methods and computer-based design methods (e.g. rational drug design).

A method of screening a candidate composition for an effect on reproduction (e.g. contraceptive activity) is thus disclosed. In some embodiments, the method comprises (a) contacting a GAPDHS with a candidate compound; (b) determining an effect of the candidate compound on a biological activity of the GAPDHS; and (c) determining whether the candidate compound has an effect on reproduction based on the effect of the candidate compound on a biological activity of the GAPDHS. An aspect of identifying a compound is that it should selectively affect GAPDHS, and not the GAPDH isozyme that is universally present in somatic cells. These enzymes are part of the glycolytic pathway, used by all cells in energy metabolism. Therefore, in some embodiments an inhibitor selectively affects the sperm enzyme GAPDHS, thus mitigating side effects in other tissues. Recombinant forms of both GAPDHS and the somatic enzyme, GAPDH, are disclosed herein, and an aspect of the inhibition assays disclosed herein is to identify a compound that inhibits GAPDHS, but not GAPDH, under a given set of conditions.

A method of screening a candidate composition for an effect on sperm motility is also disclosed. In some embodiments, the method comprises (a) contacting a GAPDHS with a candidate compound; (b) determining an effect of the candidate compound on a biological activity of the GAPDHS; and (c) determining whether the candidate compound has an effect on sperm motility based on the effect of the candidate compound on a biological activity of the GAPDHS. In some embodiments, a GAPDHS is a mouse Gapdhs. In some embodiments, a GAPDHS is a human GAPDHS.

Optionally, the GAPDHS is a recombinant GAPDHS. The contacting can be carried out in vitro and/or can be carried out by administering the candidate compound to a test subject. Target cells can be either naturally occurring cells known to contain GAPDHS or transformed cells produced in accordance with a process of transformation. The test samples can further comprise a cell or cell line that expresses a GAPDHS. Such cell lines can be mammalian (e.g. murine or human) or they can be from another organism. In some embodiments, a transgenic approach is employed to express the human form of GAPDHS in the knockout mice disclosed herein. These animals are used for in vivo tests of inhibitors on the human isozyme. In some embodiments, a target cell is a cell that has been engineered to not express a somatic form of GAPDH, and to express a GAPDHS instead.

A screening assay can provide a cell under conditions suitable for testing the modulation of biological activity of a GAPDHS. These conditions include, but are not limited to pH, temperature, tonicity, the presence of relevant metabolic factors (e.g., metal ions such as for example Ca⁺², growth factor, interleukins, or colony stimulating factors), and relevant modifications to the polypeptide such as glycosylation or prenylation. The GAPDHS polypeptide can be expressed and utilized in a prokaryotic or eukaryotic cell. U.S. Pat. Nos. 5,837,479; 5,645,999; 5,786,152; 5,739,278; and 5,352,660 also describe exemplary screening assays, and the entire contents of each are herein incorporated by reference.

A method for identifying a GAPDHS modulator is also disclosed. In some embodiments, the method comprises (a) providing atomic coordinates of a GAPDHS to a computerized modeling system; and (b) modeling a ligand that fits spatially into a binding pocket of the GAPDHS to thereby identify a GAPDHS modulator.

A method of modeling an interaction between a GAPDHS and a ligand is also disclosed. In some embodiments, the method comprises: providing a homology model of a target GAPDHS; providing atomic coordinates of a ligand; and docking the ligand with the homology model to form a GAPDH/ligand model.

In any of the foregoing embodiments, the GAPDHS can be human GAPDHS. In this case, the modified ligand is optionally designed to interact with one or more residues in human GAPDHS including, but not limited to N81, G82, F83, G84, R85, I86, G87, L89, N105, D106, P107, F108, C150, K151, E152, P153, E168, S169, T170, V172, Y173, L174, S175, A178, S193 P197, S223, C224, T225, H251, S252, Y253, T254, A255, T256, Q257, K258, S264, R265, A267, R269, D270, G271, I279, P280, A281, S282, T283, G284, A304, R306, P310, N388, E389, Y392, and S393 of SEQ ID NO: 6.

Additionally, the GAPDHS can be mouse Gapdhs. In this case, the modified ligand is optionally designed to interact with one or more residues of mouse Gapdhs including, but not limited to N111, G112, F113, G114, R115, I116, L119, N135, D136, P137, F138, C180, K181, D182, P183, E198, C199, T200, V202, Y203, L204, S205, A208, T223, P227, S253, C254, T255, H281, S282, Y283, T284, A285, T286, Q287, K288, S294, K295, D297, R299, G300, G301, I309, P310, S311, S312, T313, G314, A334, R336, P340, N418, E419, Y422, and S423 of SEQ ID NO: 2.

Additionally, the GAPDHS can be rat GAPDHS. In this case, the modified ligand is optionally designed to interact with one or more residues of rat GAPDHS including, but not limited to N105, G106, F107, G108, R109, I110, L113, N129, D130, P131, F132, C174, K175, E176, P177, E192, A193, T194, V196, Y197, L198, S199, A202, T217, P221, S247, C248, T249, H275, A276, Y277, T278, A279, T280, Q281, K282, S288, K289, D291, R293, G294, G295, I303, P304, S305, S306, T307, G308, A328, R330, P334, N412, E413, Y416, and S417 of SEQ ID NO: 4.

Of course, the foregoing listings of amino acid residues are meant to be representative and non-limiting examples. Additional residues, including neighboring residues and other residues as would be apparent to one of ordinary skill in the art, are meant to fall within the scope of the presently disclosed subject matter as well. The interaction can be selected from the group consisting of a van der Waals interaction, a hydrophobic interaction, hydrogen bonding, and combinations thereof.

In some embodiments, a method of designing a modulator of a GAPDHS comprises (a) selecting a candidate GAPDHS ligand; (b) determining which amino acid or amino acids of the GAPDHS interact with the ligand using a three-dimensional model of a GAPDHS; (c) identifying in a biological assay for GAPDHS activity a degree to which the ligand modulates the activity of the GAPDHS; (d) selecting a chemical modification of the ligand wherein the interaction between the amino acids of the GAPDHS and the ligand is predicted to be modulated by the chemical modification; (e) synthesizing a ligand having the chemical modified to form a modified ligand; (f) identifying in a biological assay for GAPDHS activity a degree to which the modified ligand modulates the biological activity of the GAPDHS; and (g) comparing the biological activity of the GAPDHS in the presence of modified ligand with the biological activity of the GAPDHS in the presence of the unmodified ligand, whereby a modulator of a GAPDHS is designed.

In some embodiments, the GAPDHS can be human GAPDHS. In this case, the modified ligand is optionally designed to interact with one or more residues in human GAPDHS including, but not limited to N81, G82, F83, G84, R85, I86, G87, L89, N105, D106, P107, F108, C150, K151, E152, P153, E168, S169, T170, V172, Y173, L174, S175, A178, S193 P197, S223, C224, T225, H251, S252, Y253, T254, A255, T256, Q257, K258, S264, R265, A267, R269, D270, G271, I279, P280, A281, S282, T283, G284, A304, R306, P310, N388, E389, Y392, and S393 of SEQ ID NO: 6. Additionally, the GAPDHS can be mouse Gapdhs. In this latter case, the modified ligand is optionally designed to interact with one or more residues of mouse Gapdhs including, but not limited to N111, G112, F113, G114, R115, I116, L119, N135, D136, P137, F138, C180, K181, D182, P183, E198, C199, T200, V202, Y203, L204, S205, A208, T223, P227, S253, C254, T255, H281, S282, Y283, T284, A285, T286, Q287, K288, S294, K295, D297, R299, G300, G301, I309, P310, S311, S312, T313, G314, A334, R336, P340, N418, E419, Y422, and S423 of SEQ ID NO: 2. Additionally, the GAPDHS can be rat GAPDHS. In this case, the modified ligand is optionally designed to interact with one or more residues of rat GAPDHS including, but not limited to N105, G106, F107, G108, R109, I110, L113, N129, D130, P131, F132, C174, K175, E176, P177, E192, A193, T194, V196, Y197, L198, S199, A202, T217, P221, S247, C248, T249, H275, A276, Y277, T278, A279, T280, Q281, K282, S288, K289, D291, R293, G294, G295, I303, P304, S305, S306, T307, G308, A328, R330, P334, N412, E413, Y416, and S417 of SEQ ID NO: 4. The interaction can be selected from the group consisting of a van der Waals interaction, a hydrophobic interaction, hydrogen bonding, and combinations thereof. The method can further comprise repeating the steps if the biological activity of the GAPDHS in the presence of the modified ligand varies from the biological activity of the GAPDHS in the presence of the unmodified ligand.

The design of candidate or test substances, also referred to as “compounds” or “candidate compounds”, which bind to GAPDHS and/or modulate GAPDHS-mediated activity according to the presently disclosed subject matter generally involves consideration of two factors. First, the compound must be capable of chemically and structurally associating with GAPDHS. Non-covalent molecular interactions important in the association of a GAPDHS with its substrate include hydrogen bonding, van der Waals interactions, and hydrophobic interactions. The interaction between an atom of a GAPDHS amino acid and an atom of a ligand/substrate can be made by any force or attraction described in nature. Usually the interaction between the atom of the amino acid and the ligand will be the result of hydrogen bonding interaction, charge interaction, hydrophobic interaction, van der Waals interaction, or dipole interaction. In the case of the hydrophobic interaction, it is recognized that this is not a per se interaction between the amino acid and ligand, but rather the usual result, in part, of the repulsion of water or other hydrophilic group from a hydrophobic surface. Reducing or enhancing the interaction of the GAPDHS and a ligand can be measured by calculating or testing binding energies, either computationally or using thermodynamic or kinetic methods known in the art.

Second, the compound must be able to assume a conformation that allows it to associate with a GAPDHS. Although certain portions of the compound will not directly participate in this association with a GAPDHS, those portions can still influence the overall conformation of the molecule. This influence on conformation, in turn, can have a significant impact on potency. Such conformational requirements include the overall three-dimensional structure and orientation of the chemical entity or compound in relation to all or a portion of a binding site of the GAPDHS, or the spacing between functional groups of a compound comprising several chemical entities that directly interact with a GAPDHS.

Chemical modifications can enhance or reduce interactions of an atom of a GAPDHS amino acid and an atom of a GAPDHS ligand. Steric hindrance can be a common approach for changing the interaction of a GAPDHS binding site with an activation domain. Chemical modifications are introduced in some embodiments at C—H, C—, and C—OH positions in a ligand, where the carbon is part of the ligand structure that remains the same after modification is complete. In the case of C—H, C could have 1, 2, or 3 hydrogens, but usually only one hydrogen will be replaced. The H or OH can be removed after modification is complete and replaced with a desired chemical moiety.

The modulatory or binding effect of a chemical compound on a GAPDHS can be analyzed prior to its actual synthesis and biochemical testing by the use of computer modeling techniques that employ the coordinates of a GAPDHS. If the structure of the given compound suggests insufficient interaction and association between it and a GAPDHS, synthesis and testing of the compound is obviated. However, if computer modeling indicates a strong interaction, the molecule can then be synthesized and tested for its ability to bind and modulate the activity of a GAPDHS. In this manner, synthesis of unproductive or inoperative compounds can be avoided.

A modulatory or other binding compound of a GAPDHS can be computationally evaluated and designed via a series of steps in which chemical entities or fragments are screened and selected for their ability to associate with an individual binding site or other area of a GAPDHS polypeptide of the presently disclosed subject matter and to interact with the amino acids disposed in the binding sites.

Interacting amino acids forming contacts with a ligand and the atoms of the interacting amino acids are usually 2 to 4 angstroms away from the center of the atoms of the ligand. Generally these distances are determined by computer as discussed herein and in McRee, 1993, however distances can be determined manually once the three dimensional model is made. More commonly, the atoms of the ligand and the atoms of interacting amino acids are 3 to 4 angstroms apart. A ligand can also interact with distant amino acids, after chemical modification of the ligand to create a new ligand. Distant amino acids are generally not in contact with the ligand before chemical modification. A chemical modification can change the structure of the ligand to make a new ligand that interacts with a distant amino acid usually at least 4.5 angstroms away from the ligand. Distant amino acids rarely line the surface of the binding cavity for the ligand, as they are too far away from the ligand to be part of a pocket or surface of the binding cavity.

A compound designed or selected as binding to a GAPDHS can be further computationally optimized so that in its bound state it would lack repulsive electrostatic interaction with the target polypeptide. Such non-complementary (e.g., electrostatic) interactions include repulsive charge-charge, dipole-dipole, and charge-dipole interactions. Specifically, the sum of all electrostatic interactions between the modulator and the polypeptide when the modulator is bound to a GAPDHS make a neutral or favorable contribution to the enthalpy of binding.

One of several methods can be used to screen chemical entities or fragments for their ability to associate with a GAPDHS and, more particularly, with the individual binding sites of a GAPDHS. This process can begin by visual inspection of, for example, a binding site on a computer screen. Selected fragments or chemical entities can then be positioned in a variety of orientations, or docked, within an individual binding site of a GAPDHS as defined herein. Docking can be accomplished using software programs such as those available under the trade names QUANTA™ (available from Accelrys Inc, San Diego, Calif., United States of America) and SYBYL® (available from Tripos, Inc., St. Louis, Mo., United States of America), followed by energy minimization and molecular dynamics with standard molecular mechanics force fields, such as CHARMM® (Brooks et al., 1983) and AMBER 5 (Case et al., 1997; Pearlman et al., 1995).

Specialized computer programs can also assist in the process of selecting fragments or chemical entities. These include:
1. GRID™ program, version 17 (Goodford, 1985), which is available from Molecular Discovery Ltd. of Oxford, United Kingdom;
2. MCSS™ program (Miranker & Karplus, 1991), which is available from Accelrys Inc, San Diego, Calif., United States of America;
3. AUTODOCK™ 3.0 program (Goodsell & Olsen, 1990), which is available from the Scripps Research Institute, La Jolla, Calif., United States of America;
4. DOCK™ 4.0 program (Kuntz et al., 1992), which is available from the University of California, San Francisco, Calif., United States of America;
5. FLEX-X™ (see Rarey et al., 1996) and SITE ID™ programs, which are available from Tripos, Inc. of St. Louis, Mo., United States of America;
6. MVP program (Lambert, 1997 at 243-303); and
7. LUDI™ program (Bohm, 1992), which is available from Accelrys Inc, San Diego, Calif., United States of America.

Once suitable chemical entities or fragments have been selected, they can be assembled into a single compound or modulator. Assembly can proceed by visual inspection of the relationship of the fragments to each other on the three-dimensional image displayed on a computer screen in relation to the structure coordinates of GAPDHS. Manual model building using software such as QUANTA™ or SYBYL® typically follows.

Useful programs to aid one of ordinary skill in the art in connecting the individual chemical entities or fragments include:
1. CAVEAT™ program (Bartlett et al., 1989), which is available from the University of California, Berkeley, Calif., United States of America;
2. 3 D Database systems, such as MACCS-3D™ system program, which is available from MDL Information Systems of San Leandro, Calif., United States of America. This area is reviewed in Martin, 1992.
3. HOOK™ program (Eisen et al., 1994), which is available from Accelrys Inc, San Diego, Calif., United States of America.

Instead of proceeding to build a GAPDHS modulator in a step-wise fashion one fragment or chemical entity at a time as described above, modulatory or other binding compounds can be designed as a whole or de novo using a homology model as disclosed herein. Applicable methods can employ the following software programs:
1. LUDI™ program (Bohm, 1992), which is available from Accelrys Inc, San Diego, Calif., United States of America;
2. LEGEND™ program (Nishibata & Itai, 1991); and
3. LEAPFROG™, which is available from Tripos, Inc., St. Louis, Mo., United States of America.

Other molecular modeling techniques can also be employed in accordance with the presently disclosed subject matter. See e.g., Cohen et al., 1990; Navia & Murcko, 1992; and U.S. Pat. No. 6,008,033 to Abdel-Mequid et al., all of which are incorporated herein by reference.

Once a compound has been designed or selected by the above methods, the efficiency with which that compound can bind to a GAPDHS can be tested and optimized by computational evaluation. By way of a particular example, a compound that has been designed or selected to function as a GAPDHS modulator can traverse a volume not overlapping that occupied by the binding site when it is bound to its native ligand. Additionally, an effective GAPDHS modulator can demonstrate a relatively small difference in energy between its bound and free states (i.e., a small deformation energy of binding). Thus, the most efficient GAPDHS modulators can be designed with a deformation energy of binding of in some embodiments not greater than about 10 kcal/mole, and in some embodiments not greater than 7 kcal/mole. It is possible for GAPDHS modulators to interact with the polypeptide in more than one conformation that is similar in overall binding energy. In those cases, the deformation energy of binding is taken to be the difference between the energy of the free compound and the average energy of the conformations observed when the modulator binds to the polypeptide.

A compound designed or selected as binding to a GAPDHS can be further computationally optimized so that in its bound state it would preferably lack repulsive electrostatic interaction with the target polypeptide. Such non-complementary (e.g., electrostatic) interactions include repulsive charge-charge, dipole-dipole, and charge-dipole interactions. Specifically, the sum of all electrostatic interactions between the modulator and the polypeptide when the modulator is bound to a GAPDHS preferably make a neutral or favorable contribution to the enthalpy of binding.

Specific computer software is available in the art to evaluate compound deformation energy and electrostatic interaction. Examples of programs designed for such uses include:
1. GAUSSIAN 98™, which is available from Gaussian, Inc. of Pittsburgh, Pa., United States of America;
2. AMBER™ program, version 6.0, which is available from the University of California, San Francisco, Calif., United States of America;
3. QUANTA™ program, which is available from Accelrys Inc, San Diego, Calif., United States of America;
4. CHARMM® program, which is available from Accelrys Inc, San Diego, Calif., United States of America; and
4. INSIGHT II® program, which is available from Accelrys Inc, San Diego, Calif., United States of America.

These programs can be implemented using a suitable computer system. Other hardware systems and software packages will be apparent to those skilled in the art after review of the disclosure of the presently disclosed subject matter presented herein.

Once a GAPDHS modulating compound has been optimally selected or designed, as described above, substitutions can then be made in some of its atoms or side groups in order to improve or modify its binding properties. Generally, initial substitutions are conservative, i.e., the replacement group will have approximately the same size, shape, hydrophobicity, and charge as the original group. Components known in the art to alter conformation are avoided. Such substituted chemical compounds can then be analyzed for efficiency of fit to a GAPDHS binding site using the same computer-based approaches described in detail above.

The methods of the presently disclosed subject matter can also be used to suggest possible chemical modifications of a compound that might reduce or minimize its effect on GAPDHS. This approach can be useful in drug discovery projects aiming to find compounds that modulate the activity of some other target molecule, where modulation of GAPDHS activity is an undesirable side effect. This approach is useful in engineering GAPDHS activity out of other, non-drug molecules. Humans and other animals are exposed to a wide range of different chemical compounds, some of which can act on GAPDHS in an undesirable manner. Such a compound could be predicted computationally using molecular docking software, as discussed herein. The structure could be examined by computer graphics to suggest chemical modifications that would minimize the tendency to bind to GAPDHS. For example, a region of the molecule that binds to a lipophilic region of the GAPDHS binding site could be modified to make it more polar, thus reducing its tendency to bind to GAPDHS. Alternatively, a polar group of the compound that makes a hydrogen bonding interaction with GAPDHS could be identified and modified to an alternative group that fails to make the hydrogen bond. Appropriate chemical modifications can be chosen such that the desirable properties and behavior of the compound would be retained.

V. Other Applications

Another area where monitoring GAPDHS activity can be employed is in the assessment of male infertility. Many males produce normal numbers of sperm with normal morphology, but are nonetheless infertile. Many of these males produce sperm with impaired motility. For those males who produce sperm with impaired motility, it is possible to use the techniques disclosed herein to measure GAPDHS activity through the use of compounds that interact with GAPDHS, including but not limited to compounds that selectively interact with GAPDHS. In some embodiments, these compounds can be used to determine whether there are mutations in the amino acids that define pockets in the GAPDHS, in that the compounds that should interact, can be observed not to interact in the presence of a mutation. For those individuals where GAPDHS activity is reduced or absent, subsequent studies would look for mutations in the GAPDHS gene, taking advantage of the nucleic acid and amino acid sequences disclosed herein. The ability to identify specific genetic mutations would be beneficial to clinicians and patients because doing so would help avoid many months of costly treatments that are taxing both physically and psychologically for the patients.

Some mutations that would be detected would be expected to encode polypeptides with lower than optimal enzymatic activity. Using the methods and compositions disclosed herein, modulators could be modeled that would be predicted to interact with the specific mutations detected. These modulators could then be designed to enhance the activity of the mutant polypeptide in an attempt to restore normal sperm function (e.g. motility and the ability to fertilize an ovum). In the study of mutations identified in human GAPDHS, transgenic mice are constructed to express the variants. The effects of GAPDHS modulators are then tested on these variants, to identify activators that can be used to treat infertility.

### EXAMPLES

The following Examples have been included to illustrate representative and exemplary modes of the presently disclosed subject matter. In light of the present disclosure and the general level of skill in the art, those of skill will appreciate that the following Examples are intended to be exemplary only and that numerous changes, modifications, and alterations can be employed without departing from the spirit and scope of the presently disclosed subject matter.

### Example 1

### Sequence Alignments and Homology Modeling of GAPDHS and Gapdhs Compared to Human Muscle GAPDH

Alignments for development of the GAPDHS and GAPDHS protein models (FIGS. 1A-1D) were performed using the GCG® WISCONSIN PACKAGES (available from Accelrys, Inc., San Diego, Calif., United States of America) BestFit and Pileup program using the Blosum 62 and Pam 250 matrix algorithms and the Clustal W algorithm. The GAPDHS structural model excluded the N-terminal proline-rich region (residues 1-103) and began with arginine 104 (R104). The structural model developed for GAPDHS also excluded the N-terminal proline-rich region (residues 1-73) and began with arginine 74 (R74). In the alignments on which the models were based, there is one deletion (lysine 26 in GAPDH) and one insertion (proline 243 in GAPDHS; proline 213 in GAPDHS).

Initial homology models were constructed for GAPDHS and GAPDHS by fitting their amino acid sequences and protein backbone to the coordinates for the human muscle GAPDH crystal structure deposited in the RCSB Protein Databank (RCSB PDB entry: G3PD), available through the website of the Research Collaboratory for Structural Bioinformatics (RCSB). Excluding the proline-rich N-terminal domains, the Gapdhs sequence was 69% identical and the GAPDHS sequence was 68% identical to this GAPDH sequence (GCG Bestfit program Blosum 62 and Pam250 comparison matrices). In addition, the Gapdhs sequence was 86% similar and 82% identical to the GAPDHS sequence when the N-terminal proline-rich domains were included (GCG Bestfit program Blosum 62 and Pam250 comparison matrices). Homology modeling, molecular dynamics and energy minimization calculations, substrate and inhibitor docking, and other graphical manipulations were performed using the Accelrys Molecular Simulations (MSI) Software package (Software Modules: Biopolymer, Builder, Insight II, Discover_{—}3.0 and Affinity). Discontinuities were resolved in the homology models by minimizing the structures using the Accelrys MSI Discover_{—}3.0 molecular dynamics program. Coordinates for the structure of NAD in the cofactor-binding pocket of human muscle GAPDH were obtained from the solved x-ray structure coordinates in the RCSB Protein Databank (RCSB PDB entry: 3GPD) and translated to the modeled structures for Gapdhs and GAPDHS. Potentials were set for all the atoms using the Lifson and Hagler CVFF force field (Hagler et al., 1979). No cross and no morse terms were included. Both Gapdhs and GAPDHS were modeled as dimers omitting the proline-rich N-terminal sequences (Gapdhs dimer in vacuo 672 residues, 10,332 atoms; GAPDHS dimer in vacuo 675 residues, 10,398 atoms). Following in vacuo minimization and dynamics, the Gapdhs (and GAPDHS) monomers were both solubilized in a 5 Å sphere of water (1730 water molecules, 5,190 atoms) and minimized. Solvated minimization and dynamics of the homology modeled Gapdhs and GAPDHS were performed using non-periodic boundary conditions with a distance-dependent dielectric and the cell multipole method for non-bonded interactions (Coulombic and van der Waals).

### Example 2

### GAPDHS and Gapdhs Models: Binding Pockets for the Substrate and Cofactor

Sequence alignments (see FIGS. 1A-1D) identified 48 amino acids identical in mouse Gapdhs and human GAPDHS that are significantly different, in chemical and physical properties, from the residues at corresponding positions in mouse and human GAPDH. See FIGS. 1A-1D. Based on the homology models developed in Example 1, eight of these residues that are located 20 Å or less from the substrate-binding pocket differ sufficiently in chemical and structural properties to affect conformational changes within the substrate-binding domain and/or the NAD cofactor-binding domain. See Table 2. The spatial arrangements of these eight residues in human GAPDH and GAPDHS are shown in FIG. 3. One of the most significant changes in the region of the protein containing the catalytic domain is the replacement of a small, uncharged glycine residue (G192 in human GAPDH; G191 in mouse Gapdh (GENBANK® Accession No. NP_{—}032110)) by a large, positively charged lysine (K295) in Gapdhs or arginine (R265) in GAPDHS. This residue is of particular significance as the corresponding residue in trypanosome Gapdh interacts with NAD analogs that disrupt glycolysis and are structure-based designed drugs for African sleeping sickness, leishmaniasis, and Chagas disease (Aronov et al., 1998; Aronov et al., 1999; Verlinde et al., 1994; Bressi et al., 2000; Bressi et al., 2001).

The other significant changes in the catalytic domain are the replacement of a hydrophobic isoleucine (1180 in human GAPDH; 1179 in mouse Gapdh) by a larger, aromatic tyrosine bearing an ionizable hydroxyl group (Y283 in mouse Gapdhs; Y253 in GAPDHS), and the replacement of a small, hydrophobic alanine (A179 in human GAPDH; A178 in mouse Gapdh) by a larger hydrophilic serine (S282 in mouse Gapdhs; S252 in GAPDHS). Several residues in the catalytic and NAD cofactor-binding domains are potential phosphorylation sites (T102 in human GAPDH, S205 and S282 in mouse Gapdhs; Y173 and S252 in human GAPDHS) and might be involved in signaling or interactions with other molecules.

There are five significant residue changes in the region of the protein containing the NAD cofactor-binding domain. They include changes in two hydrophobic residues, an alanine (A124 in mouse Gapdh; A125 in human GAPDH) that is replaced by a highly constraining proline residue (P227 in mouse Gapdhs; P197 in GAPDHS), and a phenylalanine (F100 in mouse Gapdh; F101 in human GAPDH) that is replaced by a hydrophilic, ionizable tyrosine (Y203 in mouse Gapdhs; Y173 in GAPDHS). In addition, a large, positively charged lysine (K105 in mouse Gapdh; K106 in human GAPDH) is replaced by a small uncharged alanine (A208 in mouse Gapdhs; A178 in GAPDHS), a hydrophilic threonine (T101 in mouse Gapdh; T102 in human GAPDH) is replaced with a hydrophobic leucine (L204 in mouse Gapdhs; L174 in GAPDHS), and a larger threonine residue (T102 in mouse Gapdh; T103 in human GAPDH) is replaced by a smaller serine residue (S205 in mouse Gapdhs; S175 in GAPDHS). Although all of these residues are located in the NAD cofactor-binding domain, the residue Cα carbons are at distances of approximately 12-20 angstroms from the substrate-binding pocket.

Other residues in mouse and human GAPDH are replaced by dissimilar residues in mouse Gapdhs and human GAPDHS. These also might have significant structural or functional effects. There are three positions where hydrophobic phenylalanine residues in human GAPDH (F55, F101, F317) are replaced by hydrophilic and ionizable tyrosine residues in mouse Gapdhs (Y157, Y203, Y420) and human GAPDHS (Y127, Y173, Y390). While F55 and F101 are highly conserved with the corresponding residues of GAPDH in other species, F317 is less well conserved. In addition, tyrosine is more highly water-soluble and reactive than phenylalanine and is a potential phosphorylation site. There are also substitutions for three of the threonine residue in GAPDH (T58, T72, and T91) by serine residues in GAPDHS(S130, S146, S163) and by asparagine residues in mouse Gapdhs (N160, N176, N193). However, these residues are not highly conserved between human GAPDH and GAPDH of other species.

### Example 3

### GAPDHS and Gapdhs Models: Additional Features of the Active Site

The GAPDHS and Gapdhs homology models generated in Example 1 were compared systematically with the crystal structure of human GAPDH. The backbones of these three enzymes are superimposed in FIG. 3, and side chains with highly significant differences are highlighted. Structural changes in the size and shape in the substrate-binding pocket and NAD cofactor-binding pocket of GAPDHS and Gapdhs caused by substitutions for residues in GAPDH were examined critically. The root mean square deviation (RMSD; a quantitative measure of the overall difference between the structures) between the Cα backbone structure of the GAPDHS and Gapdhs models and the structure of human GAPDH is 2.385 Å and 2.380 Å respectively, while the RMSD between the Cα backbone structure of the GAPDHS and Gapdhs models is 0.96 Å. This indicates that the modeled structures of GAPDHS and Gapdhs are very similar but differ significantly from the structure of somatic GAPDH.

The side chain of the catalytic cysteine (Cys 151, GAPDH; Cys 254, Gapdhs; Cys 224, GAPDHS) extends much further into the binding pocket in GAPDH than in mouse Gapdhs and GAPDHS, contributing to the alteration in the shape of the substrate-binding pocket. Although the sequence that contains the catalytic cysteine, NASCTTNCL (SEQ ID NO: 9; corresponding to residues 148-156 in GAPDH; residues 251-259 in mouse Gapdhs; and residues 221-229 in GAPDHS), is highly conserved in somatic and spermatogenic cell isoforms, the backbone conformation of this sequence in mouse Gapdhs or GAPDHS can not be superimposed entirely on GAPDH.

Arg 306 is 4.28 Å from the substrate phosphate-binding site in the GAPDHS model, while the corresponding residue in GAPDH (Arg 233) is 6.11 Å from the substrate phosphate-binding site. This Arg is a highly invariant residue, participates in determining substrate specificity, and is essential for catalytic activity (Duée et al., 1996). When a conformational change occurs during enzyme catalysis, this Arg residue forms a hydrogen bond with the phosphate group of the substrate. Changes in the location of the Arg would effect salt bridge formation with arginine and hydrogen bond formation in the active site. This Arg brings a positive charge to the active center of the complex (Murthy et al., 1980) and the closer location of this residue to the substrate-binding site in GAPDHS and Gapdhs is significant. Other GAPDHS residues surrounding the substrate-binding pocket (281-289 and 302-306) are super-imposable on the corresponding residues in GAPDH (208-216 and 229-233) and exhibit similar backbone conformations.

There are several significant differences between the spermatogenic and somatic isoforms in the conformations assumed by some of the protein loops less than 20 Å from the substrate-binding pocket. These differences in loop conformations contribute to a difference in the shapes of the substrate-binding and cofactor-binding pockets. One of the significant differences near the substrate-binding pocket is the conformation of a loop in mouse Gapdhs (residues 293-305 of SEQ ID NO: 2) and GAPDHS (residues 263-275 of SEQ ID NO: 6) and the corresponding loop in GAPDH (residues 190-202 of SEQ ID NO: 8; see FIGS. 1A-1D). The backbone conformation of A267 in GAPDHS is particularly different than the corresponding residue in GAPDH (L174) and the Arg residue in this loop in mouse Gapdhs (Arg 299) or GAPDHS (Arg 269) is not congruent with the corresponding residue in GAPDH (Arg 196).

| | | |
|---|---|---|
| Gapdhs: | PSKKDWRGGRGAH | (SEQ ID NO: 10) |
| GAPDHS: | PSRKAWRDGRGAH | (SEQ ID NO: 11) |
| GAPDH: | PSGKLWRGGRGAA | (SEQ ID NO: 12) |

The conformation of another loop (residues 203-209 in mouse Gapdhs, SEQ ID NO: 2; residues 173-179 in GAPDHS, SEQ ID NO: 6; and residues 101-106 in GAPDH, SEQ ID NO: 8) is also significantly different in the two isoforms. This sequence is initiated with a Y in mouse Gapdhs and GAPDHS that is a highly conserved F in all other GAPDH sequences. It is also one of the eight amino acids required for NAD cofactor-binding. In particular, the backbone conformation is significantly different due to the replacement of residue T102 in GAPDH with residue L174 in (GAPDHS) and L204 (in mouse Gapdhs).

| | | |
|---|---|---|
| Gapdhs: | YLSIEA | (SEQ ID NO: 13) |
| GAPDHS: | YLSIQA | (SEQ ID NO: 14) |
| GAPDH: | FTTMEK | (SEQ ID NO: 15) |

The differences in the conformation of the loops show below arise primarily due to the significantly different conformation of the Pro 197 in GAPDHS corresponding to Ala 125 in GAPDH. Additionally, the initial proline residues in this protein loop in GAPDHS(P195), Gapdhs (P225), and GAPDH (P123) are not superimposable when the structures are overlayed. This is due to the constrained nature of the proline residue and its dramatic effect on the protein structure backbone conformation.

| | | |
|---|---|---|
| Gapdhs: | PSPDA | (SEQ ID NO: 16) |
| GAPDHS: | PSPDA | (SEQ ID NO: 16) |
| GAPDH: | PSADA | (SEQ ID NO: 17) |

Thr 249 in GAPDHS (Thr 279 in Gapdhs) in the protein loop shown below, is not well overlapped with the backbone conformation of Thr 176 in GAPDH. This Thr is near two significant residues, which are close to the active site Cys: Ser 252, and Tyr 253 (GAPDHS). The first residue in this loop (M247) has a conformation that is not superimposable in these structures. Thr 254 (GAPDHS) following the SY in this sequence is the Thr reported to confer substrate specificity in this binding site (Duée et al., 1996). Residues Thr 181, Arg 233, and Thr 210 of GAPDHS have been reported as conferring substrate specificity.

| | | |
|---|---|---|
| Gapdhs | MTTVHSYT | (SEQ ID NO: 18) |
| GAPDHS: | MTTVHSYT | (SEQ ID NO: 18) |
| GAPDH: | MTTVHAIT | (SEQ ID NO: 19) |

### Example 4

### Docking Substrates and Inhibitors to GAPDH, GAPDHS, and Gapdhs

Using the homology models constructed in Example 1, the native substrate glyceraldehyde 3-phosphate (G3P), and competitive substrate inhibitor (S)-3-chlorolacetaldehyde were individually docked into the substrate-binding pockets of GAPDH and the resulting structural configurations used to develop the docking models for GAPDHS and Gapdhs. Structures of G3P and (S)-3-chlorolacetaldehyde were built and minimized using the Molecular Simulations Biopolymer, Builder, and Discover_{—}3 software modules (Accelrys, Inc., San Diego, Calif., United States of America). The Discover_{—}3.0 CVFF force field was used for all calculations with distant dependent dielectric, non-periodic boundary conditions, and the cell multipole method for non-bonded interactions.

The enzyme active site was defined as the region that includes atoms within 8 Å from the binding site in GAPDH for the phosphate moiety of the substrate. This definition of the active site would be as inclusive as possible of all the nearest neighbors involved in direct bonded interactions with the ligand. This configuration was used to develop the GAPDHS and Gapdhs models. A Monte Carlo docking procedure (Accelrys MSI Affinity program; Luty et al., 1995) was used which kept bulk enzyme atoms, defined not to be part of the active site, rigid during the docking process. Only the enzyme active site and ligand atoms were permitted to move and retain flexibility. The docking algorithm used did not include solvation terms, and non-bonded interactions were computed using the cell multipole method (Molecular Simulations, Discover_{—}3). The ligand atoms were randomly rotated and translated and the energy of the substrate-binding site evaluated. If it fell within 1000 Kcal/mole of the previous structure it was subjected to 300 steps of energy minimization and accepted or rejected based on an energy range test. Structures within 10 kilocalories (Kcal) per mole of the lowest energy state structure were accepted.

Calculations were also performed to measure the volume of the substrate-binding pocket for comparison between GAPDH, GAPDHS, and Gapdhs. Approximate volumes were determined by calculating the molecular surface area buried due to the binding of the native substrate and inhibitor. The accessible molecular surface of the ligand and enzyme were calculated with and without a ligand docked in the active site, using the algorithm developed by Gert Vriend and Roland Krause present in the program WHAT IF (Vriend, 1990). Measurements were determined for the accessible surface area of the residues within 8 Å of the phosphate-binding site that are identical in GAPDHS and Gapdhs, but different from the corresponding residues conserved in GAPDH, using both the algorithm present in WHAT IF (based on the Connolly method; Connolly, 1983), and in the AccelrysAccess_Surf program (based on the Lee & Richards algorithm; Lee & Richards, 1971).

GAPDH undergoes a significant conformational change upon binding the adenosine moiety of NAD to become a catalytically active holoenzyme. This conformational change optimizes positioning of catalytic residues C151 and H178, and the anion-(phosphate) binding site. Hydride transfer occurs between C4 of the nicotinamide and thiohemicetal intermediate. NAD is in an open extended conformation when bound to the enzyme and N315 fixes the plane of the nicotinamide ring of NAD through H bonding, involving three structurally conserved waters.

The conformation of the substrate-binding pockets of GAPDH and GAPDHS were compared with (S)-3-chlorolacetaldehyde bound. The model, in conjunction with native substrate and inhibitor docking studies, predicts specific structural differences that might be responsible for the selective inhibition of GAPDHS by chloro-analogs of the native substrate. The eight residues in GAPDHS and Gapdhs that replace highly conserved residues in GAPDH and are located 20 angstroms or less from the substrate-binding pocket (see Table 2) significantly alter the shape and size of the substrate-binding and NAD cofactor-binding sites.

The Connolly accessible molecular surface areas (probe sphere radius=1.0 Å) were calculated for the GAPDH and GAPDHS substrate-binding pockets in the presence and absence of the docked inhibitor. The program WHAT IF by Vriend was used to calculate the molecular surface area buried by the ligand. The difference in the exposed molecular surface area on binding ligand was larger in the case of GAPDHS than GAPDH. This means that the ligand buried and covered more of the exposed molecular surface area in the spermatogenic isoform, GAPDHS, on binding. This means that more of the molecular surfaces of the inhibitor and substrate-binding pocket are in use when the inhibitor is docked in GAPDHS than in GAPDH.

### Example 5

### The Proline-rich N-terminal Domains of GAPDHS and Gapdhs

The proline-rich N-terminal domains of GAPDHS and Gapdhs were modeled separately from the rest of the protein. The solved protein structure found to be most similar to this domain by BLAST was the HTLVII (human T-Cell Leukemia Virus Type II) matrix protein (RCSB PDB entry: 1JVR). The proline-rich domain of GAPDHS (residues 33-67) shares 54.22% sequence similarity and 48.57% sequence identity with residues 90-127 of the HTLVII matrix protein. The proline-rich domain of Gapdhs (residues 36-77) shares 51.3% sequence similarity and 46.2% sequence identity respectively with the HTLVII matrix protein (residues 93-131). The Molecular Simulations Homology and Discover_{—}3 packages were used for the homology modeling with the same parameters as described above for the GAPDHS and Gapdhs models.

The HTLVII (human T-Cell Leukemia Virus Type II) proline-rich matrix protein (RCSB PDB entry: 1JVR) was used as the template structure for modeling the proline-rich N-terminal domains of GAPDHS and Gapdhs. The viral protein contributes to the structure of the outer coat and the proline-rich domains of GAPDHS and Gapdhs appear to provide structural attachment of the proteins to the fibrous sheath. Sequence alignments between the N-terminal proline-rich region of GAPDHS and the HTLVII Matrix protein were used in developing the homology models for this region, as well as a structure of the proline-rich domain. The deduced model suggests that this proline rich domain is a flexible extended arm-like linker connecting the central GAPDHS and Gapdhs ligand binding domains to the sperm fibrous sheath.

The first 19 residues in the rat Gapdhs (MSRRDVVLTNVTVVQLRRD; SEQ ID NO: 20), mouse Gapdhs (MSRRDWLTNVTVVQLRRD; SEQ ID NO: 20) and human GAPDHS (MSKRDIVLTNVTVVQLLRQ; SEQ ID NO: 21) are highly conserved between species, yet are unique and are not seen in the N-terminus of any other identified proteins. In addition, the mouse and rat Gapdhs also have a CP sequence repeat pattern (repeated 6 times in the mouse form and 7 times in the rat form) inserted in the N-terminal polyproline region that is absent in the human GAPDHS. A search of the known sequence database identified two other proteins, a low voltage-activated T-type calcium channel alpha-1 subunit from Rattus norvegicus (GENBANK® Accession Nos. MD17796 (protein) and AF086827 (cDNA)), and the mitochondrial capsule selenoprotein from Rattus norvegicus (GENBANK® Accession Nos. CAA61138 (protein) and X87883 (cDNA) and mouse sperm (GENBANK® Accession No. P15265 (protein)). Both of these proteins are sperm-associated proteins, as the mitochondrial capsule protein called a selenoprotein is present mainly in the flagellum, surrounding the mitochondria in the middle piece. In addition, they are all rodent sequences. Therefore the CP repeat might have a specific function associated exclusively with rodent sperm.

### Example 6

### Identification of GAPDHS Ligands

Additional structures of the mouse and human GAPDH and GAPDHS molecules were modeled for use in ligand docking studies. One crystal structure of human GAPDH is available, but it is a low resolution structure from twinned crystals determined in 1983 and, therefore, likely of low quality. Higher resolution structures of GAPDH from other species are now available and were used to judge the quality of the original human GAPDH structure. The structural comparisons highlighted regions where the quality of the human GAPDH structure was questionable. These regions corresponded to areas of high sequence and structural conservation among all of the species, except for the human GAPDH structure. It was determined that the best structures for the docking studies would come from homology modeling based on the closely-related lobster GAPDH structure. See Shen et al., 2000. These structures were modeled using the INSIGHT II Molecular Modeling System from Accelrys, Inc. (San Diego, Calif., United States of America).

Ligands from a small molecule database were docked into and near the active site of GAPDHS using the FlexX algorithm of the SYBYL® 6.9 Molecular Modeling Environment (Tripos, Inc., St. Louis, Mo., United States of America). Ligands with the lowest protein-ligand interaction scores represented in silico inhibitors of GAPDHS. Three pockets were selected for the initial docking analysis in mouse Gapdhs based on a SYBYL® Site ID analysis of the surface of the mouse Gapdhs homology model. The Site ID algorithm solvates the protein structure and then locates regions where the solvent atoms tend to cluster in a pocket. Pocket 1 covered the region of the active site where the G3P substrate and the nicotinamide moiety of the cofactor bind. This first pocket comprises the following residues: in mouse Gapdhs, R115, I116, L119, T223, S253, C254, T255, H281, S282, Y283, T284, A285, T313, G314, P340, N418, E419, Y422, and S423; in human GAPDHS, R85, I86, G87, L89, S193, S223, C224, T225, H251, S252, Y253, T254, A255, T283, G284, P310, N388, E389, Y392, and S393; and in human GAPDH, R12, 113, L16, S121, S150, C151, T152, H178, A179, 1180, T181, A182, T210, G211, A237, N315, E316, Y319, and S320. Pocket 2 covered the region of the active site where the adenine moiety of the cofactor binds. This second pocket comprises the following residues: in mouse Gapdhs, N111, G112, F113, G114, N135, D136, P137, F138, C180, K181, D182, P183, E198, C199, T200, V202, and Y203; in human GAPDHS, N81, G82, F83, G84, N105, D106, P107, F108, C150, K151, 152, P153, E168, 169, T170, V172, Y173; and in human GAPDH, N8, G9, F10, G11, N33, D34, P35, F36, E78, R79, D80, P81, E96, S97, T98, V100, and F101. Pocket 3 covered the inorganic phosphate-binding site, which is required for the addition of a second phosphate group to the G3P substrate during catalysis. This third pocket comprises the following residues: in mouse Gapdhs, T284, A285, T286, Q287, K288, S294, D297, R299, G300, G301, I309, P310, S311, S312, A334, and R336; in human GAPDHS, T254, A255, T256, Q257, K258, S264, A267, R269, D270, G271, I279, P280, A281, S282, A304, R306; and in human GAPDH, T181, A182, T183, Q184, K185, S191, L194, R196, D197, G198, 1206, P207, A208, S209, A231, R233. Additional docking studies were conducted for a smaller pocket that is more closely confined to the substrate, defined hereinabove as Small Pocket 1. This pocket comprises the following residues in mouse Gapdhs, S253, C254, T255, T313, T314; in human GAPDHS, S223, C224, T225, T283, G284; and in human GAPDH, S150, C151, T152, T210, G211.

In all docking studies, interactions between ligand and protein are not limited to just the pocket or site residues, but can also include neighboring protein residues. Docking studies were completed for each of the pockets, comparing human GAPDHS and human GAPDH to select ligands that exhibit specificity between GAPDH and GAPDHS isoforms.

Docking via the FlexX algorithm involves a plant-and-grow methodology as follows. First, the ligand is fragmented at natural points. Second, the ligand fragments are placed in the active site optimizing the protein-ligand interaction scores. And finally, the complete ligand is reconstructed from the fragments. This is a fast algorithm that takes into account the critical conformational flexibility of the ligand.

The FlexX protein-ligand interaction score incorporates hydrogen-bond, salt bridge, and non-polar terms along with additional entropic and enthalpic terms accounting for the number of rotatable bonds in the ligand and the buried surface area.

The initial library used in docking was Volume 10 (Jun. 1, 2003) of the Ryan Scientific, Inc. (Isle of Palms, S.C., United States of America) high throughput screening database that included more than 303,000 compounds. This library is Ryan Scientific's composite database including compounds offered by many of their worldwide principals, all in just one database.

From the library docking, ligands were identified that bind to pockets 1, 2, 3, and Small Pocket 1 of GAPDHS. Ligands were identified that should preferentially bind GAPDHS in the active site, compete with the cofactor and/or substrate, and therefore act as specific inhibitors of GAPDHS.

### Pharmacophore Model for Pocket 1

Table 6 highlights the hydrogen bond donors and acceptors in human GAPDHS (GENBANK® Accession No. 014556) in the region of Pocket 1 that are involved in binding the inhibitors. The structures of fifteen representative inhibitors that bind to Pocket 1 (LT_{—}01147981, M2H_{—}923, T0508_{—}6550, T0506_{—}8000, LT 00439522, LT_{—}00096886, RJF 01571, HTS 08814, and LT_{—}00232244, T0511_{—}4909, T05017749) or Small Pocket 1 (T0507_{—}5482, T0504_{—}1251, LT_{—}00249157, 11K_{—}064) are shown in FIGS. 6A-6L.

TABLE 6
Hydrogen Bond Donors and Acceptors in the Region of
Pocket 1 and Small Pocket 1

| Residue | | | | Residue involved in |
|---|---|---|---|---|
| in | | Side Chain | Donor or | cofactor or |
| GAPDHS | Atom | or Backbone | Acceptor | substrate binding? |
| Arg85 | N | BB | D | Yes |
| Ile86 | N | BB | D | Yes |
| Gly87 | N | BB | D | |
| Ser223 | OG | SC | D | Yes |
| Cys224 | N | BB | D | Yes |
| Thr225 | OG | SC | D | Yes |
| Ser252 | OG | SC | D/A | |
| Tyr253 | O | BB | A | |
| Tyr253 | OH | SC | D | |
| Thr254 | OG | SC | A | Yes |
| Ala255 | N | BB | D | Yes |
| Gly284 | N | BB | D | |
| Pro310 | O | BB | A | |
| Asn388 | ND2 | SC | D | Yes |
| Asn388 | O | BB | A | Yes |
| Glu389 | OE2 | SC | A | |

### Inhibitors

Table 7 presents inhibitors that show preferential binding to GAPDHS in Pocket 1 and Small Pocket 1. FIGS. 6A-60 show the structures of these compounds. These compounds, which compete for binding with the substrate and/or the nicotinamide moiety of the NAD cofactor, were tested for inhibition of recombinant human GAPDH and a truncated form of human GAPDHS (see Example 7).

TABLE 7
Inhibitors that Bind to Pocket 1 or Small Pocket 1

| | | Formula, | | | | | | |
|---|---|---|---|---|---|---|---|---|
| | | Mr, | | | | | | |
| | | physical | | | | | H-bonds | H-bonds |
| | | characteristics, | Group of | Score | IC₅₀ | | Sperm | muscle |
| | Compound | solubility (+/−) | compounds | discrimination | GAPDHS | IC₅₀ GAPDH | GAPDHS | GAPDH |
| Small Pocket 1 | T0507_5482 | C₇H₅N₂OF₃, | 2,2,2-Trifluoro-1-(3vinyl-3H- | −8.8 | >1 mM | >1 mM | Ser223 | Gly211 |
| | | Mr = 190.13, | imidazol-4-yl)-ethanone | | | | Thr225 | |
| | | white, | | | | | | |
| | | DMSO+ | | | | | | |
| | | H₂O+ | | | | | | |
| | T0504_1251 | C₈H₈N₂O, | (1H-Benzoimidazol-2-yl)- | −5.5 | >3 mM | not | Ser223 | Gly211 |
| | | Mr = 148.16, | methanol | | | tested | Gly284 | Thr210 |
| | | Yellow, | | | | | | |
| | | DMSO+ | | | | | | |
| | | H₂O+ | | | | | | |
| | LT_00249157 | C₅H₇N₅O₃, | 3-methyl-5-oxo-pyrazole-1- | −3.2 | 0.1 mM | 1 mM | Gly284 | Gly211 |
| | | Mr = 185.14, | nitro-carboxamidine | | | | Ser223 | Thr210 |
| | | Pink, | | | | | | Thr152 |
| | | DMSO+/− | | | | | | Cys151 |
| | | H₂O+ | | | | | | Ser150 |
| | 11K_064 | C₈H₃Cl₃N₂S₂, | 2,4-dichloro-N-(4chloro-5H- | −5.9 | 0.5 mM | 0.1 mM | Ser223 | Gly211 |
| | | Mr = 297.61 | 1,2,3-dithiazol-5- | | | | Gly284 | |
| | | Yellow | yliden)aniline | | | | | |
| | | DMSO+ | | | | | | |
| | | H₂O+ | | | | | | |
| Pocket 1 | RJF 01571 | C₁₄H₁₆N₂O₄, | 2-[(Oxo-4-propyl-2H- | −8.8 | 1 mM | not | Ser252 | Ile180 |
| | | Mr = 276.89, | chromen-7-y)loxy] | | | tested | Asn388 | Ala237 |
| | | White, | ethanohydrazide | | | | Pro310 | Asn315 |
| | | DMSO+ | | | | | Tyr253 | Glu316 |
| | | H₂O+ | | | | | Glu389 | Cys151 |
| | | | | | | | Ala255 | |
| | | | | | | | Ile86 | |
| | | | | | | | Gly87 | |
| | HTS 08814 | C₁₉H₁₄N₆O₂, | 6-methyl-8-{[5-(2-naphthyl)- | −9.3 | >2 mM | not | Ala255 | Ala182 |
| | | Mr = 358.35, | 1,3,5-oxadiazol-2- | | | tested | Ile86 | Arg12 |
| | | Cream, | yl]methoxy}[1,2,4]triazolo[4,3- | | | | Gly87 | Asp315 |
| | | DMSO+/− | b]pyridazine | | | | | |
| | | H₂O+ | | | | | | |
| | LT_00232244 | C₂₇H₂₈N₄O₃, | 3-{3-(4-Dimethylamino- | −14.3 | >1 mM | 0.35 mM | Ala255 | Ser121 |
| | | Mr = 456.54, | phenyl)-3-[(pyridine-4- | | | | Thr254 | Cys151 |
| | | Orange, | ylmethyl)-amino]-propionyl}- | | | | Asn388 | |
| | | DMSO+/− | 4-hydroxy-1-methyl-1H- | | | | | |
| | | H₂O+/− | quinolin-2-one | | | | | |
| | T0511_4909 | C₂₃H₂₅N₃O₆S | 3-(1H-Indol-3-yl)-propionic | −8.4 | 0.5 mM | >1 mM | Tyr253 | Ala182 |
| | | Mr = 471.54, | acid [4-(morpholine-4- | | | | Ala255 | ASn315 |
| | | Pink | sulfonyl)-phenylcaramoyl]- | | | | Asn388 | Gly14 |
| | | DMSO+ | methyl ester | | | | | Ile13 |
| | | H₂O+/− | | | | | | |
| | T0501_7749 | C₂₅H₂₁N₅O₅S | 2-[2-Amino-3-(toluene-4- | −9.1 | 0.1 mM | >1 mM | Ala255 | Ala182 |
| | | Mr = 503.54 | sulfonyl)-pyrrolo[2,3- | | | | Tyr253 | Thr181 |
| | | Yellow | b]quinoxalin-1-yl]-1-(4-nitro- | | | | Pro310 | |
| | | DMSO+ | phenyl)-ethanol | | | | | |
| | | H₂O+ | | | | | | |

### Pharmacophore Model for Pocket 2

Table 8 highlights the hydrogen bond acceptors in human GAPDHS (GENBANK® Accession No. 014556) in the region of Pocket 2 that are involved in binding the inhibitors.

TABLE 8
Hydrogen Bond Donors and Acceptors In the Region of Pocket 2

| | | | | Residue |
|---|---|---|---|---|
| | | | | involved in |
| Residue in | | Side Chain | Donor or | cofactor |
| GAPDH2 | Atom | or Backbone | Acceptor | binding? |
| Asn81 | OD1 | SC | A | |
| Asn81 | ND2 | SC | D | |
| Phe83 | N | BB | D | Yes |
| Gly84 | N | BB | D | Yes |
| Asn105 | ND2 | SC | D | Yes |
| Asp106 | OD1 & | SC | A | Yes |
| | OD2 | | | |
| Phe108 | N | BB | D | Yes |
| Cys150 | O | BB | A | Yes |
| Lys151 | O | BB | A | Yes |
| Glu152 | OD1 & | SC | A | |
| | OD2 | | | |
| Ser169 | O | BB | A | Yes |
| Thr170 | O | BB | A | Yes |
| Tyr173 | OH | SC | A/D | |

### Inhibitors

Table 9 presents inhibitors which show preferential binding to GAPDHS in Pocket 2. FIGS. 7A-71 show the structures of these compounds. These compounds compete for binding with the adenine moiety of the NAD cofactor. Several of these compounds were tested for inhibition of recombinant human GAPDH and a truncated form of human GAPDHS (see Example 7).

TABLE 9
Inhibitors that Bind to Pocket 2

| | | Formula, | | | | | | | |
|---|---|---|---|---|---|---|---|---|---|
| | | Mr, | | | | | | | |
| | | physical | | | | | | H-bonds | H-bonds |
| | | characteristics, | Group of | Score | IC₅₀ | | IC₅₀ | Sperm | muscle |
| | Compound | solubility (+/−) | compounds | discrimination | GAPDHS | | GAPDH | GAPDHS | GAPDH |
| Pocket 2 | LT_00444480 | C₁₇H₁₇N₇O₇, | (1,3-Dimethyl-2,6-dioxo- | −9.1 | 0.15 | mM | 0.15 mM | Tyr173 | Arg79 |
| | | Mr = 431.36 | 1,2,3,6-tetrahydro-purin- | | | | | Lys151 | Asp80 |
| | | Yellow, | 9-yl)-acetic acid (4- | | | | | Asn81 | |
| | | DMSO+/− | hydroxy-3-methoxy-5- | | | | | Asn105 | |
| | | H₂O+ | nitro-benzylidene)- | | | | | Asp106 | |
| | | | hydrazide | | | | | | |
| | T0501_7933 | C₂₃H₂₀N₆, | 4-(1-Benzyl-IH-indol- | −15 | 0.01-0.025 | mM | >0.5 mM | Tyr173 | Asp80 |
| | | Mr = 380.45, | 3y1)-3,4-dihydro- | | | | | Asn81 | Arg79 |
| | | White, | benzo[4,5]imidazo[1,2- | | | | | Asn105 | |
| | | DMSO+/− | a][1,3,5]triazin-2-ylamine | | | | | | |
| | | H₂O+/− | | | | | | | |
| | LT_00729760 | C₁₄H₁₇N₅O₅S₁, | 3-(1H-Imidazolo-4-yl)-2- | −11.5 | 2 | mM | not | Asn81 | Asp34 |
| | | Mr = 383.45, | [3-(4-sulfamoyl-benzyl)- | | | | tested | Tyr173 | Arg79 |
| | | White, | thioureido]-propionic | | | | | Cys150 | Thr98 |
| | | DMSO+/− | acid | | | | | Asn105 | |
| | | H₂O+ | | | | | | Asp106 | |
| | | | | | | | | Gly84 | |
| | | | | | | | | Ser169 | |
| | LT_00379844 | C₁₈H₁₂N₂O₇, | 5-[2-(1,3-Dioxo-1,3- | −10.8 | >1 | mM | not | Tyr173 | Arg79 |
| | | Mr = 368.29, | dihydro-isoindol-2-yl)- | | | | tested | Lys151 | Asp80 |
| | | White, | acetylamino]-isophthalic | | | | | Asp106 | Glu78 |
| | | DMSO+ | acid | | | | | Thr170 | |
| | | H₂O+ | | | | | | | |
| | LT00105955 | C₁₆H₁₅N₇O₄ | [5-(4-Methoxy- | −10.7 | >1 | mM | >1 mM | Tyr173 | Arg79 |
| | | Mr = 369.34 | phenylamino)-2-oxa- | | | | | Phe83 | |
| | | Yellow | 1,3,4,6,7,8a-hexaaza-as- | | | | | Asn81 | |
| | | DMSO+ | indacen-8-yl]-acetic acid | | | | | Asn105 | |
| | | H₂O+/− | ethyl ester | | | | | | |
| | T0506_9350 | C₂₀H₂₅N₅O₆S | 4-Amino-N-(4-methoxy- | −9 | 0.5 | mM | >1 mM | Phe108 | Gly11 |
| | | Mr = 463.52 | phenyl)-3-nitro- | | | | | Tyr173 | Phe10 |
| | | Orange | benzenesulfonamide- | | | | | Lys151 | Thr98 |
| | | DMSO+ | cyclohexyl-urea | | | | | Asp106 | Asp34 |
| | | H₂O+/− | | | | | | Asn81 | |
| | | | | | | | | Asn105 | |
| | T0507_0666 | C₂₅H₂₆CIN₅O₅S, | N-[1-(3- | −6.1 | 1 | mM | not | Lys151 | Thr98 |
| | | Mr = 544.03, | Isopropenylphenyl)-1- | | | | tested | Tyr173 | Asp34 |
| | | Orange, | methylethyl]-2[N-(4- | | | | | Asp106 | |
| | | DMSO+ | chloro-phenyl)-3-nitro- | | | | | Phe108 | |
| | | H₂O+/− | benzenesulfonamide]- | | | | | Asn81 | |
| | | | hydrazinecarboxamide | | | | | Asn105 | |
| | T0506_1274 | C₂₀H₁₆N₅O₅SF₃, | 2-[4-(anilinosulfonyl)-2- | −9.8 | 1.75 | mM | not | Asn81 | Thr98 |
| | | Mr = 495.44, | nitrophenyl]-N-[3- | | | | tested | Tyr173 | Asp34 |
| | | Yellow, | (trifluoromethyl)phenyl] | | | | | Asp106 | Phe10 |
| | | DMSO+ | hydrazinecarboxamide | | | | | Lys151 | Arg79 |
| | | H₂O+/− | | | | | | Asn105 | |
| | | | | | | | | Phe108 | |
| | LT_01178124 | C₂₄H₂₃N₅O₇, | N-(3,4-Dimethyl-phenyl)- | not | not soluble | | not | Tyr173 | Asp34 |
| | | Mr = 493.47, | 2-{4-[(2,4-dinitro-phenyl)- | determined | | | tested | Asp106 | |
| | | Red, | hydrazonomethyl]-2- | | | | | Gly84 | |
| | | DMSO− | methoxy}-acetamide | | | | | Phe83 | |
| | | H₂O− | | | | | | | |
| | LT_00587256 | C₂₁H₁₇N₃O₃Cl₂, | N-(3,4-Dichloro-phenyl)- | not | 5 | mM | not | Tyr173 | [—] |
| | | Mr = 429.28 | 3-[(3-hydroxy- | determined | | | tested | Lys151 | |
| | | Yellow, | naphthalene-2- | | | | | | |
| | | DMSO+ | carbonyl)-hyrazono]- | | | | | | |
| | | H₂O+/− | butyramide | | | | | | |
| | LT_00237350 | C₁₉H₁₄N₈O₆, | [2-(4-Aminofurazan-3- | −4.7 | 2 | mM | not | Tyr173 | Glu78 |
| | | Mr = 450.35, | yl)-benzoimidazol-1-yl]- | | | | tested | Lys151 | |
| | | Yellow, | acetic acid [1-(6-nitro- | | | | | Asp106 | |
| | | DMSO+ | benzo[1,3]dioxol-5-yl)- | | | | | Asn81 | |
| | | H₂O− | meth-(Z)ylidene]- | | | | | Asn105 | |
| | | | hydrazide | | | | | Gly84 | |

### Pharmacophore Model for Pocket 3

Table 10 highlights the hydrogen bond acceptors in human GAPDHS (GENBANK® Accession No. 014556) in the region of Pocket 3 that are involved in binding the inhibitors.

TABLE 10
Hydrogen Bond Donors and Acceptors In the Region of Pocket 3

| Residue | | | | |
|---|---|---|---|---|
| In | | Side Chain | Donor or | Residue involved in |
| GAPDHS | Atom | or Backbone | Acceptor | substrate binding? |
| Thr254 | OG | SC | D | Yes |
| Asp270 | OD1 | SC | A | |
| Ser282 | N | BB | D | |
| Ser282 | O | BB | A | |
| Arg306 | NH2 & | SC | D | Yes |
| | NH1 | | | |

### Inhibitors

Table 11 presents inhibitors which show preferential binding to GAPDHS in Pocket 3. FIGS. 8A-8C show the structures of these compounds. These compounds were tested for inhibition of recombinant human GAPDH and a truncated form of human GAPDHS (see Example 7).

TABLE 11
Inhibitors that Bind to Pocket 3

| | | Formula, | | | | | | |
|---|---|---|---|---|---|---|---|---|
| | | Mr, | | | | | | |
| | | physical | | | | | H-bonds | H-bonds |
| | | characteristics, | Group of | Score | IC₅₀ | IC₅₀ | Sperm | muscle |
| | Compound | solubility (+/−) | compounds | discrimination | GAPDHS | GAPDH | GAPDHS | GAPDH |
| Pocket 3 | T0515_4928 | C₁₂H₇NO₃, | 2-Nitro-dibenzofuran | −17.6 | 0.1 mM | 1 mM | Thr254 | Thr181 |
| | | Mr = 213.19, | | | | | Arg306 | Ala182 |
| | | yellow, | | | | | Ser282 | Thr183 |
| | | DMSO+/− | | | | | | |
| | | H₂O− | | | | | | |
| | T0512_4753 | C₈H₁₀N₂O₃, | (2-Methoxy-5-nitro- | −16.9 | >1 mM | >1 mM | Thr254 | Arg196 |
| | | Mr = 182.18, | phenyl)-methyl-amine | | | | Arg306 | Ser209 |
| | | Red, | | | | | Ser282 | |
| | | DMSO+ | | | | | | |
| | | H₂O+ | | | | | | |
| | LT_00050115 | C₇H₁₀N₂O₂, | N-(Acryloylamino- | −14.5 | >1 mM | >1 mM | Thr254 | Ser209 |
| | | Mr = 154.17, | methyl)-acrylamide | | | | Arg306 | Arg233 |
| | | White, | | | | | Ser282 | |
| | | DMSO+ | | | | | Asp270 | |
| | | H₂O+ | | | | | | |

### Discussion of Examples 1-6

Although the spermatogenic and somatic GAPDH isoforms possess high sequence identity particularly in the catalytic domain, there are sufficient differences in sequence to have an impact on the enzyme structure and particularly the conformation of the substrate-binding pocket. The differences in eight highly conserved residues in GAPDH in the spermatogenic isoforms near the binding pocket might be responsible for differences in the responses and preferential binding of (S)-3-chlorolacetaldehyde metabolites to the spermatogenic and somatic isoforms.

The different sizes and steric and chemical properties of the residues in the spermatogenic isoform cause protein loops around the substrate-binding pocket to adopt different conformations, and create a difference in the molecular accessible surface area for binding. The inhibitor buries a larger amount of the accessible surface on binding to GAPDHS and Gapdhs than on binding to GAPDH. This means that there is larger surface area contact between the enzyme and ligand in the spermatogenic isoforms.

GAPDHS and Gapdhs molecular models are very similar in structure with less than a 1 angstrom RMSD. This suggests that the biological activities of the mouse and human spermatogenic GAPDH should be similar. The models disclosed herein are believed to be accurate homology models since they are based on the somatic structure with which they share at least 60% sequence identity.

The docking studies of glyceraldehyde 3-phosphate and S-3-chloroacetaldehyde disclosed herein illustrate that GAPDHS appears to have a greater surface accessible area for ligand docking, and that when the ligand binds it covers more of this accessible surface. The ligand must have a more extended conformation on binding that covers more of the surface accessible area in the spermatogenic isoform than in the somatic one. The eight residues surrounding the substrate-binding pocket that are identical in GAPDHS and Gapdhs, but replace conserved residues in GAPDH, also have a much greater surface accessible area in GAPDHS/Gapdhs. The difference in the access the ligand has to the substrate-binding pocket in GAPDHS/Gapdhs is due largely to these eight residues.

Additional docking studies of a large database of compounds in higher resolution models of GAPDHS and Gapdhs (Example 6) identified potential inhibitors that bind to three distinct pockets (Pockets 1, 2, and 3) and to Small Pocket 1. Inhibitors were identified that bound to three of the amino acids designated in the initial homology models (Examples 14) as distinctly different between the sperm and somatic enzymes (Table 2). These amino acids are Y173, S252 and Y253 in human GAPDHS or Y203, S282, Y283 in mouse Gapdhs. Residue Y173/Y203 appears particularly significant for binding potential inhibitors that would selectively inhibit cofactor binding to Pocket 2 of the sperm isozyme. See FIG. 9. The corresponding residue in somatic GAPDH (F101 human, F100 mouse) does not form hydrogen bonds with these inhibitors, indicating that these compounds should not inhibit the somatic enzyme.

### Example 7

### In Vitro Testing of Gapdhs Modulators on Recombinant Enzymes

Recombinant mouse Gapdh and a truncated Gapdhs (tGapdhs; containing amino acids 106-438 of mouse Gapdhs) were initially expressed as Glutathione-5-transferase (GST) fusion proteins to compare activities and inhibition. The truncated form is 71% identical to Gapdh and lacks only the proline-rich N-terminus that anchors Gapdhs to the fibrous sheath in the sperm flagellum. Soluble fusion proteins were purified and GST was removed by thrombin cleavage. Western blots confirmed the size (36 kDa) and specific immunoreactivity of each isozyme. Activities of the recombinant enzymes were determined with a standard assay measuring the increase in absorption at 340 nm resulting from reduction of the NAD cofactor (Velick, 1955). The activities of recombinant Gapdhs (28.5±0.3 units/mg) and rabbit muscle Gapdh (27.7±0.3 units/mg) were similar and ˜15% lower than the activity of recombinant tGapdhs (33.6±0.2). These measurements were done with the standard assay adapted to a 96-well format. This assay is suitable for high-throughput screening.

Adenine nucleotides that act as competitive inhibitors of NAD cofactor binding (ATP, ADP, AMP, cyclic AMP) caused dose-dependent inhibition of both Gapdhs and tGapdhs. UTP and adenosine did not inhibit either of the recombinant enzymes. Inhibition of Gapdhs was more pronounced than inhibition of tGapdhs with each of these adenine nucleotides at concentrations from 1-10 mM. The calculated Ki values for ATP, ADP, AMP, and cAMP were 6.48, 3.97, 3.13, and 3.33 mM for tGapdhs and 1.27, 1.79, 1.61, and 1.5 mM for Gapdh. See FIGS. 11A-11D. These studies provide the first evidence that the sperm and somatic Gapdh isozymes can be differentially inhibited by compounds that interfere with NAD cofactor binding.

In subsequent studies to test inhibitors identified in computer docking studies, recombinant mouse Gapdh and the truncated form of Gapdhs (tGapdhs, amino acids 106-438) were expressed in DS112 gapA-deficient E. coli strain K-12 (available from the Yale University E. coli Genetics Stock Center, New Haven, Conn., United States of America). The truncated Gapdhs lacks the proline-rich N-terminus, but contains all the amino acids required for enzyme activity. The recombinant proteins were purified and activities were determined with a standard enzyme assay (Schmalhausen et al., 1997). Using this assay, the dehydrogenase activity of Gapdh and tGapdhs was monitored at 340 nm by measuring the accumulation of NADH.

The reaction mixture (200 μl) contained 50 mM glycine, 50 mM potassium phosphate, 5 mM EDTA (pH 8.9), 1 μg of recombinant enzyme (Gapdh or tGapdhs), 0.5 mM NAD, and 0.5 mM glyceraldehyde-3-phosphate. Activities of Gapdh and tGapdh were compared in the presence of various concentrations of inhibitors. Compounds identified as potential inhibitors in modeling studies as described herein were obtained from Ryan Scientific (Mt. Pleasant, S.C., United States of America). Stocks for each compound were prepared by dissolving 5 mg in 50 μl of DMSO. These stocks were divided into 10 μl aliquots and frozen at −20° C.

To monitor inhibition, each compound (1 μM to 3 mM) was added to the above reaction mixture (without NAD and glyceraldehyde-3-phosphate) and incubated for 1 hour at 4° C. After the 1 hour incubation period, NAD and glyceraldehyde-3-phosphate were added to start the reaction. In each assay, controls containing equal amounts of DMSO were included to determine values for 100% activity. The IC₅₀ for each compound was calculated as the concentration required to reduce dehydrogenase activity to 50% of the activity of control samples. The IC₅₀ values determined in these studies are shown in Tables 7, 9, and 11. Lower IC₅₀ values were observed for Gapdhs compared to Gapdh with several compounds, indicating that these compounds showed the selective inhibition of Gapdhs predicted by computer docking. Compounds that showed greater inhibition of Gapdhs than Gapdh include LT_{—}00249157 (Small Pocket 1), T0511_{—}4909 (Pocket 1), T0501_{—}7749 (Pocket 1), T0501_{—}7933 (Pocket 2), T0506_{—}9350 (Pocket 2), and T0515_{—}4928 (Pocket 3). An example of selective inhibition is shown in FIG. 12.

### Example 8

### Generation of Gapdhs^{-/-} Knockout Mice

A genomic clone comprising the Gapdhs gene was isolated from a P1 phage library of 129/OlaHsd mouse genomic DNA (Incyte Corp., Palo Alto, Calif., United States of America). Following digestion with Tth111 I and Ssp I, the DNA restriction fragment beginning in exon 1 and ending in intron 4 (nucleotides 1887-7193 of SEQ ID NO: 22) was cloned into the Srf I site of the cloning vector pUCBM21/KO (Dix et al., 1996). This fragment, along with thymidine kinase (tk) and neomycin resistance (neo) genes, was ligated into the Not I-Xho I site of pMC1TKbpA (gift of Dr. Yuji Mishina, National Institute of Environmental Health Sciences, Research Triangle Park, N.C., United States of America), which contains another tk cassette. The Gapdhs DNA fragment beginning in exon 6 and ending in exon 9 (nt 8108-9181 of SEQ ID NO: 22) was amplified by PCR using the P1 clone as template and then ligated into the Spe I-Xba I sites of pMC1TKbpA to produce the gene-targeting construct pL3KaSTK2 (see FIG. 10A). Transfection of pL3KaSTK2 DNA, screening of targeted TC-1 embryonic stem (ES) cells (gift of Dr. Philip Leder, Harvard Medical School, Boston, Mass., United States of America), and blastocyst injections were performed as in Dix et al., 1996. The PCR primers used to detect homologous recombination were forward 5′-AGCGTTGGCTACCCGTGATA-3′ (SEQ ID NO: 23) and reverse 5′-CGTGATAGCCGAGTMGMGCAGG-3′ (SEQ ID NO: 24), corresponding to sequences in the neo gene and exon 9, respectively. Genotypes were confirmed by Southern blotting after digestion with Dra I, using a probe (nt 1080-1830 of SEQ ID NO: 22) outside the targeting construct (see FIG. 10A). Chimeric males generated from correctly targeted ES cells were mated with C57BL6/N females to obtain germline transmission. Heterozygous animals were mated to produce Gapdhs^{-/-} and Gapdhs^{+/+} males for the analysis of fertility and sperm function.

### Example 9

### Western Blotting and Immunocytochemistry

Sperm were collected from the cauda epididymis, washed in phosphate-buffered saline (PBS; 140 mM NaCl, 10 mM phosphate buffer, pH 7.4), and lysed in SDS sample buffer. Testis lysates were prepared by homogenization in lysis buffer (1 ml/testis) containing 140 mM NaCl; 0.1% Triton X-100; Complete protease inhibitor cocktail (Roche Applied Science, Indianapolis, Ind., United States of America); and 20 mM HEPES buffer, pH 7.4. Lysates corresponding to 2×10⁴ sperm or 0.5 mg testis (wet weight) were analyzed by Western blotting using anti-mouse Gapdhs antibody B1 as described in Miki et al., 2002. For the detection of Gapdhs by indirect immunofluorescence, sperm were fixed on glass slides and stained with the same anti-mouse Gapdhs antibody.

### Example 10

Gapdhs Enzyme Activity

Cauda epididymal sperm were washed with PBS, suspended in enzyme assay buffer (see Welch et al., 2000), and sonicated for 7 seconds on ice. The Gapdhs/Gapdh enzyme assay was carried out as described in Velick, 1955. To monitor Gapdhs substrate accumulation, cauda epididymal sperm were collected and incubated in M16 medium (Specialty Media, Phillipsburg, N.J., United States of America) at 37° C. in 5% CO₂ and air. Glyceraldehyde 3-phosphate was assayed as described in Racker, 1983, immediately after collection (time=0) and after incubation for 90 minutes.

### Example 11

### Electron Microscopy

Transmission and scanning electron microscopy were performed as described in Miki et al., 2002, except that sperm analyzed by scanning electron microscopy were treated with 1% Triton X-100 in PBS for 15 minutes at room temperature before fixation with 5% glutaraldehyde in 0.2 M sodium cacodylate buffer.

### Example 12

### Fertility and Sperm Motility

To determine fertility, individual Gapdhs^{-/-} males were mated continuously with two wild type females for one month, followed by a second month with two different females. Individual Gapdhs^{-/-} females were mated with one wild type male for two months. To assess motility, sperm were collected from the cauda epididymis of Gapdhs^{-/-} and wild type mice in M16 medium (Specialty Media, Phillipsburg, N.J., United States of America) and incubated at 37° C. with 5% CO₂ in humidified air. Parameters of sperm motility were assessed immediately after collection (time 0) and after 1 hour using an HTM-IVOS motility analyzer with software version 12 (Hamilton Thorne Research, Beverly, Mass., United States of America) for computer assisted-sperm analysis (CASA). The results of these analyses are presented in Table 12.

TABLE 12
Sperm Motility in Gapdhs Knockout Mice

| | | | | | | | | | | | | |
|---|---|---|---|---|---|---|---|---|---|---|---|---|
| | | | | Trks | | | | | | | | |
| Mouse | Type | % Motile | % Prog. | Prog. | N | VSL | VAP | VCL | ALH | BCF | STR | LIN |

Time = 0

| | | | | | | | | | | | | |
|---|---|---|---|---|---|---|---|---|---|---|---|---|
| 2 | WT | 32.0 | 19.7 | 61.7 | 188 | 68.5 | 97.2 | 228.7 | 14.5 | 37.3 | 64.7 | 30.1 |
| 3 | | 36.6 | 19.6 | 53.4 | 511 | 56.7 | 95.2 | 226.4 | 16.2 | 38.6 | 53.4 | 23.3 |
| 4 | | 45.0 | 30.3 | 67.3 | 392 | 63.9 | 94.1 | 214.4 | 14.9 | 37.4 | 62.0 | 28.4 |
| Mean | | 37.9 | 23.2 | 60.8 | | 63.0 | 95.5 | 223.2 | 15.2 | 37.8 | 60.0 | 27.3 |
| Std Dev | | 6.6 | 6.2 | 7.0 | | 6.0 | 1.6 | 7.7 | 0.9 | 0.7 | 5.9 | 3.5 |
| Std Err | | 3.8 | 3.6 | 4.0 | | 3.4 | 0.9 | 4.4 | 0.5 | 0.4 | 3.4 | 2.0 |
| 1 | KO | 46.1 | 0.0 | 0.0 | 205 | 8.1 | 53.7 | 112.2 | 10.1 | 51.9 | 15.8 | 7.8 |
| 2 | | 36.9 | 0.0 | 0.0 | 187 | 8.3 | 57.7 | 118.5 | 10.3 | 53.1 | 15.1 | 7.7 |
| 3 | | 39.8 | 0.1 | 0.3 | 334 | 9.9 | 55.2 | 115.3 | 9.6 | 50.5 | 18.9 | 9.2 |
| 4 | | 24.0 | 0.0 | 0.0 | 188 | 6.6 | 36.9 | 86.1 | 6.6 | 55.6 | 20.4 | 9.0 |
| Mean | | 36.7 | 0.0 | 0.1 | | 8.2 | 50.9 | 108.0 | 9.1 | 52.8 | 17.5 | 8.4 |
| Std Dev | | 9.3 | 0.1 | 0.2 | | 1.4 | 9.5 | 14.8 | 1.7 | 2.1 | 2.5 | 0.8 |
| Std Err | | 4.7 | 0.0 | 0.1 | | 0.7 | 4.7 | 7.4 | 0.9 | 1.1 | 1.3 | 0:4 |
| p = * | | 0.8601 | 0.0006 | <0.0001 | | <0.0001 | 0.0005 | <0.0001 | 0.0270 | <0.0001 | <0.0001 | 0.0001 |

Time = 1 hr

| | | | | | | | | | | | | |
|---|---|---|---|---|---|---|---|---|---|---|---|---|
| 2 | WT | 54.3 | 28.9 | 53.2 | 378 | 54.2 | 82.7 | 187.3 | 12.8 | 39.7 | 59.8 | 28.1 |
| 3 | | 41.0 | 21.7 | 53.0 | 498 | 56.2 | 89.6 | 220.1 | 14.9 | 40.3 | 56.2 | 24.5 |
| 4 | | 42.4 | 21.4 | 50.5 | 319 | 51.8 | 86.7 | 210.8 | 14.3 | 40.6 | 54.4 | 23.9 |
| Mean | | 45.9 | 24.0 | 52.2 | | 54.1 | 86.3 | 206.1 | 14.0 | 40.2 | 56.8 | 25.5 |
| Std Dev | | 7.3 | 4.2 | 1.5 | | 2.2 | 3.4 | 16.9 | 1.1 | 0.4 | 2.7 | 2.3 |
| Std Err | | 4.2 | 2.4 | 0.9 | | 1.2 | 2.0 | 9.8 | 0.6 | 0.3 | 1.6 | 1.3 |
| 1 | KO | 13.9 | 0.0 | 0.0 | 29 | 5.1 | 36.1 | 77.4 | 4.9 | 56.0 | 16.6 | 7.5 |
| 2 | | 23.0 | 0.0 | 0.0 | 73 | 5.5 | 51.0 | 103.5 | 8.1 | 56.8 | 12.5 | 6.1 |
| 3 | | 41.9 | 0.0 | 0.0 | 174 | 6.6 | 56.3 | 107.6 | 9.0 | 55.0 | 12.5 | 6.7 |
| 4 | | 25.4 | 0.0 | 0.0 | 92 | 6.2 | 37.8 | 88.7 | 6.6 | 54.3 | 19.4 | 7.7 |
| Mean | | 26.1 | 0.0 | 0.0 | | 5.9 | 45.3 | 94.3 | 7.2 | 55.5 | 15.2 | 7.0 |
| Std Dev | | 11.7 | 0.0 | 0.0 | | 0.7 | 9.9 | 13.9 | 1.8 | 1.1 | 3.4 | 0.7 |
| Std Err | | 5.8 | 0.0 | 0.0 | | 0.3 | 4.9 | 6.9 | 0.9 | 0.6 | 1.7 | 0.4 |
| p = | | 0.0509 | <0.0001 | <0.0001 | | <0.0001 | 0.0011 | 0.0002 | 0.0022 | <0.0001 | <0.0001 | <0.0001 |

* Statistical analyses of CASA parameters for sperm from wild type (WT) and Gapdhs^{-/-} (KO) mice were conducted using the General Linear Models procedure in SAS (SAS Institute, Cary, North Carolina, United States of America).

### Example 13

### Sperm ATP Levels

Following incubation in M16 medium as described above, sperm were centrifuged at 1,000×g for 3 minutes. Excess medium was removed and sperm in the remaining 50 μl were re-suspended, transferred to a tube containing 450 μl of boiling extraction buffer (4 mM EDTA; 0.1 M Tris-HCl, pH 7.8), and incubated at 100° C. for 2 minutes. The supernatant was collected following centrifugation at 20,000×g for 5 minutes and diluted 10-fold with water. ATP was measured in duplicate 50 μl aliquots of the supernatant using a luciferase bioluminescence assay according to the manufacturer's protocol (ATP Bioluminescence Assay kit CLS II; Roche Applied Science, Indianapolis, Ind., United States of America).

### Example 14

### Oxygen Consumption

Sperm were collected from the cauda epididymis and vas deferens in glucose-free M2 medium. Oxygen consumption of the sperm suspension was determined using an oxygen probe (YSI 5531, YSI Inc., Yellow Springs, Ohio, United States of America) calibrated to air-saturated medium. Samples containing 4-6×10⁷ sperm in glucose-free M2 medium were stirred vigorously in the reaction chamber (1.8 ml) at room temperature. The reaction was terminated by adding 1 mM KCN, and the baseline was obtained by additional monitoring. Consumed oxygen was calculated using the solubility factor of 0.237 mmole/ml.

### Discussion of Examples 8-14

A knockout mouse line was generated that comprised a targeted disruption of the Gapdhs gene (Miki et al., 2004). This was accomplished by deleting exon 5 and part of exon 6 of the mouse Gapdhs gene (see FIG. 10A), a disruption that prevents expression of the catalytic cysteine and 6 of 8 amino acids essential for NAD cofactor-binding (Welch et al., 1995). Southern blotting detected the expected mutant (8 kilobase) and wild type (˜20 kilobase) alleles in targeted embryonic stem cells, and in mice resulting from germline transmission and subsequent F1 intercrosses (FIG. 10B). GAPDHS protein was not detected in testis or sperm from Gapdhs^{-/-} males by Western blotting using antibodies that recognize peptide sequences upstream or downstream of the deletion. See FIG. 10C. The null mutation was further confirmed by the absence of Gapdhs in sperm from Gapdhs^{-/-} males in both enzyme activity (FIG. 10D) and immunocytochemical assays. Since sperm do not express the somatic GAPDH isozyme (Welch et al., 2000), elimination of Gapdhs blocks all ATP production from glycolysis. See FIG. 10E.

Gapdhs^{-/-} males were infertile, although sperm counts and testis weights did not differ from wild type males. The fertility of Gapdhs^{+/+} males and Gapdhs^{-/-} females was unaffected.

Gapdhs^{-/-} sperm have profound defects in motility, exhibiting sluggish movement without forward progression. The bend originating in the flagellar middle piece did not effectively propagate to the principal piece where Gapdhs is localized and functions in glycolysis. Increasing pyruvate concentration up to 20 mM in M16 medium did not enhance sperm motility. Table 12 shows the CASA measurements from 3 wild type (WT) and 4 Gapdhs^{-/-} mice, including statistical analyses of the results. Motility parameters measured by CASA include % motile, % progressive, % motile tracks that are progressive, straight-line velocity (VSL), velocity of the average path (VAP), curvilinear velocity (VCL), amplitude of lateral head displacement (ALH), beat cross frequency (BCF), straightness (STR), and linearity (LIN). Every parameter measured was significantly lower for the Gapdhs^{-/-} sperm compared to wild type. In samples from wild type animals, 60.8% of the motile wild type sperm were progressive (VAP>50 μm/sec and STR>50%). CASA confirmed that Gapdhs^{-/-} sperm exhibited only 0.1% progressive motility with mean straight-line velocities of 8.2 μm/sec compared to 63 μm/sec for wild type sperm (p<0.0001). Previous studies have reported that glycolysis is required for acquiring hyperactivated motility, and these studies provide evidence that progressive motility also requires ATP production by glycolysis.

A lack of progressive motility was also observed by comparing representative fields from CASA measurements of wild type and Gapdhs^{-/-} sperm. The Gapdhs^{-/-} sperm exhibited only back-and-forth motion and did not move forward.

When sperm were observed under a light microscope, the morphology of Gapdhs^{-/-} sperm was indistinguishable from wild type sperm. Minor structural differences were observed when sperm demembranated with Triton X-100 were examined by scanning electron microscopy. Gaps between some ribs of the fibrous sheath appeared wider in Gapdhs^{-/-} sperm, although the fibrous sheath assumed its normal structure surrounding the axoneme throughout the length of the principal piece of the sperm flagellum.

To understand the sluggish movement of sperm from Gapdhs^{-/-} males, ATP levels in sperm were quantified immediately after collection from the cauda epididymis. Sperm from Gapdhs^{-/-} mice had ATP levels that were only 10.4% of the levels in wild type sperm. After 4 hours, ATP levels of Gapdhs^{-/-} sperm declined to 1.9% of wild type levels, which showed no significant change over a 4 hour culture period. These results imply that most of the energy required for sperm motility is generated by glycolysis, and that the extremely low ATP levels are responsible for the lack of progressive motility and for male infertility in the Gapdhs^{-/-} males.

In general, animal cells take up pyruvate and lactate using a monocarboxylate transporter (MCT) and metabolize them via the tricarboxylic acid cycle in mitochondria to generate ATP (Halestrap & Price, 1999). MCT2 is expressed in the sperm flagellum, where it should allow the transport of pyruvate and lactate (Garcia et al., 1995). Because culture medium contains physiological concentrations of pyruvate and lactate, sperm from Gapdhs^{-/-} males should utilize these exogenous substrates for mitochondrial ATP production even if glycolysis is impaired. Sperm from Gapdhs^{-/-} and wild type mice had similar oxygen consumption levels, indicating that mitochondrial activity is indistinguishable between Gapdhs^{-/-} and wild type sperm regarding to ATP generation by the respiratory chain. These results imply that most of the energy required for sperm motility and fertility is generated by glycolysis. This conclusion is supported by a recent study demonstrating that male germ cell-specific cytocrome c (Cyt c_{T})-null mice produce functional sperm (Narisawa et al., 2002), indicating that mitochondrial oxidative phosphorylation for ATP production is not essential for sperm motility and fertility.

GAPDHS, as well as other male germ cell-specific glycolytic enzymes, have distinctive features that play a role in providing a sufficient and localized supply of ATP along the length of the flagellum. For example, GAPDHS is tightly binds to the fibrous sheath, a cytoskeletal structure in the flagellum, via its unique N-terminal sequence (Welch et al., 1992; Bunch et al., 1998). Male germ cell-specific glycolytic enzymes may also have lower Km value for substrates and/or less sensitivity to feedback regulation by ATP to increase higher local ATP concentrations required for the sperm dynein ATPase. Based on these unique features, as well as restricted expression in spermatogenic cells and sperm, GAPDHS is an excellent target for male contraceptive strategies. The infertility, lack of progressive sperm motility, and low sperm ATP levels observed in Gapdhs^{-/-} males provides direct evidence. Since GAPDHS, the ortholog of Gapdhs, is the only GAPDH isozyme in human sperm (Welch et al., 2000), inhibition of GAPDHS is likely to have similar effects on sperm motility and fertility in men.

### Example 15

### In Vitro Testing of Gapdhs Modulators on Spermatozoa

Modulators are tested in vitro for the ability to modulate a biological activity of Gapdhs in spermatozoa. The standard enzyme assay has been modified for measuring the activity of GAPDHS in mouse sperm isolated from the cauda epididymis and in ejaculated human sperm (Welch et al., 2000). Washed sperm are suspended in enzyme assay buffer containing inhibitors of enzymes downstream of GAPDH in the glycolytic pathway (Welch et al., 2000) and sonicated on ice prior to measurement of GAPDHS activity with a spectrophotometric assay (Velick, 1955). Somatic GAPDH has not been detected in mouse (Bunch et al., 1998) or human (Welch et al., 2000) sperm, and GAPDH/GAPDHS enzyme activity was absent in sperm from Gapdhs^{-/-} mice. Therefore, the standard enzyme assay measures only GAPDHS activity in sperm.

Candidate compounds are tested over a concentration range to determine if they cause dose-dependent modulation of sperm GAPDHS activity. Permeable test compounds that can cross the plasma membrane are tested by incubation with sperm maintained under conditions that support sperm viability, motility, and in vitro fertilization (Hagaman et al., 1998; Cho et al., 1998 refs given below). Candidate compounds that are not cell-permeable are tested on sperm permeabilized by sonication as described above. Since GAPDHS is tightly bound to the fibrous sheath in the sperm flagellum (Bunch et al., 1998), enzyme activity remains in the pellet following permeabilization and centrifugation of sperm samples (Welch et al., 2000). Control values for GAPDHS activity are determined from untreated sperm samples incubated under identical conditions as the treated samples.

Vital dye staining with propidium iodide and SYBR 14 (LIVE/DEAD Sperm Viability Kit from Molecular Probes Inc., Eugene, Oreg., United States of America) is used to test sperm viability following treatment with cell-permeable GAPDHS modulators (Hagaman et al., 1998).

Sperm treated with cell-permeable GAPDHS modulators are also tested for their ability to successfully participate in in vitro fertilization according to Cho et al., 1998.

Sperm motility is tested using computer-aided sperm analysis (CASA) as described in Hagaman et al., 1998 and Slott et al., 1993, including additional analyses to assess hyperactivated motility as described by Cancel et al., 2000. Using this procedure, the following measures of sperm motility are quantified: curvilinear velocity (VCL, time-average velocity of a sperm head along its actual curvilinear trajectory); average path velocity (VAP, time-average velocity of a sperm head along its spatial average trajectory); straight line velocity (VSL, time-average velocity of a sperm head along the straight line between its first and last detected positions); amplitude of head displacement (ALH, magnitude of lateral displacement of a sperm head about its spatial trajectory); beat cross frequency (BCF, time-average rate at which the VCL trajectory crosses the VAP trajectory); linearity (linearity of the curvilinear trajectory=VSL/VCL×100); wobble (a measure of the oscillation of the actual trajectory about its spatial average path=VAP/VCL×100); and straightness (linearity of the spatial average path=VSL/VAP×100). The values observed are compared to values observed using control (i.e. untreated) sperm. et al., 1993; Cancel et al., 2000.

Additionally, sperm treated with cell-permeable GAPDHS modulators are tested to determine accumulation of glyceraldehyde 3-phosphate substrate (Racker, 1984), ATP production using a luciferase bioluminescence assay (ATP Bioluminescence Assay kit CLS II, Roche Applied Science, Indianapolis, Ind., United States of America) and oxygen consumption using an oxygen probe calibrated to air-saturated medium to assess mitochondrial function. Sperm functional assays include untreated sperm incubated under identical conditions as controls.

### Example 16

### Testing of Sperm Function following Administration of GAPDHS Modulators to Animals

Modulators are tested for the ability to modulate a biological activity of GAPDHS using the techniques disclosed in Bone et al., 2000.

Briefly, rats or mice are treated with a modulator, for example by feeding the modulator to different rats in varying amounts, over a time course of 0 to 14 days. After treatment, spermatozoa are isolated and the enzyme activities of glycolytic enzymes present in the spermatozoa including GAPDHS and triose phosphate isomerase (TPI) are tested in sperm sonicates. The enzyme activity of a control enzyme (e.g. hexokinase) is also tested.

Additionally, NMR spectroscopy is used to estimate glycolytic turnover by testing the acidification of the exogenous medium.

Vital dye staining with propidium iodide and SYBR 14 (LIVE/DEAD Sperm Viability Kit, available from Molecular Probes Inc., Eugene, Oreg., United States of America) is used to test sperm viability according to Hagaman et al., 1998.

Sperm from treated animals is also tested for its ability to successfully participate in in vitro fertilization according to Cho et al., 1998.

Sperm motility is tested using computer-aided sperm analysis (CASA) as described in Hagaman et al., 1998 and Slott et al., 1993, including additional analyses to assess hyperactivated motility as described by Cancel et al., 2000. Using this procedure, the following measures of sperm motility are quantified: curvilinear velocity (VCL, time-average velocity of a sperm head along its actual curvilinear trajectory); average path velocity (VAP, time-average velocity of a sperm head along its spatial average trajectory); straight line velocity (VSL, time-average velocity of a sperm head along the straight line between its first and last detected positions); amplitude of head displacement (ALH, magnitude of lateral displacement of a sperm head about its spatial trajectory); beat cross frequency (BCF, time-average rate at which the VCL trajectory crosses the VAP trajectory); linearity (linearity of the curvilinear trajectory=VSL/VCL×100); wobble (a measure of the oscillation of the actual trajectory about its spatial average path=VAP/VCL×100); and straightness (linearity of the spatial average path=VSL/VAP×100). The values observed are compared to values observed using control (i.e. untreated) sperm.

### Example 17

### In Vivo Testing of GAPDHS Modulators

Modulators are further tested with regard to induction of temporary male infertility in vivo. Male mice and/or rats are treated with varying doses of modulators for various periods of time, and then mated to numerous fertile, ovulating females. Treatment continues while the matings are ongoing. Vaginal plugs are checked each day to ensure that the animals are copulating, and females that have been inseminated are replaced with new females. The ability of treated animals to sire progeny is compared to that of untreated control animals to determine the efficacy of treatment and the duration of treatment required to produce infertility.

After each male has inseminated about 10 females, treatment with the modulator is discontinued and the males continue to be provided with breeding partners. This is continued until the temporary nature (including efficiency and duration) of the male infertility is established.

To determine if the modulators can be administered to females to prevent fertilization, similar mating studies are conducted with treated females (various doses and time periods) and fertile untreated males.

For compounds that produce male infertility, further analyses of sperm function are assessed as described hereinabove and in Bone et al., 2000. Briefly, mice or rats are treated with a modulator, for example by feeding the modulator to different rats in varying amounts, over a time course of 0 to 14 days. After treatment, spermatozoa are isolated and Gapdhs enzyme activity and parameters of sperm function are assessed. This approach provides a method of assessing the efficacy and specificity of Gapdhs modulators administered in vivo. In addition, it allows testing of compounds that require metabolism to produce an active modulator of Gapdhs.

In order to test the ability of the modulators disclosed herein to inhibit human GAPDHS in a physiological context, homozygous Gapdhs knockout mice (i.e. mice lacking Gapdhs gene function) are produced by standard techniques. Homozygous mice are infertile, as confirmed by breeding genotyped homozygous knockout mice to wild type females (Examples 9-15).

Concurrently, a transgenic mouse line is produced that correctly expresses a full-length wild type human GAPDHS polypeptide. Transgenic mice have been used to delimit the upstream regulatory region required for GAPDHS expression (Welch et al., 1994). A series of constructs (A, B, C, D) with progressive 5′ deletions of the GAPDHS promoter ligated to the E. coli lacZ (β-galactosidase) reporter gene were introduced into the mouse genome by pronuclear injection. The 3′ untranslated region of Gapdhs (127 bp) was ligated to the C-terminus of the reporter gene in all constructs. Like native GAPDHS, β-galactosidase expression occurred in condensing spermatids during the late stages of spermatogenesis. Specific expression of β-galactosidase was detected in transgenic mice containing constructs with 1350 bp (A), 626 bp (B) or 336 bp (C) of Gapdhs promoter sequence 5′ to the transcription initiation codon. However, reporter gene expression was variable in the testis of transgenic mice containing 162 bp (construct D) of Gapdhs promoter. The promoter and 3′-untranslated region of construct C are used in constructing a transgenic line with restricted expression of human GAPDHS during the late stages of spermatogenesis. This transgenic mouse line is bred to females from the Gapdhs knockout line to produce a hybrid line that expresses human GAPDHS and is homozygous for the Gapdhs knockout. Thus, the appropriate mice should express human GAPDHS in place of Gapdhs. Males from these mice (referred to herein as Gapdhs-minus, GAPDHS-plus) are then treated with a modulator, and tested for fertility, GAPDHS enzyme activity, and parameters of sperm function (motility, etc). Potential side effects of the modulator are also tested by careful phenotypic analysis of the treated animals. Standard procedures for phenotypic analysis are used (see the website of the Jackson Laboratory).

As an alternative to creating a transgenic mouse line that expresses a full-length wild type GAPDHS protein, a second transgenic mouse line is created that expresses a chimeric protein comprising the proline rich sequence from Gapdhs (e.g. amino acids 1-103 SEQ ID NO: 2) fused in-frame to amino acids 74-408 of GAPDHS (SEQ ID NO: 6). This chimeric protein is expected to interact appropriately with the mouse sperm fibrous sheath by virtue of its mouse N-terminal proline-rich sequence, but would have the ligand-binding and catalytic domains from human GAPDHS (amino acids 74-408 of SEQ ID NO: 6).

This transgenic mouse line is then used to create the Gapdhs-minus, GAPDHS-plus mouse line as described hereinabove.

### REFERENCES

All publications mentioned herein are incorporated herein by reference for the purpose of describing and disclosing, for example, the cell lines, constructs, and methodologies that are described in the publications, which might be used in connection with the presently described subject matter. The publications discussed above and throughout the text are provided solely for their disclosure prior to the filing date of the present application. Nothing herein is to be construed as an admission that the inventors are not entitled to antedate such disclosure by virtue of prior invention.
Agrawal S (ed.) (1993) Methods in Molecular Biology, volume 20, Humana Press, Totowa, N.J., United States of America.
Altschul S F et al. (1990) J Mol Biol 215:403-410.
Anderson M J & Dixon A F (2002) Nature 416:496.
Aronov A M et al. (1999) Proc Natl Acad Sci USA 96:4273-4278.
Aronov A M et al. (1998) J Med Chem 41:4790-4799.
Ausubel F M et al., (eds.) (1989) Current Protocols in Molecular Biology. Wiley, New York, United States of America.
Bartlett et al. (1989) Special Publ, Royal Chem Soc 78: 182-96.
Blom N et al. (1999) J Mol Biol 294:1351-1362.
Bohm H J (1992) J Comput Aid Mol Des 6:593-606.
Bone W et al. (2000) J Reproduction Fertility 118:127-135.
Bressi J C et al. (2000) J Med Chem 43:4135-4150.
Bressi J C et al. (2001) J Med Chem 44:2080-2093.
Brooks B R et al. (1983) J Comp Chem 4:187-217.
Bunch D O et al. (1998) Biol Reprod 58:83441.
Cancel A M et al. (2000) Hum Reprod 15:1322-8.
Case D A et al. (1997) AMBER 5, University of California, San Francisco, San Francisco, Calif., United States of America.
Cho C et al. (1998) Science 281:1857-9.
Cohen N C et al. (1990) J Med Chem 33:883-894.
Connolly M L (1983) J Appl Cryst 16:548-558.
Cooper T G (1984) Gamete Res 9:55-74.
Dix D J et al. (1996) Proc Natl Acad Sci USA 93:3264-3268.
Duée E et al. (1996) J Mol Biol 257:814-838.
Ebel S et al. (1992) Biochem 31:12083-6.
Eisen M B et al. (1994) Proteins 19:199-221.
Fraser L R & Quinn P J (1981) J Reprod Fertil 61:25-35.
Garcia C K et al. (1995) J Biol Chem 270:1843-1849.
Goeddel (1990) Gene Expression Technology, Methods in Enzymology, volume 185, Academic Press, San Diego, Calif., United States of America.
Goodford P J (1985) J Med Chem 28:849-857.
Goodsell D S & Olson A J (1990) Proteins 8:195-202.
Hagaman J R et al. (1998) Proc Natl Acad Sci USA 95:2552-7.
Hagler A T et al. (1979) J Amer Chem Soc 101:5122-5130.
Halestrap A P & Price N T (1999) Biochem J 343:281-299.
Henikoff S & Henikoff J G (1992) Proc Natl Acad Sci USA 89:10915-10919.
Hoppe P C (1976) Biol Reprod 15:39-45.
Hoshi K et al. (1991) Tohoku J Exp Med 165:99-104.
Jones A R (1978) Life Sci 23:1625-1645.
Jones A R (1998) J Reprod Fertil Suppl 53:227-34.
Jones A R & Cooper T G (1999) Intl J Androl 22:130-138.
Karlin S & Altschul S F (1993) Proc Natl Acad Sci USA 90:5873-5877.
Kuntz I D et al. (1982) J Mol Biol 161:269-288.
Lambert (1997) in Practical Application of Computer-Aided Drug Design, (Charifson, ed.) pp. 243-303, Marcel-Dekker, New York, N.Y., United States of America.
Lattman E (1985) in Methods in Enzymology, volume 115, Academic Press, San Diego, Calif., United States of America, pages 55-77.
Lee B & Richards F M (1971) J Mol Biol 55:379-400.
Luty B A et al. (1995) J Comp Chem 16:454-464.
Mahadevan M M et al. (1997) Hum Reprod 12:119-123.
Martin Y C (1992) J Med Chem 35:2145-2154.
McRee (1993) Practical Protein Crystallography, Academic Press, New York, N.Y., United States of America.
Miki K et al. (2004) Proc Natl Acad Sci USA 101:16501-16506.
Miki K et al. (2002) Dev Biol 248:331-42.
Miranker A & Karplus M (1991) Proteins 11:29-34.
Mori C et al. (1992) Biol Reprod 46:859-868.
Murthy M R et al. (1980) J Mol Biol 138:859-872.
Nagradova N K (2001) Biochem (Mosc) 66:1067-76.
Narisawa S et al. (2002) Mol Cell. Biol. 22:5554-5562.
Navaza J & Saludjian P (1997) Meth Enzymol. 276A:581-94.
Navia M A & Murcko M A (1992) Curr Opin Struct Biol 2:202-210.
Needleman S B & Wunsch C D (1970) J Mol Biol 48:443-453.
Nishibata Y & Itai A (1991) Tetrahedron 47: 8985-8990.
Oberländer G et al. (1994) J Reprod Fertility 100 551-559.
PCT International Publication Number WO 93/25521
Pearlman D A et al. (1995) Comput Phys Commun 91:1-41.
Pearson W R & Lipman D J (1988) Proc Natl Acad Sci USA 85:2444-2448.
Racker E (1983) Methods of Enzymatic Analysis. 3^{rd} edition (Bergmeyer H U, ed.), Weinheim, Deerfield Beach, Fla., United States of America 6:561-5.
Rarey M et al. (1996) J Comput Aid Mol Des 10:41-54.
Rossmann M G (ed.) (1972) The Molecular Replacement Method, Gordon & Breach, New York, N.Y., United States of America.
Sambrook J & Russell D W (2001) Molecular Cloning: A Laboratory Manual, Third Edition, Cold Spring Harbor Laboratory, Cold Spring Harbor, N.Y., United States of America.
Schmalhausen E V et al. (1997) FEBS Lett 414:247-52.
Schulz G E & Schirmer R H (1979) Principles of Protein Structure, Springer-Verlag, New York, N.Y., United States of America.
Shen Y Q et al. (2000) J Struct Biol 130:1-9.
Slott V L et al. (1993) Fundam Appl Toxicol 21:298-307.
Smith T F & Waterman M (1981) Adv Appl Math 2:482-489.
Stevenson D & Jones A R (1985) J Reprod Fertil 74:157-165.
Suarez S S (1996) J Androl 17:331-335.
Tibanyenda N et al., (1984) Eur J Biochem 139:19.
Tijssen P (1993) Laboratory Techniques in Biochemistry and Molecular Biology, Elsevier, New York, N.Y., United States of America.
U.S. Pat. No. 5,234,933
U.S. Pat. No. 5,326,902
U.S. Pat. No. 5,352,660
U.S. Pat. No. 5,422,245
U.S. Pat. No. 5,645,999
U.S. Pat. No. 5,739,278
U.S. Pat. No. 5,786,152
U.S. Pat. No. 5,837,479
U.S. Pat. No. 6,008,033
Velick S (1955) in Methods in Enzymology, volume 1, Academic Press, New York, N.Y., United States of America.
Verlinde C L M J et al. (1994) J Med Chem 37:3605-3613.
Vriend G (1990) J Mol Graph 8:52-56.
Welch J E et al. (1995) Dev Genetics 16:179-189.
Welch J E et al. (2000) J Androl 21:328-338.
Welch J E et al., (1992) Biol Reprod 46:869-878.
Yanagimachi R (1994) Mammalian fertilization. in The Physiology of Reproduction (Knobil E & Neill J eds), Raven Press, New York, N.Y., United States of America.

It will be understood that various details of the presently disclosed subject matter can be changed without departing from the scope of the presently disclosed subject matter. Furthermore, the foregoing description is for the purpose of illustration only, and not for the purpose of limitation.

1. A method of reducing motility of sperm produced in a subject, the method comprising administering an effective amount of a male germ cell-specific isoform of glyceraldehyde 3-phosphate dehydrogenase (GAPDHS) activity inhibitor to the subject.

2. The method of claim 2, wherein the subject is a mammal selected from the group consisting of a human, a mouse, and a rat.

3. The method of claim 2, wherein:
(i) the mammal is a human and the inhibitor interacts with one or more of the following residues in a human male germ cell-specific isoform of glyceraldehyde 3-phosphate dehydrogenase (GAPDHS) of SEQ ID NO: 6: N81, G82, F83, G84, R85, I86, G87, L89, N105, D106, P107, F108, C150, K151, E152, P153, E168, S169, T170, V172, Y173, L174, S175, A178, S193 P197, S223, C224, T225, H251, S252, Y253, T254, A255, T256, Q257, K258, S264, R265, A267, R269, D270, G271, I279, P280, A281, S282, T283, G284, A304, R306, P310, N388, E389, Y392, and S393;
(ii) the mammal is a mouse and the inhibitor interacts with one or more of the following residues in a mouse male germ cell-specific isoform of glyceraldehyde 3-phosphate dehydrogenase (Gapdhs) of SEQ ID NO: 2: N111, G112, F113, G114, R115, I116, L119, N135, D136, P137, F138, C180, K181, D182, P183, E198, C199, T200, V202, Y203, L204, S205, A208, T223, P227, S253, C254, T255, H281, S282, Y283, T284, A285, T286, Q287, K288, S294, K295, D297, R299, G300, G301, I309, P310, S311, S312, T313, G314, A334, R336, P340, N418, E419, Y422, and S423; or
(iii) the mammal is a rat and the inhibitor interacts with one or more of the following residues in a rat male germ cell-specific isoform of glyceraldehyde 3-phosphate dehydrogenase (Gapdhs) of SEQ ID NO: 4: N105, G106, F107, G108, R109, I110, L113, N129, D130, P131, F132, C174, K175, E176, P177, E192, A193, T194, V196, Y197, L198, S199, A202, T217, P221, S247, C248, T249, H275, A276, Y277, T278, A279, T280, Q281, K282, S288, K289, D291, R293, G294, G295, I303, P304, S305, S306, T307, G308, A328, R330, P334, N412, E413, Y416, and S417.

4. The method of claim 3, wherein the interaction is selected from the group consisting of a van der Waals interaction, a hydrophobic interaction, hydrogen bonding, and combinations thereof.

5. The method of claim 1, wherein the inhibitor is selected from the group consisting of the molecules presented in Tables 7, 9, and 11.

6. The method of claim 5, wherein the inhibitor is selected from the group consisting of LT_{—}00249157, 11K_{—}064, T0501_{—}7749, T0511_{—}4909, T0501_{—}7933, T0506_{—}9350, and T0515_{—}4928.

7. A method of screening a candidate composition for an effect on sperm motility, the method comprising:
(a) contacting a male germ cell-specific isoform of glyceraldehyde 3-phosphate dehydrogenase (GAPDHS) with a candidate compound;
(b) determining an effect of the candidate compound on a biological activity of the GAPDHS; and
(c) determining whether the candidate compound has an effect on sperm motility based on the effect of the candidate compound on a biological activity of the GAPDHS.

8. The method of claim 7, wherein the candidate compound is screened for selective inhibition of a male germ cell-specific isoform of glyceraldehyde 3-phosphate dehydrogenase (GAPDHS) as compared to a somatic glyceraldehyde 3-phosphate dehydrogenase (GAPDH) enzyme.

9. The method of claim 7, wherein the male germ cell-specific isoform of glyceraldehyde 3-phosphate dehydrogenase (GAPDHS) is a recombinant GAPDHS.

10. The method of claim 7, wherein the contacting is carried out in vitro.

11. The method of claim 7, wherein the contacting is carried out by administering the candidate compound to a test subject.

12. The method of claim 7, wherein the effect is an inhibitory effect.

13. The method of claim 7, wherein the male germ cell-specific isoform of glyceraldehyde 3-phosphate dehydrogenase (GAPDHS) is selected from the group consisting of a human GAPDHS, a mouse Gapdhs, and a rat Gapdhs.

14. The method of claim 13, wherein the candidate compound is designed to interact with one or more amino acids selected from the group consisting of:
(1) N81, G82, F83, G84, R85, I86, G87, L89, N105, D106, P107, F108, C150, K151, E152, P153, E168, S169, T170, V172, Y173, L174, S175, A178, S193 P197, S223, C224, T225, H251, S252, Y253, T254, A255, T256, Q257, K258, S264, R265, A267, R269, D270, G271, I279, P280, A281, S282, T283, G284, A304, R306, P310, N388, E389, Y392, and S393 of SEQ ID NO: 6;
(2) N111, G112, F113, G114, R115, I116, L119, N135, D136, P137, F138, C180, K181, D182, P183, E198, C199, T200, V202, Y203, L204, S205, A208, T223, P227, S253, C254, T255, H281, S282, Y283, T284, A285, T286, Q287, K288, S294, K295, D297, R299, G300, G301, I309, P310, S311, S312, T313, G314, A334, R336, P340, N418, E419, Y422, and S423 of SEQ ID NO: 2; and
(3) N105, G106, F107, G108, R109, I110, L113, N129, D130, P131, F132, C174, K175, E176, P177, E192, A193, T194, V196, Y197, L198, S199, A202, T217, P221, S247, C248, T249, H275, A276, Y277, T278, A279, T280, Q281, K282, S288, K289, D291, R293, G294, G295, I303, P304, S305, S306, T307, G308, A328, R330, P334, N412, E413, Y416, and S417 of SEQ ID NO: 4.

15. The method of claim 14, wherein the interaction is selected from the group consisting of a van der Waals interaction, a hydrophobic interaction, hydrogen bonding, and combinations thereof.

16. A method of modeling an interaction between a male germ cell-specific isoform of glyceraldehyde 3-phosphate dehydrogenase (GAPDHS) and a ligand, the method comprising:
(a) providing a homology model of a target GAPDHS;
(b) providing atomic coordinates of a ligand; and
(c) docking the ligand with the homology model to form a GAPDHS/ligand model.

17. The method of claim 16, further comprising screening for selective inhibition of a male germ cell-specific isoform of glyceraldehyde 3-phosphate dehydrogenase (GAPDHS) as compared to a somatic glyceraldehyde 3-phosphate dehydrogenase (GAPDH) enzyme.

18. The method of claim 16, wherein the male germ cell-specific isoform of glyceraldehyde 3-phosphate dehydrogenase (GAPDHS) is human GAPDHS, a mouse Gapdhs, or a rat Gapdhs, and further wherein the ligand is designed to interact with one or more amino acids selected from the group consisting of:
(1) N81, G82, F83, G84, R85, I86, G87, L89, N105, D106, P107, F108, C150, K151, E152, P153, E168, S169, T170, V172, Y173, L174, S175, A178, S193 P197, S223, C224, T225, H251, S252, Y253, T254, A255, T256, Q257, K258, S264, R265, A267, R269, D270, G271, I279, P280, A281, S282, T283, G284, A304, R306, P310, N388, E389, Y392, and S393 of SEQ ID NO: 6;
(2) N111, G112, F113, G114, R115, I116, L119, N135, D136, P137, F138, C180, K181, D182, P183, E198, C199, T200, V202, Y203, L204, S205, A208, T223, P227, S253, C254, T255, H281, S282, Y283, T284, A285, T286, Q287, K288, S294, K295, D297, R299, G300, G301, I309, P310, S311, S312, T313, G314, A334, R336, P340, N418, E419, Y422, and S423 of SEQ ID NO: 2; and
(3) N105, G106, F107, G108, R109, I110, L113, N129, D130, P131, F132, C174, K175, E176, P177, E192, A193, T194, V196, Y197, L198, S199, A202, T217, P221, S247, C248, T249, H275, A276, Y277, T278, A279, T280, Q281, K282, S288, K289, D291, R293, G294, G295, I303, P304, S305, S306, T307, G308, A328, R330, P334, N412, E413, Y416, and S417 of SEQ ID NO: 4.

19. The method of claim 18, wherein the interaction is selected from the group consisting of a van der Waals interaction, a hydrophobic interaction, hydrogen bonding, and combinations thereof.

20. A method of designing a modulator of a male germ cell-specific isoform of glyceraldehyde 3-phosphate dehydrogenase (GAPDHS), the method comprising:
(a) selecting a candidate GAPDHS ligand;
(b) determining which amino acid or amino acids of the GAPDHS interact with the ligand using a three-dimensional model of a GAPDHS;
(c) identifying in a biological assay for GAPDHS activity a degree to which the ligand modulates the activity of the GAPDHS;
(d) selecting a chemical modification of the ligand wherein the interaction between the amino acids of the GAPDHS and the ligand is predicted to be modulated by the chemical modification;
(e) synthesizing a ligand having the chemical modified to form a modified ligand;
(f) identifying in a biological assay for GAPDHS activity a degree to which the modified ligand modulates the biological activity of the GAPDHS; and
(g) comparing the biological activity of the GAPDHS in the presence of modified ligand with the biological activity of the GAPDHS in the presence of the unmodified ligand, whereby a modulator of a GAPDHS is designed.

21. The method of claim 20, further comprising screening for selective inhibition of male germ cell-specific isoform of glyceraldehyde 3-phosphate dehydrogenase (GAPDHS) as compared to a somatic glyceraldehyde 3-phosphate dehydrogenase (GAPDH) enzyme.

22. The method of claim 20, wherein the male germ cell-specific isoform of glyceraldehyde 3-phosphate dehydrogenase (GAPDHS) is a human GAPDHS a mouse Gapdhs, or a rat Gapdhs.

23. The method of claim 20, wherein the modified ligand is designed to interact with one or more amino acids selected from the group consisting of:
(1) N81, G82, F83, G84, R85, I86, G87, L89, N105, D106, P107, F108, C150, K151, E152, P153, E168, S169, T170, V172, Y173, L174, S175, A178, S193 P197, S223, C224, T225, H251, S252, Y253, T254, A255, T256, Q257, K258, S264, R265, A267, R269, D270, G271, I279, P280, A281, S282, T283, G284, A304, R306, P310, N388, E389, Y392, and S393 of SEQ ID NO: 6;
(2) N111, G112, F113, G114, R115, I116, L119, N135, D136, P137, F138, C180, K181, D182, P183, E198, C199, T200, V202, Y203, L204, S205, A208, T223, P227, S253, C254, T255, H281, S282, Y283, T284, A285, T286, Q287, K288, S294, K295, D297, R299, G300, G301, I309, P310, S311, S312, T313, G314, A334, R336, P340, N418, E419, Y422, and S423 of SEQ ID NO: 2; and
(3) N105, G106, F107, G108, R109, I110, L113, N129, D130, P131, F132, C174, K175, E176, P177, E192, A193, T194, V196, Y197, L198, S199, A202, T217, P221, S247, C248, T249, H275, A276, Y277, T278, A279, T280, Q281, K282, S288, K289, D291, R293, G294, G295, I303, P304, S305, S306, T307, G308, A328, R330, P334, N412, E413, Y416, and S417 of SEQ ID NO: 4.

24. The method of claim 23, wherein the interaction is selected from the group consisting of a van der Waals interaction, a hydrophobic interaction, hydrogen bonding, and combinations thereof.

25. The method of claim 20, wherein the method further comprises repeating steps (a) through (f) if the biological activity of the male germ cell-specific isoform of glyceraldehyde 3-phosphate dehydrogenase (GAPDHS) in the presence of the modified ligand varies from the biological activity of the GAPDHS in the presence of the unmodified ligand.
